(19) United States
(12) Patent Application Publication
(10) Pub. No.: US 2003/0138864 A1
(43) Pub. Date: Jul. 24, 2003

(54) Method for identifying an enzyme to design anti-cancer compounds

(76) Inventors: Hideo Ishitsuka, Tokyo (JP); Hisafumi Okabe, Yokohama-Shi (JP); Nobuo Shimma, Chigasaki-shi (JP); Takuo Tsukuda, Odawara-shi (JP); Isao Umeda, Yokohama-shi (JP)

Correspondence Address: HOFFMANN-LA ROCHE INC., PATENT LAW DEPARTMENT, 340 KINGSLAND STREET, NUTLEY, NJ, 07110

(21) Appl. No.: 10/301,460

(22) Filed: Nov. 21, 2002

(30) Foreign Application Priority Data
Nov. 23, 2001 (EP) 01127401.6
Dec. 19, 2001 (EP) 01130245.2
Mar. 12, 2002 (EP) 02005298.1

(51) Int. Cl. G01N 33/574; A61K 31/7072; A61K 31/513; A61K 31/4745; A61K 31/337; C07H 19/048; C07D 498/14; C 07D 4 5/02; C 07D 3 5/14

(52) U.S. Cl. 435/7.23; 549/510; 536/28.1; 546/47; 544/314; 514/49; 514/283; 514/449; 514/269

(57) ABSTRACT

The present invention relates to a method for identification of enzymes that are preferentially expressed in certain tumor tissue as compared with rapidly growing normal cells or tissue, use of said enzymes for the compound design to generate an active anti-cancer substance selectively in tumor tissue, compounds designed based on said enzymes, their pharmaceutically acceptable salts as well as pharmaceutical composition thereof.

### FIELD OF THE INVENTION

The present invention relates to a method for identifying enzymes that are preferentially expressed in certain tumor tissue as compared with rapidly growing normal cells or tissue, use of said enzymes for the compound designed to generate an active anti-cancer substance selectively in tumor tissue, compounds designed based on said enzymes, their pharmaceutically acceptable salts as well as pharmaceutical composition thereof.

### BACKGROUND OF THE INVENTION

One of the most serious but most important issues in the use of medicines is side effects. Side effects of drugs are mainly caused by non-specific action of drugs; drugs interact with and affect not only target molecules of the drugs but also other molecules that play important roles in maintaining normal physiological processes. Another major cause of the side effects results from non-specific distribution of drugs in many tissues; drugs are incorporated into not only tissues that are to be affected but also to other tissues that should remain unaffected to keep normal physiological functions. Target molecules of most anti-cancer drugs are widely expressed in many tissues and not specific to certain tissues. On the other hand, disease is usually caused by disregulation of certain molecules in certain tissues. Thus, to avoid side effects, it is necessary to establish methods by which the drugs affect certain molecules only in certain tissues that cause diseases, and present drugs designed by such methods.

Among many diseases, side effects of drugs are particularly concerned in the treatment of cancer patients. Cytotoxic drugs have been widely used for the treatment of cancer and will continue to be regularly used for cancer chemotherapy at least in the next decade. However, the use of cytotoxic drugs is limited due to their insufficient efficacy and severe side effects. In tumor tissue, many cytotoxic drugs including 5-FU, 2′-deoxycytidines, methotrexate, camptothecins and taxanes affect tumor cells at S or M phase of cell cycle, the time when DNA synthesis or mitosis occurs. However, growing tumor cells in tumor tissues are at various stages of cell cycles, and only a small portion of tumor cells is at S or M phase. Therefore, ideal drug exposure time should be, at least, longer than that required for the completion of one cell cycle (ranging from 20 to 40 hours), and ideal dosing regimen for cytotoxic drugs is consecutive daily or continuous treatment to affect all the cancer cells present in tumor tissue. However, cytotoxic drug treatment in such dosing regimens cause severe toxicity on rapidly growing normal cells, particularly on hematopoietic progenitor cells and intestinal crypt cells. Myelosuppression, a condition that is caused by the toxicity on hematopoietic progenitor cells, is the most frequent among various types of side effects of cytotoxic drugs and often results in impairment of host immune responses and fetal infections. Once myelosuppression occurs, it generally takes 2 to 3 weeks to recover from the myelotoxicity, and this is the main reason why many cytotoxic drugs are given once every 3 to 4 weeks. However, this intermittent dosing regimen results in insufficient efficacy of most existing cytotoxic drugs.

Several novel anti-tumor agents with new modes of actions are currently under development. However, they also have some safety problems due to their insufficient tumor selectivity. Indeed, major toxicities of farnesyltransferase inhibitors and epidermal growth factor (EGF) receptor tyrosine kinase inhibitors appear to be myelotoxicity and skin rush, respectively. This is presumably because the target enzyme or protein are over-expressed not only in tumor tissues, but also other normal tissue such as bone marrow and skin.

On the other hand, capecitabine (an oral fluoropyrimidine) is a cytotoxic drug that is sequentially converted to the active drug 5-FU by enzymes that are highly expressed in the liver and tumors, but not in the growing bone marrow cells [Miwa. M. et al. Design of oral fluoropyrimidine carbamate, capecitabine, which generates 5-furuolouracil selectively in tumors by enzymes concentrated in human liver and cancer tissue. Eur. J. Cancer 34, 1274-1281 (1998)]. As the result, it gives high concentrations of 5-FU selectively in tumor tissue and shows better efficacy profiles compared with those of 5-FU. In addition, it causes little myelotoxicity. These characteristics make the drug available for daily treatment at high dosages even for long duration. It is now being prescribed for the treatment of breast, colorectal and other cancers. Nevertheless, it is still difficult to identify anti-cancer drugs having higher efficacy and safer margins like capecitabine, because there is no established way to pinpoint enzymes and/or proteins among the large number of those that are expressed in various tissues.

### SUMMARY OF THE INVENTION

The present invention relates to methods of identifying enzymes for designing compounds that can be converted to active substances selectively in tumors but not in normal growing cells (hereafter called Tumor-Targeting Cytotoxics (TTC)), particularly granulocyte progenitors that are predominantly present in bone marrow. Tumor-targeting cytotoxics , would have tumor selective action with little myelotoxicity. Such compounds can be safely given at higher doses for long periods showing more improved safety and efficacy profiles as compared with those of existing cytotoxics. These compounds therefore could reduce hospitalization that relates to the side effects and can be safely prescribed to outpatients. Other advantages of tumor-targeting cytotoxics include that they will enable us to pursue individualized healthcare therapy (tailored therapy) by measuring the expression levels of their activation enzymes (TTC-activation enzymes). Individual tumors expressing high levels of TTC-activation enzymes will efficiently generate active drugs from tumor-targeting cytotoxics, and therefore, are likely to be highly susceptible to the tumor-targeting cytotoxics.

One aspect of the present invention provides a method for identifying enzymes to design anti-cancer compounds in which the enzyme is converted to active substances selectively in tumors, which comprises measuring the expression levels of genes or proteins in human tissue or cells from normal and tumor origin, comparing the measured expression levels and selecting the enzymes of which mRNA and protein levels in tumor tissue are higher by more than two-fold than in normal growing-hematopoietic progenitors, intestine, or skin.

Another aspect of the present invention provides methods of identifying anti-cancer compounds that can be converted to active substances selectively in tumors comprising the steps of generating cells expressing an enzyme of which protein levels in tumor tissue are higher by more than two-fold as compared to normal cells or tissue and determining growth inhibitory activities of said anti-cancer compounds.

Another aspect of this invention provides anti-cancer compounds of the formula (I),

X—Y-Q (I)

wherein

X is a pro-moiety that is designed to generate an active anti-cancer substance (Q-Y—H) selectively in tumors by the enzymes according to the present invention;

Q-Y— is a radical derived from the active anti-cancer substance (Q-Y—H) in which Y is —O—, —S— or —N—,

and pharmaceutically acceptable salts thereof.

It is another aspect of the invention to provide anti-cancer compounds represented by the formula (II),

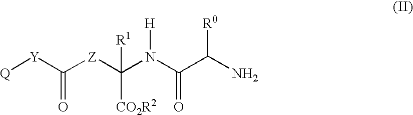

wherein

Q and Y are the same as defined above,

R⁰ is a side chain of natural or non-natural amino acid,

Z is (C1-C3)alkylene or —O—CH(R³)—, wherein R³ is hydrogen or straight (C1-C4)alkyl,

R¹ is hydrogen or methyl, and

R² is hydrogen, branched (C3-C10)alkyl or (C3-C8)cycloalkyl,

and pharmaceutically acceptable salts thereof.

It is another aspect of the invention to provide anti-cancer compounds represented by the formula (III),

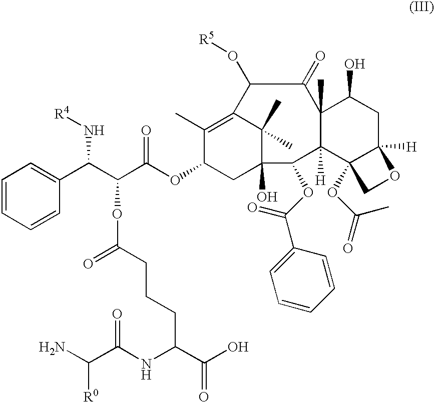

wherein

R⁰ is the same as defined above,

R⁴ is benzoyl or tert-butoxycarbonyl, and

R⁵ is hydrogen or acetyl,

and pharmaceutically acceptable salts thereof.

It is another aspect of the invention to provide anti-cancer compounds represented by the formula (IV),

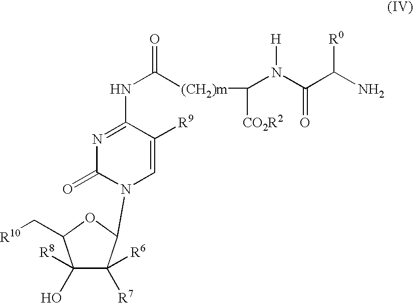

wherein

R⁰, R¹, R² and R³ are the same as defined above,

R⁶ is hydrogen, fluorine, hydroxyl or cyano,

R⁷ is hydrogen, fluorine or hydroxy,

or R⁶ and R⁷ taken together to form methylidene or fluoromethylidene,

R⁸ is hydrogen or ethynyl,

R⁹ is hydrogen, fluorine, vinyl or ethynyl, and

R¹⁰ is hydrogen or hydroxy

and pharmaceutically acceptable salts thereof.

It is another aspect of the invention to provide anti-cancer compounds represented by the formula (V),

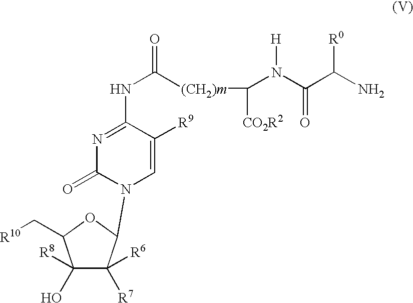

wherein

m is an integer of 2 or 3,

R⁰, R², R⁶, R⁷, R⁸, R⁹ and R¹⁰ are the same as defined above,

and pharmaceutically acceptable salts thereof

It is another aspect of the invention to provide anti-cancer compounds represented by the formula (VI),

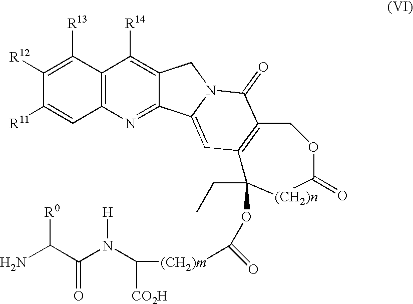

wherein

m is an integer of 1 to 3,

n is an integer of 0 to 1,

R⁰ is the same as defined above,

R¹¹ is hydrogen or fluorine,

R¹² is hydrogen, fluorine, methyl or hydroxy,

R¹³ is hydrogen, amino, nitro, or (dimethylamino)methyl,

R¹⁴ is hydrogen, (C1-C4)alkyl, 4-methylpiperazinylmethyl, tert-butoxyiminomethyl or R¹³ and R¹⁴, or R¹¹ and R¹² taken together may form five or six membered ring which may contain one or two hetero atom(s), and may be optionally substituted with (C1-C8)alkyl, amino, (C1-C8)alkylamino, and di-(C1-C4)alkylamino,

and pharmaceutically acceptable salts thereof.

In the present invention the term “(C1-C3)alkylene” refers to a biradical branched or unbranched hydrocarbon chain containing 1 to 3 carbon atom(s), such as methylene, ethylene, propylene and trimethylene, most preferably ethylene.

In the present invention the term “—O—CH(R³)—” refers to —O—CH₂—, —O—CH(CH₃)—, —O—CH(CH₂CH₃)—, —O—CH(CH₂CH₂CH₃)—, —O—CH(CH₂CH₂CH₂CH₃)—; preferably —O—CH₂—, —O—CH(CH₃)—, and most preferably —O—CH(CH₃)—.

The term “acetyl” refers to CH₃CO—.

The term “cycloalkyl” signifies a saturated, cyclic hydrocarbon group with 3 to 7 carbon atoms, preferably with 4 to 7 carbon atoms, more preferably 4 to 6 carbon atoms, i.e. cyclopropyl, cyclobutyl, cyclopentyl and cyclohexyl and the like.

The term “hetero atom” refers to oxygen, nitrogen and sulfur.

The term “mono- and di-alkylamino” refers to an amino substituted with alkyl or di-alkyl as defined above, i.e. alkyl-NH— and di-alklyl-N—. “(C1-C8)alkylamino” refers to methylamino, ethylamino, propylamino, iso-propylamino, butylamino, tert-butylamino, pentylamino, hexylamino, heptylamino and octylamino; preferably butylamino and pentyl amino.

The term “di-(C1-C4)alkylamino” refers to di-methylamino, di-ethylamino, di-propylamino, di-butylamino; preferably di-methylamino and di-ethylamino.

In the definition of R⁰ of formula (II), the term “a side chain of natural amino acid” preferably means the side chain of natural amino acids such as methyl, isopropyl, 2-methylpropyl, 1-methylpropyl, benzyl, indol-3-ylmethyl, 2-(methylthio)ethyl and 4-amonobutyl, 3-aminopropyl; more preferably the term means the side chain of natural lipophilic amino acids such as methyl, 2-methylpropyl, benzyl and indol-3-ylmethyl.

The term “a side chain of non-natural amino acid” preferably means (C5-C12)alkyl, cycloalkylmethyl, substituted or unsubstituted arylmethyl, (cycloalkylthio)methyl, alkylthio-(CH₂)ᵣ— wherein r is an integer of 1 or 2, and the like.

In the above, the term “(C5-C12)alkyl” means straight or branched alkyl chain containing 5 to 12 carbon atoms; more preferably (C8-C12) straight alkyl chain such as n-octyl, nonyl, decyl, undecyl and dodecyl.

The term “alkylthio-(CH₂)ᵣ—” means alkylthio-methyl or alkylthioethyl having a straight, branched alkyl chain containing 2 to 10 carbon atoms such as ethylthiomethyl, ethylthioethyl, n-propylthiomethyl, n-butylthiomethyl, n-pentylthiomethyl, n-octylthiomethyl, n-nonylthiomethyl, n-decylthiomethyl, tert-butylthiomethyl and the like; more preferably ethylthioethyl, n-propylthiomethyl and n-butylthiomethyl.

The term “substituted or unsubstituted arylmethyl” preferably means 4-phenylbenzyl, napht-2-ylmethyl, [4-(4-hydroxyphenoxy)phenyl]methyl and (4-lower-alkoxyphenyl)methyl, in which the term “lower-alkoxy” means straight or branched alkyl chain containing 1 to 6 carbon atom(s); preferably methoxy, ethoxy, propoxy, butoxyl and isopropoxy. The most preferable embodiments of “substituted or unsubstituted arylmethy” are 4-phenylbenzyl, napht-2-ylmethyl, (4-methoxylphenyl)methyl and [4-(4-hydroxyphenoxy)phenyl]methyl.

In the definition of R² of formula (II), the term “branched (C3-C10)alkyl” means branched alkyl chain containing 3 to 6 carbon atom(s), and preferably means iso-propyl, 2-butyl, 3-pentyl, neopentyl and the like: more preferably iso-propyl and 3-pentyl. The term “(C3-C8)cycloalkyl” means a carbon ring consisting of 3 to 8 carbon atoms such as cyclopropyl, cyclobutyl, cyclopentyl, cyclohexyl and the like; more preferably cyclopentyl and cyclohexyl.

In the definition of R³ of formula (II), the term “straight (C1-C4)alkyl” means straight alkyl chain containing 1 to 4 carbon atom(s), and preferably means methyl, ethyl and n-propyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition these salts may be prepared from the addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polymine resins and the like. Preferred salts are the hydrochlorides. Salt free compounds may be prepared by methods known in the art.

In the present invention “the pro-moiety (X)” is a leaving group that is cleaved off in tumors by the enzyme described above after administration of the compound of formula (I) or (II), e.g. (X) is a group a formula

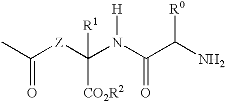

In the present invention, the term “taxans” means taxol [Front. Biotechnol. Pharm. (2000), 1, 336-348], taxotere [J. Med. Aromat. Plant Sci. (2001), 22/4A-23/1A 4-5], IDN 5109 [Chirality, (2000), 12(5/6), 431-441], BMS 188797 [Clinical Cancer Research. 5 (suppl.), 3859, November 1999], BMS184476 [J. Clinical Oncology19:2493-2503, May 1, 2001].

The term “Camptothecins” [(a) Cancer Chemotherapy and Biotherapy: Principle and Practice, 2^{nd} Ed., Lippincott-Ravenmeans, page 463-484, (b) Biochim. Biophys. Acta (1998), 1400(1-3), 107-119] means any compounds having camptothecin skelton such as camptothecin, topotecan, SN-38, 9-aminocamptotecin, 9-nitrocamptothecin, lurtotecan [Br. J. Cancer (1998), 78(10), 1329-1336], DX-8951f [Ann. N.Y. Acad. Sci. (2000), 922(Camptotecins), 260-273], BN-80915 [Anti-cancer Drugs (2001), 12(1), 9-19] and the like.

The term “anti-cancer nucleosides” means a cytidine derivative [Cancer Chemotherapy and Biotherapy: Principle and Practice, 2^{nd} Ed., Lippincott-Ravenmeans, page 213-233] such as DFDC (gemcitabine), DMDC [Clin. Cancer Res. (2000), 6(6), 2288-2294], FMDC [Curr. Opin. Invest. Drugs (PharmaPress Ltd.) (2000), 1(1), 135-140], Ara-C, decitabine [IDrugs (2000), 3(12), 1525-1533], troxacitabine [Clin. Cancer Res. (2000), 6(4), 1574-1588], 2′-cyano-2′-deoxycytidine (CNDAC), 3′-ethynylcytidine (TAS106) [Jpn. J. Cancer Res. (2001), 92(3), 343-351], 5-fluoro-5′-deoxycytidine[Bioorg. Med. Che. Lett., (2000), 8, 1697-1706], 5-viny-5′-deoxycytidine, or an adenosine derivative [Cancer Chemotherapy and Biotherapy: Principle and Practice, 2^{nd} Ed., Lippincott-Ravenmeans, page 235-252] such as fludarabine, cladribine and the like.

The term “dolastatins” means dolastatin 10 [Curr. Pharm. Des. (1999), 5(3), 139-162], dolastatin 14, TZT1027 [Drugs Future (1999), 24(4), 404-409], cemadotin and the like.

The term “anthracyclines” [Cancer Chemotherapy and Biotherapy: Principle and Practice, 2^{nd} Ed., Lippincott-Ravenmeans, page 409-434] means adriamycin, daunomycin, idarubicin and the like.

The term “farnesyl transferase inhibitors” means R115777 [Cancer Res. (2001), 61(1), 131-137], and the like.

The term “EGF receptor tyrosine kinase inhibitors” means ZD1839 [Drugs (2000), 60(Suppl. 1), 33-40], CP 358774 (OSI-774) [J. Pharmacol. Exp. Thr. (1999), 291(2), 739-748], PD 158780 [J. Med. Chem. (2001), 44(3), 429-440], GW2016 and the like.

In the present specification, following symbols or abbreviations refer to following respective compounds.

a) taxol means

[2aR-[2aα,4β,4aβ,6β,9α(αR*,βS*),11α,12α,12aα,12bα]]-β-(benzoylamino)-α-hydroxybenzenepropanoic acid 6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester,

b) taxotere means

[2aR-[2aα, 4β,4aα, 6β,9α(αR*,βS*),11α, 12α, 12aα, 12bα)]-β-[[(1,1-dimethylethoxy)carbonyl]amino]-α-hydroxybenzenepropanoic acid 12b-(acetyloxy)-12-(benzoyloxy)-2a,3,4,4a,5,6,9,10,11,12,12a,12b-dodecahydro-4,6,11-trihydroxy-4a,8,13,13-tetramethyl-5-oxo-7,11-methano-1H-cyclodeca[3,4]benz[1,2-b]oxet-9-yl ester,

c) IDN 5109 means

(2R,3S)-3-[[(1,1-dimethylethoxy)carbonyl]amino]-2-hydroxy-5-methyl-4-hexenoic acid (3aS,4R,7R,8aS,9S,10aR,12aS,12bR,13S,13aS)-7,12a-bis(acetyloxy)-13-(benzyloxy)-3a,4,7,8,8a,9,10,10a,12,12a,12b,13-dodecahydro-9-hydroxy-5,8a,14,14-tetramethyl-2,8-dioxo-6,13a-methano-13aH-oxeto[2″,3″:5′,6′]benzo[1′,2′:4,5]cyclodeca[1,2-d]-1,3-dioxol-4-yl ester,

d) BMS 188797 means

(2R,3S)-β-(benzoylamino)-α-hydroxy benzenepropanoic acid (2aR,4S,4aS,6R,9S,11S,12S,12aR,12bS)-6-(acetyloxy)-12-(benzoyloxy)-2a,3,4,4a,5,6,9,10,11,12,12a,12b-dodecahydro-4,11-dihydroxy-12b-[(methoxycarbonyl)oxy]-4a,8,13,13-tetramethyl-5-oxo-7,11-methano-1H-cyclodeca[3,4]benz[1,2-b]oxet-9-yl ester,

e) BMS 184476 means

(2R,3S)-β-(benzoylamino)-α-hydroxy benzenepropanoic acid (2aR,4S,4aS,6R,9S,11S,12S,12aR,12bS)-6,12b-bis(acetyloxy)-12-(benzoyloxy)-2a,3,4,4a,5,6,9,10,11,12,12a,12b-dodecahydro-11-hydroxy-4a,8,13,13-tetramethyl-4-[(methylthio)methoxy]-5-oxo-7,11-methano-1H-cyclodeca[3,4]benz[1,2-b]oxet-9-yl ester,

f) camptothecin means

4(S)-ethyl-4-hydroxy-1H-pyrano[3′,4′:6,7]indolizino[1,2-b]quinoline-3,14(4H,12H)-dione,

g) topotecan means

(4S)-10-[(dimethylamino)methyl]-4-ethyl-4,9-dihydroxy-1H-pyrano[3′,4′:6,7]indolizino[1,2-b]quinoline-3,14(4H,12H)-dione monohydrochloride,

h) DX-8951f means

(1S,9S)-1-amino-9-ethyl-5-fluoro-9-hydroxy-4-methyl-2,3,9,10,13,15-hexahydro-1H,12H-benzo[de]pyrano[3′,4′:6,7]indolizino[1,2-b]quinoline-10,13-dione,

i) BN-80915 means

5(R)-ethyl-9,10-difluoro-1,4,5,13-tetrahydro-5-hydroxy-3H,15H-oxepino[3′,4′:6,7]indolizino[1,2-b]quinoline-3,15-dione,

j) 9-aminocamptotecin means

(S)-10-amino-4-ethyl-4-hydroxy-1H-pyrano[3′,4′:6,7]indolizino[1,2-b]quinoline-3,14(4H,12H)-dione,

k) 9-nitrocamptothecin means

4(S)-ethyl-4-hydroxy-10-nitro-1H-pyrano[3′,4′,:6,7]-indolizino[1,2-b]quinolin-3,14(4H,12H)-dione,

l) DFDC means

2′-deoxy-2′,2′-difluorocytidine,

m) DMDC means

2′-deoxy-2′-methylidenecytidine,

n) FMDC means

(E)-2′-deoxy-2′-(fluoromethylene)cytidine,

o) Ara-C means

1-(β-D-arabinofuranosyl)cytosine,

p) decitabine means

4-amino-1-(2-deoxy-β-D-erythro-pentofuranosyl)-1,3,5-triazin-2(1H)-one,

q) troxacitabine refers to

4-amino-1-[(2S,4S)-2-(hydroxymethyl)-1,3-dioxolan-4-yl]-2(1H)-pyrimidinone,

r) fludarabine refers to

2-fluoro-9-(5-O-phosphono-β-D-arabinofuranosyl)-9H-purin-6-amine,

s) cladribine refers to

2-chloro-2′-deoxyadenosine,

t) dolastatin 10 means

N,N-dimethyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-L-valinamide,

u) dolastatin 14 means

cyclo[N-methylalanyl-(2E,4E,10E)-15-hydroxy-7-methoxy-2-methyl-2,4,10-hexadecatrienoyl-L-valyl-N-methyl-L-phenylalanyl-N-methyl-L-valyl-N-methyl-L-valyl-L-prolyl-N2-methylasparaginyl],

v) dolastatin 15 means

(1S)-1-[[(2S)-2,5-dihydro-3-methoxy-5-oxo-2-(phenylmethyl)-1H-pyrrol-1-yl]carbonyl]-2-methylpropyl ester N,N-dimethyl-L-valyl-L-valyl-N-methyl-L-valyl-L-prolyl-L-proline,

w) TZT 1027 means

N,N-dimethyl-L-valyl-N-[(1S,2R)-2-methoxy-4-[(2S)-2-[(1R,2R)-1-methoxy-2-methyl-3-oxo-3-[(2-phenylethyl)amino]propyl]-1-pyrrolidinyl]-1-[(1S)-1-methylpropyl]-4-oxobutyl]-N-methyl-L-valinamide,

x) cemadotin means

N,N-dimethyl-L-valyl-L-valyl-N-methyl-L-valyl-L-prolyl-N-(phenylmethyl)-L-prolinamide,

y) adriamycin means

(8S,10S)-10-[(3-amino-2,3,6-trideoxy-L-lyxo-hexopyranosyl)oxy]-7,8,9,10-tetrahydro-6,8,11-trihydroxy-8-(hydroxyacetyl)-1-methoxy-naphthacene-5,12-dione hydrochloride,

z) daunomycin means

8-acetyl-10-[(3-amino-2,3,6-trideoxy-L-lyxo-hexopyranosyl)oxy]-7,8,9,10-tetrahydro-6,8,11-trihydroxy-1-methoxy-naphthacene-5,12-dione, hydrochloride,

aa) idarubicin means

(7S,9S)-9-acetyl-7-[(3-amino-2,3,6-trideoxy-L-lyxo-hexopyranosyl)oxy]-7,8,9,10-tetrahydro-6,9,11-trihydroxy-naphthacene-5,12-dione,

bb) ZD 1839 means

N-(3-chloro-4-fluorophenyl)-7-methoxy-6-[3-(4-morpholinyl)propoxy]-4-quinazolmnamine,

cc) CP 358774 means

N-(3-ethynylphenyl)-6,7-bis(2-methoxyethoxy)-4-quinazolinamine,

dd) PD 158780 means

N⁴-(3-bromophenyl)-N6-methylpyrido[3,4-d]pyrimidine-4,6-diamine,

ee) GW 2016 means

N-(3-chloro-4-((3-fluorobenzyl)oxy)phenyl)-6-(5-(((2-methylsulfonyl)ethyl)amino)-methyl)-2-furyl)-4-quinazolinamine, and

ff) R 115777 refers to

6-[1-amino-1-(4-chlorophenyl)-1-(1-methylimidazol-5-yl)methyl]-4-(3-chlorophenyl)-1-methylquinolin-2(1H)-one.

In the present invention, enzymes that are preferably expressed in tumor tissue such that such enzymes effectively activate a therapeutic compound at the tumor tissue. Such therapeutic compounds are selectively identified by analyzing the levels of mRNAs and/or proteins of human tissue. Preferred therapeutic compounds are then designed from known and/or novel cytotoxic drugs by adding the moieties that mask the biological activities of the cytotoxic drugs but are recognized and removed by said enzymes selectively in targeting tumor tissue.

Yet another aspect of this invention is a method for creating an anti-cancer compound that is selectively activated in tumor tissues or cells. This method comprises comparing the level of gene or protein expression in tumor cells or tissues and normal cells or tissues; determining an enzyme capable of releasing an anti-cancer compound wherein the level of gene or protein expression in the tumor cells or tissues is at least twice the level of gene or protein expression in normal cells or tissues; and selecting a moiety that is selectively cleaved in the tumor cells or tissues by the enzyme releasing the anti-cancer compound.

### DETAILED DESCRIPTION OF THE INVENTION

The normal and tumorous human tissue used for the analyses include tissue from brain, esophagus, heart, lung, breast, stomach, liver, pancreas, gallbladder, small intestine, colon, rectum, kidney, bladder, ovary, uterus, testis, prostate, skin, bone, bone marrow, and blood. Preferably, as normal cells granulocyte progenitors are used to compare expression levels of genes and/or proteins between tumor and normal tissue and to select genes and/or proteins that are preferably expressed in tumor tissue. After human tissue is resected during surgeries, it is preferable that it is immediately frozen in liquid nitrogen or acetone containing dry ice with or without being embedded in O.C.T. compound (Sakura-Seiki, Tokyo, Japan, Catalog No. 4583) and stored at temperatures below −70 or −80° C. until use.

If the tumor tissue contains large portion of normal cells, tumor cells are isolated from the tissue that is embedded in OCT prodrugs by laser capture microdissection (Ohyama H, et al. Laser capture microdissection-generated target sample for high-density oligonucleotide array hybridization. Biotechniques 29, 530-536 (2000), Leethanakul C, et al., Gene expression profiles in squamous cell carcinomas of the oral cavity: use of laser capture microdissection for the construction and analysis of stage-specific cDNA libraries. Oral Oncol 36, 474-83 (2000)). For micirodissection, frozen sections of between 6 and 10 micro meter thickness are fixed with 70% ethanol, stained with Mayer's hematoxylin, and then dehydrated with ethanol gradient and xylene. Microdissection of tumor cells are performed by means of laser capture microdissection apparatus (Olympus, Tokyo, Japan, Model LM200), and the RNA in tumor cells is extracted using a commercially available kit (Micro RNA Isolation Kit, Stratagene, La Jolla, Calif., USA).

The human granulocyte progenitors that are most susceptible to cytotoxic drugs are prepared by expanding CD34-positive mononuclear cells on mouse stromal cells in the presence of several cytokines including Flt3-ligand, stem cell factor (SCF) and thrombopoietin (TPO). The CD34-positive mononuclear cells either in human umbilical cord blood or bone marrow are incubated with and bound to an anti-CD34 antibody that is conjugated with magnetic beads and purified by means of magnetic assisted cell sorting (MACS) (Miltenyi, et. al. In: Hematopoietic stem cells: The mulhouse mannual, 201-213, AlphaMed press, Dayton (1994)). The purified CD34-positive mononuclear cells that sustain abilities to differentiate into various types of hematopietic cells are expanded in culture dishes and the percentage of granulocyte progenitors in culture are confirmed by examining the expression of CD34 after staining the cells with a fluorescence-conjugated anti-CD 34 antibody. Usually, more than 90% of the cells in culture become CD34-positive granulocyte progenitors after expansion. The abilities of these granulocyte progenitors to differentiate into myeloblasts and then to myelocytes and granulocytes are tested by treating them with granulocyte colony stimulating factor (G-CSF) or interleukin-3 (IL3) in combination with granulocyte-macrophage colony stimulating factor (GM-CSF) and G-CSF. The cell lineage and stages of the differentiation are confirmed by monitoring the cell surface antigens (CD antigens) such as CD11, CD13, and CD15 by fluorescence assisted cell sorting (FACS) with FACSCalibur (Becton Dickinson, Franklin Lakes, N.J., USA) and/or by microscopy after staining the cells with Giemsa stain (Diff-Quick ) (Midori-Juji.Co. Osaka, Japan, Catalog No.16920) or Leishman stain (Merck, Darmstadt, Germany, Catalog No.1.05387.0500). FACS data is analyzed by FACSCalibur CELLQuest software according to the FACSCalibur mannual, FACStation ver.1.1 (Becton-Dickinson, Franklin Lakes, N.J., USA.).

Enzymes and proteins that are expressed in certain tumor tissue is searched by measuring their mRNAs and/or protein levels in human tissue and cells. Expression levels of mRNAs are determined by known methods such as DNA microarray (Schena, M. et al. Quantitative monitoring of gene expression patterns with a complementary DNA microarray. Science 270, 467-470 (1995), and Lipshutz, R. J. et al. High density synthetic oligonucleotide arrays. Nature Genetics 21, 20-24 (1999)), reverse transcription polymerase reaction (hereafter referred to as RT-PCR) (Weis, J. H. et al. Detection of rare mRNAs via quantitative RT-PCR, Trends Genetics 8, 263-264 (1992), and Bustin, S. A. Absolute quantification of mRNA using real-time reverse transcription polymerase chain reaction assays, J. Mol. Endocrinol. 25, 169-193 (2000)), northern blotting and in situ hybridization (Parker, R. M. & Barnes, N. M. mRNA: detection in situ and northern hybridization, Methods Mol. Biol. 106, 247-283 (1999)), RNase protection assay (Hod, Y. A. Simp lified ribonuclease protection assay, Biotechniques 13, 852-854 (1992), Saccomanno, C. F. et al. A faster ribonuclease protection assay, Biotechniques 13, 846-850 (1992)), western blotting (Towbin, H. et al. Electrophoretic transfer of proteins from polyacrylamide gels to nitrocellulose sheets, Proc. Natl. Acad. Sci. USA 76, 4350-4354 (1979), Burnette, W. N. Western blotting: Electrophoretic transfer of proteins form sodium dodecyl sulfate-polyacrylamide gels to unmodified nitrocellulose and radioiodinated protein A, Anal. Biochem. 112, 195-203 (1981)), ELISA assays (Engvall, E. & Perlman, P. Enzyme-linked immunosorbent assay (ELISA): Quantitative assay of immunoglobulin G, Immunochemistry 8: 871-879 (1971)), and protein arrays (Merchant, M. & Weinberger, S. R. Review: Recent advancements in surface-enhanced laser desorption/ionization-time of flight-mass spectrometry, Electrophoresis 21, 1164-1177 (2000), Paweletz, C. P. et al. Rapid protein display profiling of cancer progression directly from human tissue using a protein biochip, Drug Development Research 49, 34-42 (2000)). More preferably, DNA microarray and RT-PCR are used for high-throughput analysis and quantitative analysis of mRNA expression, respectively.

To perform DNA microarray, RNA is extracted from small pieces of tissue and/or cells that are rapidly frozen in liquid nitrogen or acetone-dry ice, and stored at temperature below −70° C. or −80° C. until use. Tissue and cells are homogenized, and RNAs in the tissue and cell homogenates are extracted with chloroform and precipitated with isopropyl alcohol. DNA contaminated in the RNA preparation is digested with DNase I, and the RNA is further purified by gel filtration column chromatography. Quality of the total RNA is judged from ratio of 28S and 18S ribosomal RNA after agarose gel electrophoresis and staining the RNA with ethidium bromide.

Using the total RNA as the template, cDNA is synthesized with an oligo-dT primer (Sawady Technology, Tokyo, Japan) that contained the sequences for the T7 promoter and reverse transcriptase. The resulting cDNA is extracted with the mixture of phenol and chloroform and is separated from short oligonucleotides by gel filtration column chromatography.

Using the cDNA as the template, cRNA is synthesized by using T7 polymerase, adenosine triphosphate (ATP), guanosine triphosphate (GTP), cytidine triphosphate (CTP), uridine triphosphate (UTP), Bio-11-CTP and Bio-16-UTP (ENZO Diagnostics, Farmingdale, USA, Catalog No. 42818 and 42814, respectively) at 37° C. for 6 hr. The resulting cRNA is separated from the nucleotides by gel filtration column chromatography. Quality of the cRNA is judged from the length of the cRNA after agarose gel electrophoresis and staining the cRNA with ethdium bromide.

DNA microarray is carried out with high-density oligonucleotide chips (HuGeneFL array, Affymetrix, Santa Clara, USA, Catalog No.510137) (Lipshutz, R. L. et al. Nature Genet. 21, 20-24 (1999)) according to the manufacture's instruction. Fragmentation of the cRNA at 95° C., hybridization and washing are performed according to the manufacturer's instruction. Each pixel level is collected with laser scanner (Affymetrix, Santa Clara, USA) and levels of the expression of each cDNA and reliability (Present/Absent call) are calculated with Affymetrix GeneChip ver.3.3 and Affymetrix Microarray Suite ver.4.0 softwares.

In addition to DNA microarrays, other methods including RT-PCR (Weis, J. H. et al. Detection of rare mRNAs via quantitative RT-PCR. Trends in Genetics, 8, 263-264 (1992), and Bustin, S. A. Absolute quantification of mRNA using real-time reverse transcription polymerase chain reaction assays. J. Molecular Endocrinology 25, 169-193 (2000)), northern blotting and in situ hybridization (Parker, R. M. and Barnes, N. M. mRNA: detection in situ and northern hybridization. Methods in Molecular Biology, 106, 247-283 (1999)), differential displays (Zhu, W. and Liang, P. Detection and isolation of differentially expressed genes by differential display. Methods Mol. Biol. 68, 211-20 (1997), Liang P. and Pardee A. B. Differential display of eukaryotic messenger RNA by means of the polymerase chain reaction. Science. 257, 967-971 (1992)), RNase protection assay (Hod, Y. A simplified ribonuclease protection assay. Biotechniques 13, 852-854 (1992), Saccomanno, C. F. et al. A faster ribonuclease protection assay. Biotechniques 13, 846-850 (1992)), protein arrays (Merchant, M. and Weinberger, S. R. Review: Recent advancements in surface-enhanced laser desorption/ionization-time of flight-mass spectrometry. Electrophoresis 21: 1164-1177 (2000), Paweletz, C. P. et al. Rapid protein display profiling of cancer progression directly from human tissue using a protein biochip. Drug Development Research 49: 34-42 (2000)), western blotting (Towbin, H. et al. Electrophoretic transfer of proteins from polyacrylamide gels to nitrocellulose sheets. Proc. Natl. Acad. Sci. USA 76: 4350-4354 (1979), Bumette, W. N. Western blotting: Electrophoretic transfer of proteins form sodium dodecyl sulfate-polyacrylamide. gels to unmodified nitrocellulose and radioiodinated protein A. Anal. Biochem. 112: 195-203 (1981)), two-demensional gel electrophoresis (O'Farrell, P. H. High-resolution two-dimentional electrophoresis of proteins. J. Biol. Chem. 250: 4007-4021 (1975)), ELISA assays (Engvall, E. and Perlman, P. Enzyme-linked immunosorbent assay (ELISA): Quantitative assay of immunoglobulin G. Immunochemistry 8: 871-879 (1971)) are also used to determine the levels of mRNAs and/or proteins.

Enzymes and/or proteins that are preferentially expressed in certain tumors but not in granulocyte progenitors and other normal tissue are identified by comparing the levels of mRNAs and proteins in tumor tissue with those in normal tissue. Genes and/or proteins whose expression levels differ by more than 2-fold between certain tumors and granulocyte progenitors are selected as the candidate genes for enzymes and/or proteins that are eligible for the activation of TTC. Genes and/or proteins showing bigger differences in the expression levels between certain tumors and granulocyte progenitors are more preferable. Thereafter, the levels of the mRNA that are highly expressed in certain tumor tissue but not in grannulocyte progenitors are compared with those in other normal tissue particularly with normal liver, because liver is the main organ that metabolizes most of drugs. The mRNA whose levels in certain tumor tissue are higher than those in hematopoietic progenitors and other normal tissue particularly in liver are selected.

Among the enzymes and/or proteins that are selected according to the differences in the expression levels between certain tumor tissue and granulocyte progenitors and other normal tissue such as liver, those with a relatively wide substrate specificity and an enzyme reaction mechanism suitable for a compound design are further selected.

Those enzymes include phospholipase C, microsomal dipeptidase, arylsulfatase A, DT-diaphorase, pyrroline 5′-carboxyreductase, dehydrodiol dehydrogenase, carbonylreductase, lysyl hydroxylase, prolidase, dihydropyrimidinase, glutamine:fructose-6-phosphate amidotransferase, UDP-galactose ceramide galactosyl transferase, lysyl oxidase, enolase, glucose-6-phosphate dehydrogenase, stearoyl-coenzyme A desaturase, epoxide hydrolase and aldolase C.

More preferable enzymes for TTC design are microsomal dipeptidase, phospholipase C, DT-diaphorase, dihydrodiol dehydrogenase, pyrroline 5′-carboxyreductase, carbonylreductase, lysyl hydroxylase or matrix metalloproteinases.

These enzymes can be utilized for designing anti-cancer compounds of the formula (I),

X—Y-Q (I)

wherein

X is a pro-moiety that is designed to generate an active anti-cancer substance (Q-Y—H) selectively in tumors by the enzymes discovered by the method of the present invention;

(Q-Y—) is a radical derived from the active anti-cancer substance (Q-Y—H) in which Y is —O—, —S— or —N—.

Compounds of formula (1) can be described in more detail as follows. An active anti-cancer substance, (Q-Y—H) can be any anti-tumor agents. They can be connected to a pro-moiety X through —Y—H group such as an primary or secondary amino, hydroxy, or sulfhydryl group in the structure of (Q-Y—H), in such a way that it can spontaneously release an active anti-cancer substance by the action of the enzyme(s) found by the methods of the present invention. More particularly, (Q-Y—H) is a cytotoxic agent such as a taxan, a camptothecin, an anti-cancer nucleoside, a dolastatin, and an anthracyclin and a farnesyltransferase inhibitor, an EGF receptor tyrosine kinase inhibitor and the like.

Preferred are compounds wherein the active anti-cancer substance (Q-Y—H) is a taxan selected from the group consisting of

a) taxol

[2aR-[2aα,4β,4aβ,6β,9α(αR*,βS*),11α,12α,12aα,12bα]]-β-(benzoylamino)-α-hydroxybenzenepropanoic acid 6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester,

b) taxotere

[2aR-[2aα, 4β,4aα, 6β,9α(αR*,βS*,11α, 12α, 12aα, 12bα)]-β-[[(1,1-dimethylethoxy)carbonyl]amino]-α-hydroxybenzenepropanoic acid 12b-(acetyloxy)-12-(benzoyloxy)-2a,3,4,4a,5,6,9,10,11,12,12a,12b-dodecahydro-4,6,11-trihydroxy-4a,8,13,13-tetramethyl-5-oxo-7,11-methano-1H-cyclodeca[3,4]benz[1,2-b]oxet-9-yl ester,

c) IDN 5109

(2R,3S)-3-[[(1,1-dimethylethoxy)carbonyl]amino]-2-hydroxy-5-methyl-4-hexenoic acid (3aS,4R,7R,8aS,9S,10aR,12aS,12bR,13S,13aS)-7,12a-bis(acetyloxy)-13-(benzloxy)-3a,4,7,8,8a,9,10,10a,12,12a,12b,13-dodecahydro-9-hydroxy-5,8a,14,14-tetramethyl-2,8-dioxo-6,13a-methano-13aH-oxeto[2″,3″:5′:,6′]benzo[1′,2′:4,5]cyclodeca[1,2-]-1,3-dioxol-4-yl ester,

d) BMS 188797

(2R,3S)-β-(benzoylamino)-α-hydroxy benzenepropanoic acid (2aR,4S,4aS,6R,9S,11S,12S,12aR,12bS)-6-(acetyloxy)-12-(benzoyloxy)-2a,3,4,4a,5,6,9,10,11,12,12a,12b-dodecahydro-4,11-dihydroxy-12b-[(methoxycarbonyl)oxy]-4a,8,13,13-tetramethyl-5-oxo-7,11-methano-1H-cyclodeca[3,4]benz[1,2-b]oxet-9-yl ester, and

e) BMS 184476

(2R,3S)-β-(benzoylamino)-α-hydroxy benzenepropanoic acid (2aR,4S,4aS,6R,9S,11S,12S,12aR,12bS)-6,12b-bis(acetyloxy)-12-(benzoyloxy)-2a,3,4,4a,5,6,9,10,11,12,12a,12b-dodecahydro-11-hydroxy-4a,8,13,1 3-tetramethyl-4-[(methylthio)methoxy]-5-oxo-7,11-methano-1H-cyclodeca[3,4]benz[1,2-b]oxet-9-yl ester.

Also preferred are compound wherein the active anti-cancer substance (Q-Y—H) is a camptothecin selected from the group consisting of

a) camptothecin

4(S)-ethyl-4-hydroxy-1H-pyrano[3′,4′:6,7]indolizino[1,2-b]quinoline-3,14(4H,12H)-dione,

b) topotecan

(4S)-10-[(dimethylamino)methyl]-4-ethyl-4,9-dihydroxy-1H-pyrano[3′,4′:6,7]indolizino[1,2-b]quinoline-3,14(4H,12H)-dione monohydrocbloride,

c) DX-8951f

(1S,9S)-1-amino-9-ethyl-5-fluoro-9-hydroxy-4-methyl-2,3,9,10,13,15-hexahydro-1H,12H-benzo[de]pyrano[3′,4′:6,7]indolizino[1,2-b]quinoline-10,13-dione,

d) BN-80915

5(R)-ethyl-9,10-difluoro-1,4,5,13-tetrahydro-5-hydroxy-3H,15H-oxepino[3′,4′:6,7]indolizino[1,2-b]quinoline-3,15-dione,

e) 9-aminocamptotecin

(S)-10-amino-4-ethyl-4-hydroxy-1H-pyrano[3′,4′:6,7]indolizino[1,2-b]quinoline-3,14(4H,12H)-dione,

f) 9-nitrocamptothecin

4(S)-ethyl-4-hydroxy-10-nitro-1H-pyrano[3′,4′,:6,7]-indolizino[1,2-b]quinoline-3,14(4H,12H)-dione,

g) (9S)-9-ethyl-9-hydroxy-1-pentyl-1H,12Hpyrano[3″,4″:6′,7′]indolizino-[1′,2′:6,5]pyrido[4,3,2-de]quinazoline-10,13(9H, 15H)-dione,

h) (9S)-9-ethyl-9-hydroxy-2-methyl-1-pentyl-1H,12Hpyrano[3″,4″:6′,7′]indolizino-[1′,2′:6,5]pyrido[4,3,2-de]quinazoline-10,13(9H,15H)-dione; and

i) (9S)-9-ethyl-9-hydroxy-2-hydroxymethyl-1-pentyl-1H,12H-pyrano[3″,4″:6′7′]indolizino[1′2′:6,5]pyrido[4,3,2-de]quinazoline-10,13(9H,15H)-dione.

Also preferred are compounds wherein the active anti-cancer substance (Q-Y—H) is an anti-cancer nucleoside selected from the group consisting of

a) DFDC

2′-deoxy-2′,2′-difluorocytidine,

b) DMDC

2′-deoxy-2′-methylidenecytidine,

c) FMDC

(E)-2′-deoxy-2′-(fluoromethylene)cytidine,

d) Ara-C

1-(β-D-arabinofuranosyl)cytosine,

e) decitabine

4-amino-1-(2-deoxy-β-D-erythro-pentofuranosyl)-1,3,5-triazin-2(1H)-one,

f) troxacitabine

4-amino-1-[(2S,4S)-2-(hydroxymethyl)-1,3-dioxolan-4-yl]-2(1H)-pyrimidinone,

g) fludarabine

2-fluoro-9-(5-O-phosphono-β-D-arabinofuranosyl)-9H-purin-6-amine, and

h) cladribine

2-chloro-2′-deoxyadenosine.

Also preferred is a compound wherein the active anti-cancer substance Q-Y—H is a dolastatin selected from the group consisting of

a) dolastatin 10

N,N-dimethyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-L-valinamide,

b) dolastatin 14 2,4,10-hexadecatrienoyl-L-valyl-N-methyl-L-phenylalanyl-N-methyl-L-valyl-N-methyl-L-valyl-L-prolyl-N2-methylasparaginyl],

c) dolastatin 15

(1S)-1-[[(2S)-2,5-dihydro-3-methoxy-5-oxo-2-(phenylmethyl)-1H-pyrrol-1-yl]carbonyl]-2-methylpropyl ester N,N-dimethyl-L-valyl-L-valyl-N-methyl-L-valyl-L-prolyl-L-proline,

d) TZT 1027

N,N-dimethyl-L-valyl-N-[(1S,2R)-2-methoxy-4-[(2S)-2-[(1R,2R)-1-methoxy-2-methyl-3-oxo-3-[(2-phenylethyl)amino]propyl]-1-pyrrolidinyl]-1-[(1S)-1-methylpropyl]-4-oxobutyl]-N-methyl-L-valinamide, and

e) cemadotin

N,N-dimethyl-L-valyl-L-valyl-N-methyl-L-valyl-L-prolyl-N-(phenylmethyl)-L-prolinamide.

Also preferred is a compound wherein the active anti-cancer substance (Q-Y—H) is an anthracycline selected from the group consisting of

a) adriamycin

(8S,10S)-10-[(3-amino-2,3,6-trideoxy-L-lyxo-hexopyranosyl)oxy]-7,8,9,10-tetrahydro-6,8,11-trihydroxy-8-(hydroxyacetyl)-1-methoxy-naphthacene-5,12-dione hydrochloride,

b) daunomycin

8-acetyl-10-[(3-amino-2,3,6-trideoxy-L-lyxo-hexopyranosyl)oxy]-7,8,9,10-tetrahydro-6,8,11-trihydroxy-1-methoxy-naphthacene-5,12-dione, hydrochloride, and

c) idarubicin

(7S,9S)-9-acetyl-7-[(3-amino-2,3,6-trideoxy-L-lyxo-hexopyranosyl)oxy]-7,8,9,10-tetrahydro-6,9,11-trihydroxy-naphthacene-5,12-dione.

Also preferred is a compound wherein the active anti-cancer substance (Q-Y—H) is EGF a recepter tyrosin kinase inhibitor or a farnesyltransferase inhibitor. Also preferred is a compound wherein the active anti-cancer substance (Q-Y—H) is an EGF recepter tyrosinkinase inhibitor selected from the group consisting of

a) ZD 1839

N-(3-chloro-4-fluorophenyl)-7-methoxy-6-[3-(4-morpholinyl)propoxy]-4-quinazolinamine,

b) CP 358774

N-(3-ethynylphenyl)-6,7-bis(2-methoxyethoxy)-4-quinazolinamine,

c) PD 158780

N⁴-(3-bromophenyl)-N6-methylpyrido[3,4-d]pyrimidine-4,6-diamine, and

d) GW 2016

N-(3-chloro-4-((3-fluorobenzyl)oxy)phenyl)-6-(5-(((2-methylsulfonyl)ethyl)amino)methyl)-2-furyl)-4-quinazolinamine.

Also preferred is a compound wherein the active anti-cancer substance (Q-Y—H) is the farnesyltransferase inhibitor R 115777 of the formula 6-[1-amino-1-(4-chlorophenyl)-1-(1-methylimidazol-5-yl)methyl]-4-(3-chlorophenyl)-1-methylquinolin-2(1H)-one.

Tumor targeting compounds of the formula (II) of the present invention,

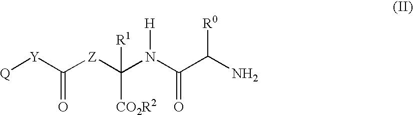

wherein

Q and Y are the same as defined above; R⁰ is a side chain of natural or non-natural amino acid; Z is (C1-C3)alkylene or —O—CH(R³)— wherein R³ is hydrogen or straight (C1-C4) alkyl, R¹ is hydrogen or methyl; R² is hydrogen, branched (C3-C10)alkyl or (C3-C8)cycloalkyl, which generate an active anti-cancer substances selectively in tumor by an action of microsomal dipeptidase are exemplified below as an example of compound design using an enzyme found by the methods described above. But these are not intended to limit the scope of the invention thereto. Compounds of formula (II) also include pharmaceutically acceptable salts thereof.

In the present invention, the first example of tumor targeting compounds designed with taxans as active anti-cancer drugs and microsomal dipeptidase as an activation enzyme is depicted as the general formula (III),

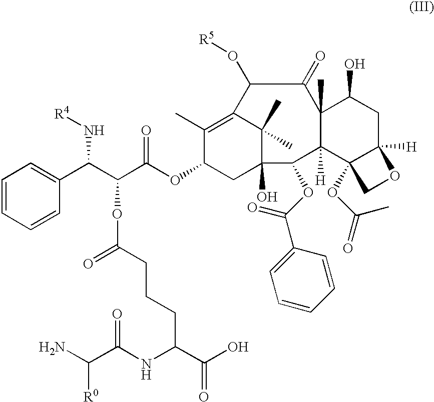

wherein

R⁰ is the same as defined above, R⁴ is benzoyl or tert-butoxycarbonyl, and R⁵ is hydrogen or acetyl and pharmaceutical acceptable salts thereof.

The preferable embodiments of R⁰ in the formula (III) are methyl, isopropyl, 2-methylpropyl, 1-methylpropyl, benzyl, indol-3-ylmethyl, and 2-(methylthio)ethyl; more preferably methyl, benzyl, and 2-methypropyl.

Preferred compounds of the formula (III) in accordance with the present invention are as follows:

a) 13-((2R,3S)-2-{(5S)-[5-((2S)-2-amino-4-methyl-pentanoylamino)-5-hydroxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one,

b) 13α-((2R,3S)-2-{(5S)-[5-((2S)-2-amino-propinoylamino)-5-hydroxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one, and

c) 13-((2R,3S)-2-{(5S)-[5-((2S)-2-amino-3-phenyl-propinoylamino)-5-hydroxycarbonyl]pentanoyloxy}-3-benzoy]amino-3-phenylpropionyloxy)-2α-benzyloxy-4α,10β-diacetoxy-1β,7β-dihydroxy-5β,20-epoxy-tax-11-en-9-one,

and pharmaceutically acceptable salts thereof.

The tumor selective activation of the compounds of the formula (III) by microsomal dipeptidase is illustrated in FIG. 1.

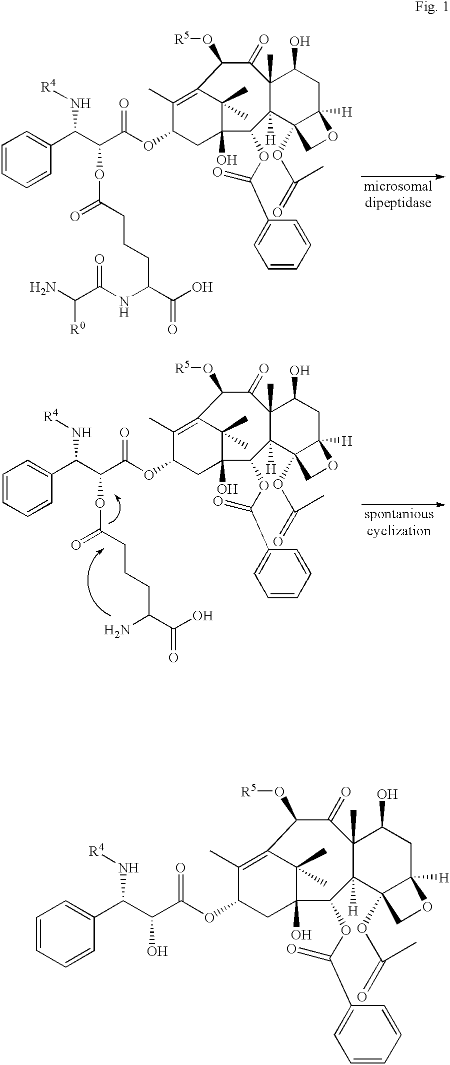

The second example of tumor targeting compounds designed with nucleoside derivative as an active anti-cancer drug and microsomal dipeptidase as an activation enzyme is depicted in the formula (IV),

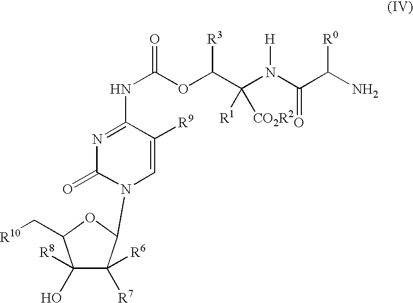

wherein

R⁰, R¹, R² and R³ are the same as defined in the formula (II), R⁶ is hydrogen, fluorine, hydroxyl or cyano, R⁷ is hydrogen, fluorine or hydroxy or R⁶ and R⁷ taken together form methylidene or fluoromethylidene, R⁸ is hydrogen or ethynyl, R⁹ is hydrogen, fluorine, vinyl or ethynyl, and R¹⁰ is hydrogen or hydroxy, and pharmaceutically acceptable salts thereof.

A preferred embodiment of the invention relates to compounds of formula (IV) as defined above wherein R⁶ is a hydrogen, fluorine, hydroxyl, R⁷ is a fluorine or hydroxy or R⁶ and R⁷ taken together form a methylidene or fluoromethylidene group. Another preferred embodiment of the invention relates to the above compound of formula (IV), wherein R⁰ is 2-methylpropyl, cyclohexylmethyl, 2-naphtylmethyl, 4-phenylbenzyl, (4-cyclohexylcyclo-hexyl)methyl, alkylthiomethyl, cyclohexylthiomethyl or 4-alkoxybenzyl, and R³ is hydrogen or methyl.

The preferred embodiment of active nucleosides containing in the formula (IV) is DFDC, DMDC, FMDC, Ara-C, decitabine, troxacitabine, 2′-cyano-2′-deoxycytidine, 3′-ethynylcytidine, 5-fluoro-5′-deoxycytidine, 5-viny-5′-deoxycytidine and the like; more preferably DFDC, DMDC and FMDC.

The preferred embodiment of R⁰ in the formula (IV) is the residue of lipophilic natural amino acid, (C8-C12)alkyl, (C3-C8)cycloalkylmethyl, substituted or unsubstituted benzyl or naphtylmetyl, (C8-C12)alkylthiomethyl, (C3-C8)cycloalkylthiomethyl, more preferably 2-methylpropyl, cyclohexylmethyl, benzyl, napht-2-ylmethyl, 4-phenylbenzyl, methylthioethyl, cyclohexylthiomethyl and the like.

Preferred compounds of the formula (IV) in accordance with the present invention may be selected from the group consisting of:

a) (2R)-((2S)-amino-3-cyclohexyl-propionylamino)-(3S)-[1-((4S)-hydroxy-(5R)-hydroxymethyl-3-methylene-tetrahydro-furan-(2R)-yl)-2-oxo-1,2-dihydro-pyrimidine-4-ylcarbamoyloxy]-butyric acid,

b) (2R)-((2S)-Amino-4-methyl-pentanoylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid,

c) (2R)-((2S)-Amino-3-biphenyl-4-yl-propionylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid,

d) 2(R)-[2(S)-Amino-3-biphenyl-4-yl-propionylamino]-3-{1-[4(S)-hydroxy-5(R)-hydroxymethyl-3-methylene-tetrahydro-furan-2(R)-yl]-2-oxo-1,2-dihydro-pyrimidin-4-ylcarbamoyloxy}-propionic acid,

e) (2R)-((2S)-Amino-3-naphthalen-2-yl-propionylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid,

f) (2R)-{(2S)-Amino-3-[4-(4-hydroxy-phenoxy)-phenyl]-propionylamino}-3-[1-((4S)-hydroxy-(5R)-hydroxymethyl-3-methylene-tetrahydro-furan-2-yl)-2-oxo-1,2-dihydro-pyrimidin-4-ylcarbamoyloxy]-butyric acid,

g) (2R)-[(2S)-amino-3-(4-methoxy-phenyl)-propionylamino]-(3S)-[1-[(4S)-hydroxy-(5R)-hydroxymethyl-3-methylene-tetrahydro-furan-2-yl]-2-oxo-1,2-dihydro-pyrimidin-4-ylcarbamoyloxy]-butyric acid,

h) (2R)-[(2S)-Amino-4-ethylsulfanyl-butyrylamino]-(3S)-[1-[(4S)-hydroxy-(5R)-hydroxymethyl-3-methylene-tetrahydro-furan-(2R)-yl]-2-oxo-1,2-dihydro-pyrimidin-4-ylcarbamoyl]-butyric acid,

i) (2R)-((2S)-Amino-3-cyclohexyl-propionylamino)-(3S)-[1-(3,3-difluoro-(4R)-hydroxy-(5R)-hydroxymethyl-tetrahydro-furan-2-yl)-2-oxo-1,2-dihydro-pyrimidin-4-ylcarbamoyloxy]-butyric acid,

j) 2(S)-[2(S)-amino-3-cyclohexyl-propionylamino)-3-[1-(3,3-difluoro-4(R)-hydroxy-5(R)-hydroxymethyl-tetrahydro-furan-2(R)-yl)-2-oxo-1,2-dihydro-pyrimidin-4-ylcarbamoyloxy]-2(S)-methyl-propionic acid,

k) 2(R)-[2(S)-amino-3-cyclohexyl-propionylamino]-3-{1-[3,3-difluoro-4(R)-hydroxy-5(R)-hydroxymethyl-tetrahydro-furan-2(R)-yl]-2-oxo-1,2-dihydro-pyrimidin-4-ylcarbamoyloxy}-2(R)-methyl-propionic acid,

l) (2S,3S)-2-(2-amino-3-cyclohexyl-propionylamino)-3-[1-{(4R,5R)-3,3-difluoro-4-hydroxy-5-hydroxylmethyl-tetrahydro-furan-2-yl}-2-oxo-1,2-dihydro-pyridine-4-ylcarbamoyloxy]-2-methyl-butyric acid,

m) (2R,3R)-2-(2-amino-3-cyclohexyl-propionylamino)-3-[1-{(4R,5R)-3,3-difluoro-4-hydroxy-5-hydroxylmethyl-tetrahydro-furan-2-yl}-2-oxo-1,2-dihydro-pyridine-4-ylcarbamoyloxy]-2-methyl-butyric acid, and

n) (2R)-[(2S)-amino-3-cyclohexyl-propionylamino]-(3S)-[1-[(4S)-hydroxy-(5R)-hydroxymethyl-3-methylene-tetrahydro-furan-(2R)-yl]-2-oxo-1,2-dihydro-pyrimidine-4-ylcarbamoyloxy]-butyric acid isopropyl ester, and

pharmaceutically acceptable salts thereof.

The third example of tumor targeting compounds designed with nucleosides as active anti-cancer drugs and microsomal dipeptidase as an activation enzyme is depicted in the formula (V),

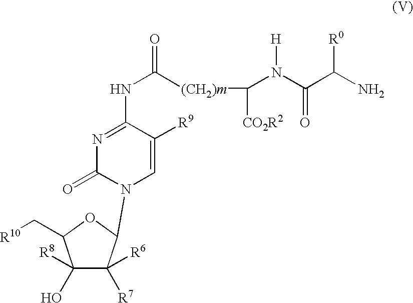

wherein

m is an integer of 2 or 3, and R⁰, R², R⁶, R⁷, R⁸, R⁹ and R¹⁰ are the same as defined above.

The preferred embodiment of active cytidine analogs containing in the formula (V) is DFDC, DMDC, FMDC, Ara-C, decitabine, troxacitabine, 2′-cyano-2′-deoxycytidine, 3′-ethynylcytidine, 5-fluoro-5′-deoxycytidine, 5-viny-5′-deoxycytidine and the like; more preferably DFDC, DMDC, and FMDC.

The preferred embodiment of R⁰ in the formula (V) is cyclohexylmethyl, napht-2-ylmethyl, 4-phenylbenzyl, benzyl, indol-3-ylmethyl or 4-alkoxybenzyl, e.g. (4-lower-alkoxyphenyl)methyl such as 4-methoxybenzyl, 4-ethoxybenzyl and the like.

Preferred compounds of formula (V) in accordance with the present invention are as follows:

a) (2R)-[(2S)-amino-3-(1H-indol-3-yl)propionylamino]-4-[1-((4S)-hydroxy-(5R)-hydroxymethyl-3-methylenetetrahydrofuran-2-yl)-2-oxo-1,2-dihydropyrimidin-4-ylcarbamoyl]-butyric acid,

b) (2R)-((2S)-amino-3-cyclohexylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid,

c) (2R)-((2S)-amino-3-biphenyl-4-ylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid, and

d) (2R)-((2S)-amino-3-naphthalen-2-ylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid,

and pharmaceutically acceptable salts thereof.

Another example of tumor targeting compounds designed with camptothecins as active anti-cancer drugs and microsomal dipeptidase as an activation enzyme is depicted in the formula (VI),

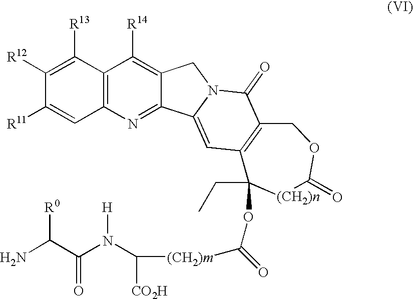

wherein

m is an integer of 1 to 3, n is an integer of 0 to 1, R⁰ is the same as defined in the formula (II), R¹¹ is hydrogen or fluorine, R¹² is hydrogen, fluorine, methyl or hydroxy, R¹³ is hydrogen, amino, nitro, or (di-methylamino)methyl, R¹⁴ is hydrogen, (C1-C4)alkyl, (4-methylpiperazinyl)methyl, (tert-butoxyimino)methyl or R¹³ and R¹⁴, or R¹¹ and R¹² taken together form a 05 or 6 membered ring which optionally contain 1 or 2 hetero atom(s) and may be optionally substituted with 1 to 3 substituant(s) selected from the group consisting of (C1-C8)alkyl, amino, (C1-C8)alkylamino and di-(C1-C4)alkylamino and pharmaceutcially acceptable salts thereof. More preferable, the compounds of formula (VI) are characterized by R¹¹ being hydrogen, R¹² being hydrogen or hydroxy, R¹³ being hydrogen or (dimethylamino)methyl and R¹⁴ being hydrogen or ethyl. The preferred embodiment of R⁰ in the formula (VI) is 2-methylpropyl, cyclohexylmethyl, benzyl, indol-3-ylmethyl, 4-aminobutyl, 4-aminopropyl; more preferably 2-methylpropyl, cyclohexylmethyl, benzyl and indol-3-ylmethyl.

The preferred embodiments of active camptohecin analog containing in the formula (VI) are camptothecin, topotecan, SN-38, lurtotecan, 9-aminocamptotecin, 9-nitrocamptothecin, DX-8951f, BN-80915, (9S)-9-ethyl-9-hydroxy-1-pentyl-1H,12H-pyrano[3″,4″:6′,7′]indolizino[1′,2′:6,5]pyrido[4,3,2-de]quinazoline-10,13(9H,15H)-dione, 9S)-9-ethyl-9-hydroxy-2-methyl-1-pentyl-1H,12Hpyrano[3″,4″:6′,7′]indolizino[1′,2′:6,5]-pyrido[4,3,2-de]quinazoline-10,13(9H,15H)-dione and (9S)-9-ethyl-9-hydroxy-2-hydroxymethyl-1-pentyl-1H,12H-pyrano[3″,4″:6′7′]indolizino[1′2′:6,5]pyrido[4,3,2-de]quinazoline-10,13(9H,15H)-dione and the like.

The preferred embodiment of R⁰ in the formula (VI) is 2-methylpropyl, cyclohexylmethyl, benzyl, indol-3-ylmethyl, 4-aminobutyl, 4-aminopropyl; more preferably 2-methylpropyl, cyclohexylmethyl, benzyl and indol-3-ylmethyl.

Preferred compounds of the formula (VI) in accordance with the present invention are as follows:

a) 20-O-[(S)-tryptophyl-γ-(S)-glutamyl]-20-(S)-camptothecin,

b) 20-O-[(S)-valyl-γ-(S)-glutamyl]-20(S)-camptothecin,

c) 20-O-[(S)-phenylalanyl-γ-(S)-glutamyl]-20(S)-camptothecin,

d) 20-O-[(S)-leucyl-γ-(S)-glutamyl]-20(S)-camptothecin,

e) 20-O-[(R)-leucyl-γ-(S)-glutamyl]-20(S)-camptothecin,

f) 20-O-[(R)-phenylalanyl-γ-(S)-glutamyl]-20(S)-camptothecin,

g) 20-O-[(S)-tryptophyl-γ-(R)-glutamyl]-20(S)-camptothecin,

h) 20-O-[(R)-tryptophyl-γ-(R)-glutamyl]-20(S)-camptothecin,

i) 20-O-[(S)-phenylalanyl-γ-(R)-glutamyl]-20(S)-camptothecin,

j) 20-O-[(S)-leucyl-γ-(R)-glutamyl]-20(S)-camptothecin,

k) 20-O-[(R)-tryptophyl-γ-(S)-glutamyl]-20(S)-camptothecin,

l) 20-O-[(R)-phenylalanyl-γ-(R)-glutamyl]-20(S)-camptothecin,

m) 20-O-[(R)-leucyl-γ-(R)-glutamyl]-20(S)-camptothecin,

n) 7-ethyl-10-hydroxy-20-O-[(R)-tryptophyl-(R)-homoglutamyl]-20(S)-camptothecin,

o) 7-ethyl-10-hydroxy-20-O-[(R)-tryptophyl-γ-(R)-glutamyl]-20(S)-camptothecin,

p) 7-ethyl-10-hydroxy-20-O-[(S)-phenylalanyl-γ-(R)-glutamyl]-20(S)-camptothecin,

q) 7-ethyl-10-hydroxy-20-O-[(S)-phenylalanyl-γ-(S)-aspartyl]-20(S)-camptothecin,

r) 7-ethyl-10-hydroxy-20-O-[(S)-leucyl-γ-(S)-aspartyl]-²⁰(S)-camptothecin,

s) 20-O-[(S)-tryptophyl-β-(R)-aspartyl]-20(S)-camptothecin,

t) 20-O-[(S)-phenylalanyl-β-(R)-aspartyl]-20(S)-camptothecin,

u) 20-O-[(R)-phenylalanyl-β-(R)-aspartyl]-20(S)-camptothecin,

v) 20-O-[(S)-phenylalanyl-β-(S)-aspartyl]-20(S)-camptothecin,

w) 20-O-[(S)-leucyl-β-(R)-aspartyl]-20(S)-camptothecin,

x) 20-O-[(S)-valyl-β-(R)-aspartyl]-20(S)-camptothecin,

y) 7-ethyl-10-hydroxy-20-O-[(S)-cyclohexylalanyl-(R)-glutamyl]-20(S)-camptothecin,

x) 7-ethyl-10-hydroxy-20-O-[(S)-cyclohexylalanyl-(S)-glutamyl]-20(S)-camptothecin,

aa) 20-O-[(S)-lysyl-γ-(S)-glutamyl]-20-(S)-camptothecin, and

bb) 20-O-[(S)-ornithyl-γ-(S)-glutamyl]-20-(S)-camptothecin,

cc) (9S)-9-ethyl-9-[(L)-tryptophyl-(L)-γ-glutamyloxy]-pentyl-1H,12H-pyrano[3″,4″:6′,7′]indolizino[1′,2′:6,5]pyrido[4,3,2-de]quinazoline-10,13(9H,15H)-dione hydrochloride,

dd) (9S)-9-ethyl-9-[(L)-cyclohexylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

ee) (9S)-9-ethyl-9-[(L)-phenylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

ff) (9S)-9-ethyl-9-[(L)-leucyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

gg) (9S)-9-ethyl-9-[(L)-lysyl-(L)-γ-glutamyloxy]-1-pentyl-1H,12H-pyrano[3″,4″:6′,7′]indolizino[1′,2′:6,5]pyrido[4,3,2-de]quinazolin-10,13(9H,15H)-dione dihydrochloride,

hh) (9S)-9-ethyl-9-[(L)-valyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

ii) (9S)-9-ethyl-9-[(L)-ornithyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

jj) (9S)-9-ethyl-9-[(L)-leucyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione methanesulfonic acid salt,

kk) (9S)-9-ethyl-9-[(D)-lysyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

ll) (9S)-9-ethyl-9-[(L)-phenylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

mm) (9S)-9-ethyl-9-[(L)-cyclohexylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

nn) (9S)-9-ethyl-9-[(L)-cyclohexylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

oo) (9S)-9-ethyl-9-[(L)-tryptophyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

pp) (9S)-9-ethyl-9-[(L)-ornithyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

qq) (9S)-9-ethyl-9-[(L)-leucyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

rr) (9S)-9-ethyl-9-[(L)-valyl-(D)-β-aspartyloxy]-1-pentyl-1H,12H-pyrano[3″,4″:6′,7′]indolizino[1′,2′:6,5]pyrido[4,3,2-de]quinazoline-10, 13(9H,15H)-dione hydrochloride,

ss) (9S)-9-ethyl-9-[(L)-leucyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

tt) (9S)-9-ethyl-9-[(L)-cyclohexylglycyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

uu) (9S)-9-ethyl-9-[(D)-cyclohexylalanyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

vv) (9S)-9-ethyl-9-[(L)-lysyl-(D)-γ-glutamyloxy]-1-pentyl-1H, 12H-pyrano[3″,4″:6′,7′]indolizino[1′,2′:6,5]pyrido[4,3,2-de]quinazoline-10,13(9H,15H)-dione dihydrochloride,

ww) (9S)-9-ethyl-9-[(L)-tryptophyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

xx) (9S)-9-ethyl-9-[(L)-leucyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

yy) (9S)-9-ethyl-9-[glycyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

zz) (9S)-9-ethyl-9-[(L)-alanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

aaa) (9S)-9-ethyl-9-[(L)-phenylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

the salt free compounds and other pharmaceutically acceptable salts thereof.

More preferable embodiments of the compounds of the formula (VI) are as follows:

a) 20-O-[(S)-tryptophyl-γ-(S)-glutamyl]-20(S)-camptothecin,

b) 20-O-[(S)-leucyl-γ-(S)-glutamyl]-20(S)-camptothecin,

c) 20-O-[(S)-tryptopbyl-γ-(R)-glutamyl]-20(S)-camptothecin,

d) 20-O-[(S)-leucyl-γ-(R)-glutamyl]-20(S)-camptothecin,

e) 7-ethyl-10-hydroxy-20-O-[(S)-phenylalanyl-β-(R)-glutamyl]-20(S)-camptothecin,

f) 7-ethyl-10-hydroxy-20-O-[(S)-phenylalanyl-β-(S)-aspartyl]-20(S)-camptothecin,

g) 20-O-[(S)-phenylalanyl-β-(S)-aspartyl]-20(S)-camptothecin,

h) 7-ethyl-10-hydroxy-20-O-[(S)-cyclohexylalanyl-(R)-glutamyl]-20(S)-camptothecin,

i) 7-ethyl-10-hydroxy-20-O-[(S)-cyclohexylalanyl-(S)-glutamyl]-20(S)-camptothecin,

j) 9S)-9-ethyl-9-[(L)-tryptophyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

k) (9S)-9-ethyl-9-[(L)-cyclohexylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

l) (9S)-9-ethyl-9-[(L)-phenylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

m) (9S)-9-ethyl-9-[(L)-leucyl-(D)-γ-glutamyloxy]-1-pentyl-1H,12H-pyrano[3″,4″:6′,7′]indolizino[1′,240 :6,5]pyrido[4,3,2-de]quinazoline-10,13(9H,15H)-dione hydrochloride,

n) (9S)-9-ethyl-9-[(L)-lysyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

o) (9S)-9-ethyl-9-[(L)-valyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

p) (9S)-9-ethyl-9-[(L)-omithyl-(L)-γ-glutamyloxy]-1-pentyl-1H,l12H-pyrano[3″,4″:6′,7′]indolizino[1′,2′:6,5]pyrido[4,3,2-de]quinazoline-10,13(9H,15H)-dione dihydrochloride,

q) (9S)-9-ethyl-9-[(L)-leucyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione methanesulfonic acid salt,

r) (9S)-9-ethyl-9-[(D)-lysyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

s) (9S)-9-ethyl-9-[(L)-phenylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

t) (9S)-9-ethyl-9-[(L)-cyclohexylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

u) (9S)-9-ethyl-9-[(L)-cyclohexylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

v) (9S)-9-ethyl-9-[(L)-tryptophyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

w) (9S)-9-ethyl-9-[(L)-ornithyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

x) (9S)-9-ethyl-9-[(L)-leucyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

y) (9S)-9-ethyl-9-[(L)-valyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

z) (9S)-9-ethyl-9-[(L)-leucyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

aa) (9S)-9-ethyl-9-[(L)-cyclohexylglycyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

bb) (9S)-9-ethyl-9-[(D)-cyclohexylalanyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

cc) (9S)-9-ethyl-9-[(L)-lysyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,

dd) (9S)-9-ethyl-9-[(L)-tryptophyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

ee) (9S)-9-ethyl-9-[(L)-leucyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

ff) (9S)-9-ethyl-9-[glycyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

gg) (9S)-9-ethyl-9-[(L)-alanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

hh) (9S)-9-ethyl-9-[(L)-phenylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,

the salt free compounds and other pharmaceutically acceptable salts thereof.

The most preferred embodiment of the compounds of the formula (VI) is (9S)-9-ethyl-9-[(L)-lysyl-(L)-γ-glutamyloxy]-1-pentyl-1H,12H-pyrano[3″,4″:6′,7′]indolizino-[1′,2′:6,5]pyrido[4,3,2-de]quinazoline-10,13(9H,15H)-dione dihydrochloride, the salt free compound and its pharmaceutically acceptable salts thereof.

The compound of formula (I) may be prepared by condensation reaction of a compound Q-Y—H with a reactive derivative of X. Thesc reactions are known in the art: e.g., the compound of the formula (II), (III), (V) and (VI) can be prepared by condensation reaction of the compound of formula (VII), and the compound of the formula (IV) can be prepared by condensation reaction of the compound of formula (VIII) as described below.

The compound of the formula (II), (III), (V) and (VI) can be prepared by condensation reaction of the compound of formula (VII),

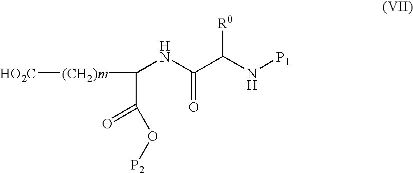

wherein

P₁ and P₂ are amino and carboxy protecting groups respectively; R⁰, and m are the same as defined above, and suitably protected by an anti-cancer substance such as paclitaxel, cytidine derivatives or camptothecins with a condensation agent, such as dicyclohexylcarbodiimide, BOP, HBTU, TNTU, PyBroP™, PyBOP™, TBTU, TSTU, HOBt [commercially available coupling reagents: cf. The Combinatorial Chemistry Catalog, February, 1997; Novabiochem.] and the like, followed by removal of protecting group(s).

In the above, amino and carboxy protecting groups P1 and P2, as well as the condensation reaction are known to those skilled in the art. [cf. The practice of Peptide Synthesis, M. Bodansky and A. Bodansky/2^{nd} ed., 1994 (Springer-Verlag)].

The compound of the formula (IV) can be prepared by condensation reaction of the compound of formula (VIII),

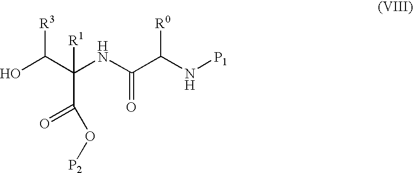

wherein

P₁, P₂, R⁰, R¹, and R³ are the same as defined above, and a suitably protected cytidine derivative with a condensation agent such as 4-nitrophenyl chloroformate and triphosgene, followed by removal of protecting group(s).

The reaction can be carried out in a solvent such as methylene dichloride, pyridine, N,N-dimethylformamide, N-methylpyrrolidone, acetonitrile and the like in the presence or absence of base such as triethylamine, di-isopropylethylamine, pyridine, N,N-dimethylaminopyridine and the like at a temperature between −20° C. and +50° C., preferably at 0° C. to +25° C.

The removal of the amino protecting group, when using amino and/or carboxy-protected dipeptide for the condensation reaction, can be done by the method known to those skilled in the art, e.g., treatment with trifluoroacetic acid for Boc group, piperidine for Fmoc group, or tetrabutylammonium fluoride for 2-(trimethylsilyl)ethoxycarbonyl (Teoc), trimethylsilylethyl and ter-butyldimethylsilyl group, and catalytic hydrogenolysis for Cbz group.

The amino acid derivatives used for the preparation of the dipeptide derivatives in the formula (VII) and (VIII) are either commercially available or prepared by the known methods described in the literatures (e.g. J. Am. Chem. Soc. 2000, 122, 762-766; J. Org. Chem. 1998 5240; Tetrahedron Asymmetry 1995, 1741; Tetrahedron Asymmetry 1998, 4249). S-Alkyl-cystein derivatives were prepared either by S-alkylation of amino/carboxy-protected cysteine derivatives with an alkylating agent, or replacement of the hydroxy group of amino/carboxy-protected serine derivatives with bromine atom followed by substitution reaction with a thiol derivative. O-Alkyl-tyrosine derivatives were prepared by O-alkylation of amino/carboxy-protected tyrosine derivatives with an alkylating agent.

These dipeptide derivatives can be prepared by the conventional peptide chemistry known to those skilled in the art [cf. The practice of Peptide Synthesis, M. Bodansky and A. Bodansky/2^{nd} ed., 1994 (Springer-Verlag)]

TTCs are then tested for their selective activation by a certain enzyme using the recombinant enzymes and/or the extracts of cells that are expressing or not expressing high levels of TTC-activating enzymes. The human hematopoietic progenitors are also used as cells that do not express or express only low levels of TTC-activating enzymes. Recombinant proteins for TTC-activating enzymes can be generated by expressing cDNAs for the enzymes in bacteria or other cells including insect cells and mammalian cells. Cell lines that constitutively express high levels of the TTC-activating enzymes are also generated by transfecting the plasmid in which a cDNA for a TYC-activating enzyme is cloned downstream of a strong the constitutive promoter including the cytomegalo virus (CMV) promoter (Foecking, M. K. and Hofstetter, H. Powerful and versatile enhancer-promoter unit for mammalian expression vectors. Gene. 45, 101-105 (1986)). Thus, the transcription of the TTC-activating enzymes in the transfectants is under the control of a strong constitutive promoter. Activation of TTCs is examined by incubating TTCs with the recombinant TTC-activating enzymes and/or cell extracts that are expressing or not expressing a TTC-activation enzyme and by measuring the amounts of TTCs and active drugs by HPLC and/or LCMS.

In addition to the cells bearing the additional copies of the cDNAs for the TTC-activating enzymes, extracts of various tissue of human and animals are also used to confirm the tumor specific activation of TTCs. Tumorous and normal tissue used for the analysis includes tissue from brain, heart, lung, stomach, intestine, colon, liver, kidney, blood and bone marrow from mice, rats, monkeys and humans.

Selective action of TTCs is further confirmed by comparing the growth inhibition of cells by TTCs between the cells expressing high levels of a TTC-activating enzymes and those expressing very low levels of the TTC-activating enzyme. Growth inhibition of cells is determined by quantifying the living cells after cultivating the cells in the presence or absence of TTCs.

The activation of the compounds is mediated by microsomal dipeptidase. The inhibitory activities of the compounds is determined against growth of the cells expressing a low level of microsomal dipeptidase, those expressing a high level of microsomal dipeptidase, and granulocyte progenitors that are expanded ex vivo. The human colon cancer cell line, HCT116 (American Type Culture Collection No. CCL-247), and granulocyte progenitors are used as the cells expressing only a low level of microsomal dipeptidase. A stable transfectant, HCT116/S5, into which the the human microsomal dipeptidase cDNA (herafter called MDP) connected to the CMV promoter was transfected, is used as the cells expressing a high level of microsomal dipeptidase. The dipeptidase cDNA (Satoh et al. Biotechnol. Prog. 10 (2), 134-140 (1994)) and other references) and the cloning procedures as mentioned are known in the art. HCT116, HCT116/S5, and granulocyte porogenitors are cultured in the absence and presence of the drugs. The IC₅₀ values that represent concentrations of drugs necessary to cause 50% growth inhibition as compared to cells cultured without drugs. The IC₅₀ values are determined and compared among HCT116, HCT116/S5, and granulocyte porogenitors. Although time duration of the exposure of the cells to the drugs varies depending on the cells and drugs, it can be 24 hr, 96 or 168 hr. When the cells are cultured in the presence of the drugs for 24 hr, the drugs are removed from the culture media by changing the media, and the cells are further incubated for 72 before measuring the IC50 values of the drugs.

Biological Data of Compounds in Each Example 4, 16, 17, 31, 39, 49-1, 49-2, 49-3, 49-4 and 49-11

Cytotoxicity (IC50 in nM)

[drug exposure time; 24 hr]

| Compound | HCT116 | HCT116/S5 | CFU-GM |
|---|---|---|---|
| Paclitaxel | 2.5 | 2.4 | 16 |
| Example 4 | 51 | 5.1 | 54 |
| Camptothecin | 19 | 5.6 | 6.1 |
| Example 31 | 3 | 00 | 18 170 |
| SN 38 | 3.7 | 2.2 | 1.8 |
| Example 39 | 23 | 2.8 | 20 |
| Example 49-1 | 33 | 3.3 | 61 |
| Example 49-2 | 18 | 2.6 | 31 |
| Example 49-4 | 15 | 2.1 | 54 |
| Example 49-3 | >50 | 2.9 | 250 |
| Example 49-11 | >50 | 13 | 120 |

Cytotoxicity (IC50 in nM)

[drug exposure time; 96 hr] [drug exposure time; 168 hr]

| Compound | HCT116 | HCT116/S5 | CFU-GM |
|---|---|---|---|
| DMDC | 0.2 | 0.3 | 0.07 |
| Example 16 | 1.7 | 0.23 | 2.8 |
| Example 17 | 0.99 | 0.098 | 1.1 |

HCT116: human colon cancer cell line, HCT116/S5: HCT116 transfected with the human microsomal dipeptidase cDNA, CFU-GM: human bematopoietic progenitor cells

Thus, significantly improved efficacy and safety profiles, especially in myelotoxicity, of TTCs as compared to those of the existing cytotoxics are expected in clinical situations.

A further embodiment of the present invention relates to pharmaceutical compositions containing a compound as described above. Preferably these compositions are suitable for oral or parentral administration.

As mentioned above, pharmaceutical compositions containing a compound of formula I are also an aspect of the present invention, as is a process for the manufacture of such pharmaceutical compositions, which process comprises bringing one or more compounds of formula I and, if desired, one or more other therapeutically valuable substances into a galenical administration form.

The pharmaceutical compositions may be administered orally, for example in the form of tablets, coated tablets, dragees, hard or soft gelatine capsules, solutions, emulsions or suspensions. Administration can also be carried out rectally, for example using suppositories; locally or percutaneously, for example using ointments, creams, gels or solutions; or parenterally, for example using injectable solutions.

For the manufacture of pharmaceutical preparations (tablets, coated tablets, dragées or hard gelatine capsules) these compounds can be formulated with therapeutically inert, inorganic or organic carriers. Lactose, maize starch or derivatives thereof, talc, steric acid or its salt can be used as such carriers for tablets, coated tablets, dragees and hard gelatin capsules. Suitable carriers for soft gelatin capsules are vegetable oils, waxes, fats, semi-solid or liquid polyols. Depending on the nature of the active substance no carriers are, however, generally required in the case of soft gelatin capsules. Suitable carriers for the manufacture of solutions and syrups are water, polyols, saccharose, invert sugar and glucose. Suitable carriers for injection solutions are water, alcohols, polyols, glycerine and vegetable oils. Suitable carriers for suppositories are natural or hardened oils, waxes, fats and semi-liquid polyols.

The pharmaceutical preparations can also contain preserving agents, solubilizing agents, stabilizing agents, wetting agents, emulsifying agents, sweetening agents, coloring agents, flavoring agents, salts for varying the osmotic pressure, buffers, coating agents or antioxidants. They can also contain still other therapeutically valuable substances.

The dosage can vary within wide limits and will, or course, be adjusted to the individual requirements in each particular case. In general, in the case of oral or parenteral administration to adult humans, a daily dosage of about 5 mg/m² to 500 mg/m² should be appropriate. Although the upper limit may be exceeded when this is found to be expedient.

The daily dosage can be administered as a single dose or in divided doses, or for oral or parenteral administration, it may be given as continuous infusion.

Another embodiment of the present invention is directed to the use of a anti-cancer compound as described above for the preparation of pharmaceutical compositions, preferably for the treatment of cell proliferative disorders, such as for treatment of cancer, like colorectal cancer, lung cancer, breast cancer, stomach cancer, cervical cancer and bladder cancer.

The present invention also refers to a method for treating a cell proliferative disorder, such as cancer, like a solid tumor, colorectal cancer, lung cancer, breast cancer, stomach cancer, cervical cancer and bladder cancer, comprising administering to a patient in need thereof a therapeutically effective amount of an anti-cancer compound as described above.

The following examples merely illustrate the preferred methods to identify the enzymes and/or proteins that are eligible for the activation of compounds by said enzymes and to prepare the compounds of the present invention, and are not intended to limit the scope of the invention thereto.

### EXAMPLES

### Example 1

Measurement of the Various mRNA Levels in Human Tumor and Normal Tissue by Oligonucleotide Microarrays and RT-PCR

1-1. Extraction of the RNA from Tissue

Small pieces of the 41 human colorectum tumors, 30 gastric tumors, 41 non-small cell lung cacinomas, 24 breast tumors, 15 ovarian tumor, 53 hepaticellular carcinoma, and 15 non-tumorous liver tissue (about 125 mm³ each) and 10⁷ granulocyte progenitor cells that were expanded ex vivo were rapidly frozen in liquid nitrogen. In order to extract RNA from the tissue and cells, they were suspended in TRIZOL (Life Technologies, Gaithersburg, USA, Catalog No. 15596-018) or Sepasol-RNAI (Nacalai tesque, Kyoto, Japan, Catalog No. 306-55) and homogenized twice with a Polytron (Kinematica, Littau, Switzerland) (5 sec. at maximum speed). After addition of chloroform, the tissue homogenates was centrifuged at 15,000×g for 10 min, and aqueous phases, which contained RNA, were collected. Total cellular RNA was precipitated with isopropyl alcohol, washed once with 70% ethanol and suspended in DEPC-treated water (Life Technologies, Gaithersburg, USA, Catalog No. 10813-012). After RNA was treated with 1.5 units of DNase I (Life Technologies, Gaithersburg, USA, Catalog No. 18068-015), the RNA was re-extracted with TRIZOL/chloroform, precipitated with ethanol and dissolved in DEPC-treated water. Thereafter, small molecular weight nucleotides were removed by using RNeasy Mini Kit (QIAGEN, Hilden, Germany, Catalog No.74104) according to a manufacture's instruction manual. When the purified RNA was electrophoresed on an agarose gel and stained with ethidium bromide, 28S and 18S ribosomal RNA were clearly detected, and the fluorescence of ethidium bromide bound to 28S RNA was higher than that from 18S RNA. The purified total RNA was stored at −80° C. in 70% ethanol solution until used for the cDNA synthesis.

1-2. Synthesis of cDNA and Labeled cRNA Probes

cDNA was synthesized by using reverse SuperScript Choice System (Life Technologies, Gaithersburg, USA, Catalog No. 18090-019) according to the manufacture's instruction manual. Five microgram of the purified total RNA was hybridized with an oligo-dT primer (Sawady Technology, Tokyo, Japan) that contained the sequences for the T7 promoter and 200 units of SuperScriptII reverse transcriptase and incubated at 42° C. for 1 hr. The resulting cDNA was extracted with phenol/chloroform and purified with Phase Lock Gel™ Light (Eppendorf, Hamburg, Germany, Catalog No. 0032 005.101).

cRNA was also synthesized by using MEGAscript T7 kit (Ambion, Austin, USA, Catalog No. 1334) and the cDNA as templates according to the manufacture's instruction. Approximately 5 μg of the cDNA was incubated with 2 μl of enzyme mix containing T7 polymerase, 7.5 mM each of adenosine triphosphate (ATP) and guanosine triphosphate (GTP), 5.625 mM each of cytidine triphosphate (CTP) and uridine triphosphate (UTP), 1.875 mM each of Bio-11-CTP and Bio-16-UTP (ENZO Diagnostics, Farmingdale, USA, Catalog No. 42818 and 42814, respectively) at 37° C. for 6 hr. Mononucleotides and short oligonucleotides were removed by column chromatography on CHROMA SPIN+STE-100 column (CLONTECH, Palo Alto, USA, Catalog No. K1302-2), and the cRNA in the eluates was sedimented by adding ethanol. When the 0.1 micrograms of the cRNA was separated by gel agarose gel electrophoresis and stained with etidium bromide, the length of the cRNA ranged from 300 bases to 3 kilobases. The purified cRNA was stored at below −80° C. in 70% ethanol solution until use.

1-3. Gene Expression Analysis of Tumor and Normal Tissue

Gene expression patterns of human primary tumors from live cancer patients were examined by high-density oligonucleotide microarrays (HuGeneFL array, Affymetrix, Santa Clara, USA, Catalog No. 510137) (Lipshutz, R. L. et a]. Nature Genet. 21, 20-24 (1999)). For hybridization with oligonucleotides on the chips, the cRNA was fragmented at 95° C. for 35 min in a buffer containing 40 mM Tris (Sigma, St. Louis, USA, Catalog No. T1503)-acetic acid (Wako, Osaka, Japan, Catalog No. 017-00256) (pH8.1), 100 mM potassium acetate (Wako, Osaka, Japan, Catalog No. 160-03175), and 30 mM magnesium acetate (Wako, Osaka, Japan, Catalog No. 130-00095). Hybridization was performed in 200 μl of a buffer containing 0.1M 2-(N-Morpholino) ethanesulfonic acid (MES) (Sigma, St. Louis, USA, Catalog No. M-3885), (pH6.7), 1M NaCl (Nacalai tescque, Tokyo, Japan, Catalog No. 313-20), 0.01% polyoxylene(10) octylphenyl ether (Wako, Osaka, Japan, Catalog No. 168-11805), 20 μg herring sperm DNA (Promega, Madison, USA, Catalog No. D181B), 100 μg acetylated bovine serum albumin (Sigma, St. Louis, USA, Catalog No. B-8894), 10 μg of the fragmented cRNA, and biotinylated-control oligonucleotides, biotin-5′-CTGAACGGTAGCATCTTGAC-3′ (Sawady technology, Tokyo, Japan) at 45° C. for 12 hr. After washing the chips with a buffer containing 0.01M MES (pH6.7), 0.1M NaCl, 0.001% polyoxylene(10) octylphenyl ether buffer, the chips were incubated with biotinylated anti-streptavidin antibody (Funakoshi, Tokyo, Japan, Catalog No. BA0500) and staining with streptavidin R-Phycoerythrin (Molecular Probes, Eugene, USA, Catalog No. S-866) to increase hybridization signals as described in the instruction manual (Affymetrix, Santa Clara, USA). Each pixel level was collected with laser scanner (Affymetrix, Santa Clara, USA) and levels of the expression of each cDNA and reliability (Present/Absent call) were calculated with Affymetrix GeneChip ver.3.3 and Affymetrix Microarray Suite ver.4.0 softwares. From this experiments, expression of approximately 6000 genes in the the 41 human colorectum tumors, 30 gastric tumors, 41 non-small cell lung cacinomas, 24 breast tumors, 15 ovarian tumor, 53 hepaticellular carcinoma, and 15 non-tumorous liver tissue and 10 batches of independently cultured granulocyte progenitor cells (10⁷ cells for each batch) were determined.

### Example 2

Selection of the Enzymes that are Expressed Preferably in Tumors but not in Granulocyte Progenitors and Liver

2-1. Expansion of Granulocyte Progenitors Ex Vivo

CD34-positive mononuclear cells derived from the human umbilical cord blood and bone marrow (Veritas Co, Tokyo, Japan, Catalog No.CB009F, ABM019F) were cultured on a confluent monolayer of MS5 (Itoh, K., et al. Reproducible establishment of hematopoietic supportive stromal cells from murine bone marrow. Exp. Hematol. 17, 145-153 (1989)). Mouse stromal cell lines in alpha MEM medium (Life Technologies, Gaithersburg, USA, Catalog No.12571-0063) supplemented with 10% (v/v) horse serum (HS) (Stem Cell Technologies, Vancouver, Canada, Catalog No. 06750), 10% (v/v) fetal bovine serum (FBS) (Stem Cell Technologies, Vancouver, Canada, Catalog No.06450), 50 ng/ml Flt3 ligand (PeproTec EC., London, UK., Catalog No. 300-19), 100 ng/ml SCF (PeproTech EC, London, UK., Catalog No. 300-07), and 50 ng/ml TPO (PeproTech EC, London, England, Catalog No. 300-18) at 37° C. under 5% CO₂ in humidified air. Floating hematopoietic cells were collected and stained by monoclonal antibodies against PerCP-anti-CD34 (BD pharMingen, San Diego, USA, Catalog No.340430), PE-anti-CD13 (BD pharMingen, San Diego, USA, Catalog No. 30525X) and FlTC-anti-15(BD pharMingen, SanDiego, USA, Catalog No. PM30525X). Five microlitter of each antibody was added to a 50 l of cell suspension and incubated at 4° C. for 25 min. After washing with PBS containing 10% (v/v) FCS, the expression of CD antigens were detected by using FACSCalibur (Becton Dickinson, Franklin Lakes, N.J., USA) according to the FACSCalibur Traing mannual (FACStation ver1.1. Becton Dickinson, Franklin Lakes, N.J., USA.). FACS analysis revealed that more than 90% of mononuclear cells expressed CD34 progenitor marker after they were expanded in the above condition. When these CD34-positive cells were treated with 50 ng/ml of G-CSF (Souza, L. M., et al. Recombinant human granulocyte colony-stimulating factor: effects on normal and leukemic myeloid cells. Science 232, 61-65 (1986). Pepro Tech EC, London, UK. Catalog No. 300-23.), more than 80% of the cells were differentiated into CD34-negative, CD13- and CD15-positive myeloblasts and myelocytes within 7 days, and further into neutrophils within 14 days after addition of G-CSF.

2-2. cDNAs that are Preferably Expressed in Tumors but not in Granulocyte Progenitors and other Non-Tumorous Tissue

DNA chip experiments yielded several hundreds cDNAs of which mRNA was considered to be absent (as judged by Absent-call) or expressed only at very low levels (as judged by the average difference below 50) in granulocyte progenitors and liver, but was expressed (as judged by Present-call) at certain levels (as judged by the average difference higher than 200) in tumors of breast, liver, gastric, colorectum, pancreas, or ovary in more than 50% of the patents. Among such cDNAs, more than 150 cDNAs that encode proteins possessing a known catalytic activity were selected. Those enzymes include phospholipase C, microsomal dipeptidase, arylsulfatase A, DT-diaphorase, pyrroline 5′-carboxyreductase, dehydrodiol dehydrogenase, carbonylreductase, lysyl hydroxylase, prolidase, dihydropyrimidinase, gamma-glutmyl transpeptidase, glutamine:fructose-6-phosphate amidotransferas, UDP-galactose ceramide galactosyl transferase, lysyl oxidase, enolase, glucose-6-phosphate dehydrogenase, uridine phosphorylase, stearoyl-coenzymea desaturase, epoxide hydrolase, aldolase C.

2-3. Kinetic RT-PCR Analysis

The levels of mRNA for the cDNA of TTC-activating enzyme was also verified by kinetic RT-PCR. Kinetic RT-PCR was performed by a real-time fluorescence PCR system. PCR amplification by using a LightCycler system (Roche Diagnostics, Mannheim, Germany, Catalog No. 2011468) was carried out in 20 μl of reaction mixture consisting of a master mixture containing Taq DNA polymerase, reaction buffer, dNTP mixture and SYBR Green I dye (LightCycler-DNA Master SYBR Green I, Roche Diagnostics, Mannheim, Germany, Catalog No. 2158817), 4 mM magnesium chloride (Nacalai tescque, Tokyo, Japan, Catalog No. 7791-18-6), 10 pmoles of PCR primers (Sawady Technology, Tokyo, Japan), and 2 μl of template cDNA in a LightCycler capillary (Roche Diagnostics, Mannheim, Germany, Catalog No. 1909339). The sequences of the primers to amplify the human microsomal dipeptidase cDNA were ATCGACTTGGCTCACGTGTCTGTGG, and TGTGATCCAGATGGTCGGCCACTFG. The amplification was performed within the LightCycler by 40 cycles of incubation at 95° C. for 0 sec. for denaturation, at 57-60° C. for 3-10 sec. for annealing and at 72° C. for 10 sec. for extension, with a temperature slope of 20° C./sec. Real-time PCR monitoring was achieved by measuring the fluorescent signals at the end of the annealing phase in each amplification cycle. cDNAs of normal lung, heart, liver, kidney, intsetine, colon, skin, and brain were synthesized with the RNA purchased from Strategene (Strategene, La Jolla, USA, Catalog. No. D6030-01 for brain, D6050-01 for colon, D6064-01 for heart, D6065-01 for small intestine, D6070-01 for kidney, D6080-01 for liver, D6115-01 for skin.

To qualify the integrity of isolated RNA and normalize the copy number of target sequences, kinetic RT-PCR analysis for glyceraldehyde-3-phosphate dehydrogenase (GAPDH) was also carried out by using hybridization probes. External standards for the target mRNA and GAPDH mRNA were prepared by 10-fold serial dilutions (10³ to 10⁸) of plasmid DNA. Quantification of mRNA in each sample was performed automatically by referring to the standard curve constructed at each time point according to the LightCycler software (LightCycler software version 3, Roche Diagnostics, Mannheim, Germany). The sequences of the primers to amplify GAPDH cDNA were

TCTCCAGAACATCATCCCTGCCTCTAC and
TGCTGTAGCCAAATTCGTTGTCATACC.

Although the microsomal dipeptidase mRNA was detected in kidney and small intestine, it was undetectable in lung, heart, stomach, colon, and liver. However, the levels of microsomal dipeptidase mRNA examined in 12 colorectum tumors were significantly higher than in kidney and small instestine.

Levels of the Microsomal Dipeptidase mRNA in Human Tissue

mRNA Level (as Ratio to GAPDH mRNA)

| | microsomal dipeptidase mRNA/GAPDH |
|---|---|
| Tissue | mRNA |
| Colorectal tumors | 2.6 |
| Granulocyte progenitors | 0.02 |
| Colon | 0.06 |
| Skin | <0.01 |
| Brain | <0.01 |
| Heart | <0.01 |
| Liver | 0.03 |
| Kidney | 0.58 |
| Small intestine | 0.37 |

### Example 3

13α-((2R,3S)-2-{(5S)-[5-((2S)-2-amino-4-methyl-pentanoylamino)-5-hydroxycarbonyl]-pentanoyloxy}-3-benzoylamino-3-phenylpropionyloxy)-2α-benzyloxy-4α,10β-diacetoxy-1β,7β-dihydroxy-5β,20-epoxy-tax-11-en-9-one formic acid salt

A mixture of 2α-benzyloxy-13α-((2R,3S)-3-benzoylamino-2-hydroxy-3-phenylpropionyloxy)-4α,10β-diacetoxy-1β,7β-dihydroxy-5β,20-epoxy-tax-11-en-9-one (taxol) (50.6 mg), 1-[3-(dimethylamino)propyl]-3-ethylcarbodiimide hydrochloride (13.9 mg), dimethylaminopyridine (1.0 mg), and (2S)-2-((2S)-2-benzyloxycarbonylamino-4-methyl-pentanoylamino)hexanedioic acid 1-benzylester (31.9 mg) in dichloromethane (2.0 ml) was stirred at room temperature for 22 hour. The reaction was quenched with water (3 ml), and the organic layer was separated. The aqueous layer was extracted with dichloromethane twice. The combined organic layer was washed with brine and dried over anhydrous sodium sulfate, then concentrated in vacuo. The mixture was purified by silica gel column chromatography eluted by dichloromethane-ethyl acetate (2:1) to give 13α-((2R,3S)-2-{(5S)-[5-((2S)-2-bezyloxycarbonylamino-4-methyl-pentanolyamino)-5-benzyloxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one as a pale yellow solid (74.8 mg, 97.6%).

A mixture of 13α-((2R,3S)-2-{(5S)-[5-((2S)-2-bezyloxycarbonylamino-4-methyl-pentanolyamino)-5-benzyloxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one obtained above (31.3 mg), 10% Pd/C (7.1 mg), and formic acid (0.42 ml) in methanol (6.0 ml) was stirred in the presence of H₂ at room temperature for 6.5 hour. The mixture was filtered and the filtrate was concentrated in vacuo to give 13α-((2R,3S)-2-{(5S)-[5-((2S)-2-amino-4-methyl-pentanoylamino)-5-hydroxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one formic acid salt salt as a pale yellow solid (23.6 mg, 91%).

¹H-NMR (CDCl₃): δ0.8˜0.9(6H,m),0.98(3H,s), 1.20(3H,s),1.48(3H,s),1.2˜1.6(2H,m),1.55˜1.8(7H,m),1.75(3H,s),2.16(3H,s)2.3˜2.5(6H, m),2.25(3H,s),3.50(1H,br),3.80(1H,m),3.95(2H,m),4.08(1H,m),4.7˜4.9(3H,m)5.32(1H, d,J=11 Hz),5.39(1H,d,J=8 Hz),5.50(1H,t,J=9 Hz),5.78(1H,drt,J=8.8 Hz),6.29(1H,s)7.17(1 H, m),7.4˜7.8(11H,m),7.87(2H,m),7.98(2H,m),9.28(1H,d,J=11 Hz); ESI-MS: m/z 1110 (M⁺-HCO₂H)

The following compounds in examples 4 and 5 were prepared from (2S)-2-((2S)-2-benzyloxycarbonylamino-3-phenyl-propionylamino)hexanedioic acid 1-benzylester or (2S)-2-((2S)-2-benzyloxycarbonylamino-propionylamino)hexanedioic acid 1-benzylester in a similar manner to Example 3.

### Example 4

13α-((2R,3S)-2-{(5S)-[5-((2S)-2-amino-propinoylamino)-5-hydroxycarbonyl]pentanoylox}-3-benzoylamino-3-phenylpropionyloxy)-2α-benzyloxy-4α,10β-diacetoxy-1β,7β-dihydroxy-5β,20-epoxy-tax-11-en-9-one formic acid salt

¹H-NMR (CDCl₃): δ1.04(3H,s),1.10(3H,s),1.20(3H,d,J=11 Hz),1.49(3H,s),1.5˜1.8(6H,m),1.75(3H,s),2.14(3H,s),2.3˜2.5(6H,m),2.25(3 H,s), 3.50(1H,br),3.80(1H,m),3.98(2H,m),4.12(1H,m),4.7˜4.9(3H,m),5.32(1H,d,J=11 Hz),5,42 (1H,d,J=8 Hz),5.52(1H,t,J=9 Hz),5.80(1H,drt,J=8.8 Hz),6.39(1H,s),7.18(1H,m),7.4˜7.8(11H, m),7.87(2H,m), 7.98(2H,m), 9.31(1H,d,J=11 Hz); ESI-MS: m/z1068(M⁺-HCO₂H).

### Example 5

13α-((2R,3S)-2-{(5S)-[5-((2S)-2-amino-3-phenyl-propinoylamino)-5-hydroxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one formic acid salt

¹H-NMR (CDCl₃): δ0.98(3H,s),1.00(3H,s),1.47(3H,s), 1.4˜1.8(6H,m),1.75(3H,s),2.08(3H,s),2.3˜2.5(8H,m),2.20(3H,s),3.60(1H,br),3.80(1H,m), 3.98(2H,m),4.10(1H,m),4.70(1H,br)4.9(2H,br),5.31(1H,d,J=11 Hz),5.40(1H,d,J=8 Hz), 5.53(1H,t,J=9 Hz),5.79(1H,drt,J=8.8 Hz),6.29(1H,s),7.15˜7.30(6H,m),7.4˜7.8(11H,m),7.87 (2H,m),7.98(2H,m), 9.31(1H,d,J=11 Hz); ESI-MS: m/z 1144 (M⁺-HCO₂H).

### Example 6

(2R)-((2S)-amino-3-cyclohexyl-propionylamino)-(3S)-[1-((4S)-hydroxy-(5R)-hydroxymethyl-3-methylene-tetrahydro-furan-(2R)-yl)-2-oxo-1,2-dihydro-pyrimidine-4-ylcarbamoyloxy]-butyric acid

a) To a stirred solution of 2.5 g (8.09 mmol) of BOC-D-Thr(Bzl)-OH in 200 mL of CH₂Cl₂ (dehydrated) was added 1.3 mL (8.9 mmol) of 2-(trimethylsilyl)ethanol, 0.5 g (4.04 mmol) of DMAP and 2.3 g (12.13 mmol) of WSC HCl. The mixture was stirred for 5 hrs under Ar at room temperature. The reaction was quenched by addition of water, and organic layer was separated. The aqueous layer was extracted with EtOAc. The combined organic layer was washed with water and brine. The extract was dried over anhydrous Na₂SO₄ and filtered.

The solvent was removed under reduced pressure. The crude product was purified by flash chromatography on SiO₂ (eluent: 20% EtOAc/Hexane) to give (3S)-benzyloxy-(2R)-tert-butoxycarbonylamino-butyric acid 2-trimethysilanyl-ethyl ester as a colorless viscous

¹H-NMR: (270 MHz, CDCl₃) δ0.02(9H, s), 0.90(2H, d.d.d, J=6.6, 3.3, 2.6 Hz), 1.23(3H, d, J=6.3 Hz), 1.43(9H, s), 4.03-4.26(4H, m), 4.34(1H, d, J=12.0 Hz AB), 4.54(1H, d, J=12.0 Hz AB), 5.26(1H, d, J=9.6 Hz), 7.18-7.33(5H, m); MS: (LCMS) m/z 410 [M+H]⁺, 432[M+Na]⁺.

b) To a stirred solution of 2.68 g (6.55 mmol) of (3S)-benzyloxy-(2R)-tert-butoxycarbonylamino-butyric acid 2-trimethysilanyl-ethyl ester in 50 mL of CH₂Cl₂ (dehydrated) was added 4.5 ml of TFA at room temperature. The mixture was stirred for 7 hrs and the mixture was concentrated under reduced pressure to afford (2R)-amino-(3S)-benzyloxy-butyric acid 2-trimethysilanyl-ethyl ester trifluoro-acetic acid salt as a pale yellow viscous oil (3.555 g quant.). This product was used in the following reaction step without further purification.

¹H-NMR: (270 MHz, CDCl₃) δ0.03(9H, s), 0.88(2H, d.d.d, J=11.9, 5.9, 5.3 Hz), 1.36(3H, d, J=6.3 Hz), 3.94(1H, d, J=3.3 Hz), 4.11(2H, m), 4.23(1H, d.d, J=10.2, 6.9 Hz) 4.37(1H, d, J=11.9 Hz AB), 4.61(1H, d, J=11.9 Hz AB), 5.26(1H, d, J=9.6 Hz), 7.18-7.34(5H, m); MS: (LCMS) m/z 310 [M+H]⁺.

c) To a stirred solution of 181.6 mg (0.439 mmol) of (2R)-amino-(3S)-benzyloxy-butyric acid 2-trimethysilanyl-ethyl ester trifluoro-acetic acid salt in 5 mL of CH₂Cl₂(dehydrated) was added 131 mg (0.484 mmol) of N-BOC-(L)-cyclohexylalanine and 170 mg(0.878 mmol) of WSC+HCl at room temperature.

The mixture was stirred for 18 hrs under Ar at room temperature. The reaction was quenched by addition of water, and organic layer was separated. The aqueous layer was extracted with EtOAc. The combined organic layer was washed with water and brine. The extract was dried over anhydrous Na₂SO₄ and filtered. The solvent was removed under reduced pressure. The crude product was purified by flash chromatography on SiO₂ (eluent: 10% EtOAc/hexane) to give (3S)-benzyloxy-(2R)-((2S)-tert-butoxycarbonylamino-3-cyclohexyl-propionylamino)-butyric acid 2-trimethysilanyl-ethyl ester as a colorless viscous oil (63.1 mg, 26%).

¹H-NMR: (270 MHz, CDCl₃) δ0.02(9H, s), 0.91(2H, d.d.d,J=10.2, 7.5, 6.9 Hz), 0.24-1.82(13H, m), 1.21(3H, d, J=6.3 Hz), 1.43(9H, s), 4.03-4.26(3H, m), 4.37(1H, d, J=11.9 Hz AB), 4.57(1H, d, J=11.9 Hz AB), 4.59(1H, d.d, J=9.6, 2.3 Hz), 4.83(1H, m), 6.77(1H, d, J=8.9 Hz), 7.21-7.64(5H, m); MS: (LCMS) m/z 563 [M+H]⁺.

d) To a stirred solution of 61.3 mg (0.109 mmol) of (3S)-benzyloxy-(2R)-((2S)-tert-butoxycarbonylamino-3-cyclohexyl-propionylamino)-butyric acid 2-trimethysilanyl-ethyl ester in 10 mL of CH₂Cl₂ (dehydrated) was added 1.0 mL of TFA at room temperature. The mixture was stirred for 1 hr and then concentrated under reduced pressure to give (2R)-((2S)-amino-3-cyclohexyl-propionylamino)-3-benzyloxy-butyric acid 2-trimethysilanyl-ethyl ester trifluoro-acetic acid salt as a colorless viscous oil (77.9 mg, quant.). This product was used in the next reaction step without further purification.

¹H-NMR (270 MHz, CDCl₃): δ0.02(9H, s), 0.88(2H, m), 0.8-1.74(13H, m), 1.19(3H, d, J=6.3 Hz), 4.00-4.21(3H, m), 4.11(2H, m), 4.35(1H, d, J=11.9 Hz AB), 4.54(1H, d.d, J=8.2, 2.3 Hz), 4.57(1H, d, J=11.9 Hz AB), 7.20-7.35(5H, m); MS: (LCMS) m/z 463 [M+H]⁺.

e) To a stirred solution of 75.5 mg (0.131 mmol) of (2R)-((2S)-amino-3-cyclobexyl-propionylamino)-3-benzyloxy-butyric acid 2-trimethysilanyl-ethyl ester trifluoro-acetic acid salt in 5.0 mL of THF was added dropwise 130 mL of 1 mol/l NaOH at room temperature. The pH of the reaction mixture was adjusted to pH 7. Then, 74 mg (0.262 mmol) of 2-(trimethylsilyl)ethyl p-nitrophenylcarbonate was added to the reaction mixture and the mixture was warmed up to 60° C. in an oil bath. After stirring for 1 day at 60° C., the mixture was cooled to room temperature and the mixture was diluted with EtOAc (20 mL) and water (20 mL), and organic layer was separated. The aqueous layer was extracted with EtOAc. The combined organic layer was washed with water and brine. The extract was dried over anhydrous Na₂SO₄ and filtered. The solvent was removed under reduced pressure. The crude product was purified by flashchromatography on SiO₂ (eluent: 10% to 15% EtOAc/hexane) to give (3S)-benzyloxy-(2R)-[3-cyclohexyl-(2S)-(2-trimethylsilanyl-ethoxycarbonylanino)-propionylamino]-butyric acid 2-trimethysilanyl-ethyl ester as a colorless viscous oil (49.2 mg, 62%).

¹H-NMR: (270 MHz, CDCl₃) δ0.02(18H, s), 0.87-1.04(6H, m), 1.10-1.28(2H, m), 1.21(3H, d, J=6.3 Hz), 1.37-1.57(3H, m), 1.67-1.83(6H, m), 4.03-4.23(4H, m) 4.36(1H, m), 4.38(1H, d, J=11.9 Hz AB), 4.58(1H, d, J=11.9 Hz AB), 4.59(1H, d.d, J=9.2, 2.3 Hz), 4.97(1H, m), 6.68(1H, d, J=9.2 Hz), 7.23-7.36(5H, m); MS: (LCMS) m/z 607 [M+H]⁺, 629[M+Na]⁺.

f) To a solution of 38.9 mg (0.064 mmol) of (3S)-benzyloxy-(2R)-[3-cyclohexyl-(2S)-(2-trimethylsilanyl-ethoxycarbonylanino)-propionylamino]-butyric acid 2-trimethysilanyl-ethyl ester in 10 mL of EtOAc was added 10% Pd/C. The reaction mixture was stirred vigorously under H₂ atmosphere. After stirring for 2 hrs, the mixture was filtered through a short pad Celite column. The filtrate was concentrated under reduced pressure to afford (2R)-[3-cyclohexyl-(2S)-(2-trimethylsilanyl-ethoxycarbonylanino)-propionylamino]-(3S)-hydroxy-butyric acid 2-trimethysilanyl-ethyl ester as a colorless viscous oil. The product was used in the next reaction step without further purification

¹H-NMR (270 MHz, CDCl₃): δ0.04(9H, s), 0.05(9H, s), 0.09-1.07(6H, m), 1.11-1.30(2H, m), 1.22(3H, d, J=6.3 Hz), 1.37-1.56(3H, m), 1.67-1.82(6H, m), 2.09(1H, d, J=5.3 Hz), 4.15-4.36(7H, m), 4.54(1H, d.d, J=8.9, 2.6 Hz), 4.95(1H, d, J=7.6 Hz), 6.76(1H, d, J=8.6 Hz); MS: (LCMS) m/z 517 [M+H]⁺, 539[M+Na]⁺.

g) To a stirred solution of 39.1 mg (0.075 mmol) of (2R)-[3-cyclohexyl-(2S)-(2-trimethylsilanyl-ethoxycarbonylanino)-propionylamino]-(3S)-hydroxy-butyric acid 2-trimethysilanyl-ethyl ester in 5.0 mL of CH₂Cl₂ (dehydrated) was added 30 mg (0.151 mmol) of 4-nitrophenyl chloroforinate and 1.0 mL of pyridine at room temperature. After stirring for 3 hrs, The reaction was quenched by addition of water, and organic layer was separated. The aqueous layer was extracted with EtOAc. The combined organic layer was washed with water and brine. The extract was dried over anhydrous Na₂SO₄ and filtered. The solvent was removed under reduced pressure. The crude product was purified by flash chromatography on SiO₂ (eluent: 20% EtOAc/hexane) to give (2R)-[3-cyclohexyl-(2S)-(2-trimethylsilanyl-ethoxycarbonylanino)-propionylamino]-(3S)-(4-nitro-phenoxycarbonyloxy)-butyric acid 2-trimethysilanyl-ethyl ester as a colorless solid (64.7 mg).

¹H-NMR (270 MHz, CDCl₃): δ0.02(9H, s), 0.04(9H, s), 0.97-1.04(6H, m), 1.17-1.38(3H, m), 1.41(3H, d, J=6.3 Hz), 1.47-1.83(8H, m),4.16-4.33(5H, m), 4.84(1H, d.d, J=9.2, 2.6 Hz), 4.90(1H, m), 7.38(2H, d, J=9.2 Hz), 8.2(2H, d, J=9.2 Hz); MS: (LCMS) m/z 681 [M+H]⁺.

h) To a stirred solution of 62.2 mg (0.09 mmol) of (2R)-[3-cyclohexyl-(2S)-(2-trimethylsilanyl-ethoxycarbonylanino)-propionylamino]-(3S)-(4-nitro-phenoxycarbonyloxy)-butyric acid 2-trimethysilanyl-ethyl ester in 5mL of THF(dehydrated) was added 85 mg (0.182 mmol) of 3′,5′-di-tert-butyldimethylsilyl-DMDC at room temperature. The reaction mixture was warm up to 60° C. in an oil bath. After stirring for 4 days, the mixture was cooled to room temperature and the mixture was concentated under reduced pressure. The oily residue was dissolved in EtOAc and washed with sat. NaHCO₃, water and brine. The organic layer was dried over anhydrous Na₂SO₄ and filtered. The solvent was removed under reduced pressure. The crude product was purified by flash chromatography on SiO₂ (eluent: 20% to 30% EtOAc/hexane) to give (2R)-[3-cyclohexyl-(2S)-(2-trimethylsilanyl-ethoxycarbonylanino)-propionylamino]-(3S)-{1-[(4S)-(tert-butyldimethylsilanyloxy)-(5R)-(tert-butyldimethylsilanyloxymethyl)-3-methylene-tetrahydro-furan-(2R)-yl]-2-oxo-1,2-dihydro-pyrimidine-4-ylcarbamoyloxy}-butyric acid 2-trimethysilanyl-ethyl ester as a colorless solid (38.8 mg, 50% 2 steps).

¹H-NMR (270 MHz, CDCl₃): δ0.00(12H, s), 0.01(9H, s), 0.02(9H, s), 0.92(9H, s), 0.95(9H, s), 0.86-1.81(13H, m), 1.33(3H, d, J=6.3 Hz), 3.82(2H, m),4.17(4H, m), 4.40(1H, br.s), 4.78(2H, m), 5.32(3H, m), 5.66(1H, br.s), 6.79(1H, br.s), 7.1(1H, br.d, J=6.9 Hz), 7.65(1H, br.s), 8.16(1H, br.d, J=6.9 Hz), 9.80(1H, br.s); MS: (LCMS) m/z 1010 [M+H]⁺.

i) To a stirred solution of 37.3 mg (0.037 mmol) of (2R)-[3-cyclohexyl-(2S)-(2-trimethylsilanyl-ethoxycarbonylanino)-propionylamino]-(3S)-{1-[(4S)-(tert-butyldimethylsilanyloxy)-(5R)-(tert-butyldimethylsilanyloxymethyl)-3-methylene-tetrahydro-furan-(2R)-yl]-2-oxo-1,2-dihydro-pyrimidine-4-ylcarbamoyloxy}-butyric acid 2-trimethysilanyl-ethyl ester in 5.0 mL of THF (dehydrated) was added 220 mL of n-tetrabutylammonium fluoride (1 mol/L in THF) at room temperature.

After stirring for 1 hr, The solvent was removed under reduced pressure, the yellowish oily residue was purified by preparative HPLC(C18)* to give (2R)-((2S)-amino-3-cyclohexyl-propionylamino)-(3S)-[1-((4S)-hydroxy-(5R)-hydroxymethyl-3-methylene-tetrahydro-furan-(2R)-yl)-2-oxo-1,2-dihydro-pyrimidine-4-ylcarbamoyloxy]-butyric acid as a colorless solid (12.1 mg, 61%).

*HPLC condition: column; 2×25 cm (TSK-gel 80-TS ODS), eluent; 5% MeCN/H₂O to 100% MeCN (30 min. liner gradient), flow rate; 9 mL/min., detection; photodiode array.

¹H-NMR (270 MHz, CD₃OD): δ0.95-1.82(13H, m), 2.65(3H, d, J=6.6 Hz), 3.79(2H, m) 3.95(2H, m), 4.46(1H, d, J=4.3 Hz), 4.68(1H, m), 5.42(1H, d.d, J=6.6, 4.3 Hz), 5.47(2H, t, J=2.0 Hz), 6.67(1H, d, J=1.3 Hz), 7.26(1H, d, J=7.6 Hz), 8.20(1H, d, J=37.6 Hz); MS: (FABMS) m/z 538 [M+H]⁺.

The following compounds in examples 7-13 were prepared from DMDC using different dipeptide (threonine) derivatives of formula (VIII) by the method similar to Example 6.

### Example 7

(2R)-((2S)-Amino-4-methyl-pentanoylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid was prepared from (3S)-hydroxy-(2R)-[4-methyl-(2S)-(2-trimethylsilanyl-ethoxycarbonylamino)-pentanoylamino]-butyric acid 2-trimethylsilanyl-ethyl ester

¹H-NMR: (270 MHz, CD₃OD) δ0.98(6H, d,J=4.9 Hz), 1.31(3H, d,J=6.3Hz), 1.63-1.76(3H, m), 3.77-3.99 (4H, m), 4.46(1H, d, J=4.0 Hz), 4.73(1H, m), 5.41(1H, m) 5.46(2H, s), 6.66(1H, s), 7.26(1H, d, J=7.3 Hz), 8.19(1H, d, J=7.3 Hz); MS: (FABMS) m/z 498 [M+H]⁺.

### Example 8

(2R)-((2S)-Amino-3-biphenyl-4-yl-propionylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid was prepared from (2R)-[3-biphenyl-4-yl-(2S)-(2-trimethylsilanyl-ethoxycarbonylamino)-propionylamino]-(3S)-hydroxy-butyric acid 2-trimethylsilanyl-ethyl ester

¹H-NMR: (270 MHz, CD₃OD) δ0.92(3H, d, J=6.6 Hz), 3.20 (2H, m), 3.76(2H,m), 3.84(1H, m), 4.26(1H, t, J=7.56 Hz), 4.35(1H, d, J=3.3 Hz), 4.68(1H, m), 5.28(1H, d.d, J=6.3, 3.0 Hz), 5.47(2H, m), 6.54(1H, d, J=1.6 Hz), 7.11(1H, d, J=7.6 Hz), 7.23-7.60(9H,m), 8.13(1H, d, J=7.6 Hz); MS:(FABMS) m/z 608 [M+H]⁺.

### Example 9

2(R)-[2(S)-Amino-3-biphenyl-4-yl-propionylamino]-3-{1-[4(S)-hydroxy-5(R)-hydroxymethyl-3-methylene-tetrahydro-furan-2(R)-yl]-2-oxo-1,2-dihydro-pyrimidin-4-ylcarbamoyloxy}-propionic acid was prepared from 2(R)-[3-Biphenyl-4-yl-2(S)-(9H-fluoren-9-ylmethoxycarbonylamino)-propionylamino]-3-hydroxy-propionic acid 2-trimethylsilanyl-ethyl ester

¹H-NMR: (270 MHz, CD₃OD) δ3.13 (2H, ddd,J=13.2, 8.9, 8.2 Hz), 3.76(1H,m), 3.87(2H, m), 4.13(1H, dd,J=8.2, 6.9 Hz), 4.21(1H, dd, J=10.9, 3.3 Hz), 4.35(1H, dd, J=10.9, 5.3 Hz), 4.59(1H, dd, J=5.2, 3.0 Hz), 4.70(1H, m), 5.48(2H, d, J=2.3 Hz), 6.62(1H,d, J=1.3 Hz), 6.98(1H, d, J=7.6 Hz), 7.20-7.57(9H, m), 8.04(1H, d, J=7.6 Hz); MS:(FABMS) m/z 594 [M+H]⁺.

### Example 10

(2R)-((2S)-Amino-3-naphthalen-2-yl-propionylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid was prepared from (3S)-hydroxy-(2R)-[3-naphthalen-2-yl-(2S)-(2-trimethylsilanyl-ethoxycarbonylamino)-propionylamino]-butyric acid 2-trimethylsilanyl-ethyl ester

¹H-NMR: (270 MHz, CD₃OD) δ0.69(3H, d, J=6.6 Hz), 3.20 (2H, m), 3.80(1H, m), 3.90(2H, m), 4.29-4.35(2H, m), 4.67(1H, m), 5.24(1H, d.d, J=6.6, 3.0 Hz), 5.46(2H, s), 6.65(1H, d, J=1.7 Hz), 7.12(1H, d, J=7.5 Hz), 7.38-7.47(3H, m), 7.81-7.87(4H, m), 8.13(1H, d, J=7.5 Hz); MS: (FABMS) m/z 582 [M+H]⁺.

### Example 11

(2R)-{(2S)-Amino-3-[4-(4-hydroxy-phenoxy)-phenyl]-propionylamino}-3-[1-((4S)-hydroxy-(5R)-hydroxymethyl-3-methylene-tetrahydro-furan-2-yl)-2-oxo-1,2-dihydro-pyrimidin-4-ylcarbamoyloxy]-butyric acid was prepared from (3S)-Hydroxy-(2R)-[3-{4-[4-(tert-butyl-dimethyl-silanyloxy)-phenoxy]-phenyl}-(2S)-(2-trimethylsilanyl-ethoxycarbonylamino)-propionylamino]-butyric acid 2-trimethylsilanyl-ethyl ester

¹H-NMR (CD₃OD): δ0.95(3H, d, J=6.2 Hz), 2.89-3.08(2H, m), 3.74-3.98(3H, m), 4.34(1H, d, J=2.9 Hz), 4.66(1H, m), 5.30(1H, m), 5.44(2H, s), 6.65(1H, s), 6.72(2H, d, J=7.1 Hz), 6.80(2H, d, J=7.0), 6.82(2H, d, J=8.51 Hz), 7.18(2H, d, J=8.6 Hz), 7.22(1H, d, J=7.6 Hz), 8.13(1H, d, J=7.6 Hz); MS: LC-MS m/z 640.0[M+H]⁺.

### Example 12

(2R)-[(2S)-amino-3-(4-methoxy-phenyl)-propionylamino]-(35)-[1-[(4S)-hydroxy-(5R)-hydroxymethyl-3-methylene-tetrahydro-furan-2-yl]-2-oxo-1,2-dihydro-pyrimidin-4-ylcarbamoyloxy]-butyric acid was prepared from (3S)-hydroxy-(2R)-[3-(4-methoxy-phenyl)-(2S)-(2-trimethylsilanyl-ethoxycarbonylamino)-propionylamino]-butyric acid 2-trimethylsilanyl-ethyl ester

¹H-NMR (CD₃OD) δ0.93(3H, d,J=6.6 Hz), 2.91-3.14(2H, m), 3.72(s, 3H), 3.73-3.95(3H, m), 4.11(1H, t, J=6.7 Hz), 4.34(1H, br), 4.65(1H, m), 5.31(1H, m), 5.45(2H, s), 6.65(1H, s), 6.86(2H, d, J=6.9 Hz), 7.17(2H, d, J=7.0), 7.21(1H, d, 8.6 Hz), 8.15(1H, d, J=6.9 Hz); ESI-MS ml/z 561.9[M+H]⁺, 434, 297, 150.

### Example 13

(2R)-[(2S)-Amino-4-ethylsulfanyl-butyrylamino]-(3S)-[1-[(4S)-hydroxy-(5R)-hydroxymethyl-3-methylene-tetrahydro-furan-(2R)-yl]-2-oxo-1,2-dihydro-pyrimidin-4-ylcarbamoyl]-butyric acid was prepared from (2R)-[4-ethylsulfanyl-(2S)-(3-trimethylsilanyl-propionylamino)-butylamino]-(3S)-hydroxy-butylic acid 2-trimethylsilanyl-ethyl ester

H-NMR: (400 MHz, CD₃OD) δ1.22(3H,t,J=7.6 Hz), 1.34(3H,d,J=6.4 Hz), 2.05(1H,m), 2.14(1H,m), 2.52-2.62(4H,m), 3.80(2H,m), 3.93(1H,m), 4.08(1H,t,J=6.6 Hz 4.49(1H,d,J=4.0 Hz), 4.68(1H,m), 5.42(1H,dd,J=6.4,4.0 Hz), 5.47(2H,br), 6.67(1H,s), 7.25(1H,d,J=7.2 Hz), 8.20(1H,d,J=7.6 Hz); MS: (FAB-MS) m/z 530[M+H]⁺.

### Example 14

(2R)-[(2S)-Amino-3-(1H-indol-3-yl)propionylamino]-4-[1-((4S)-hydroxy-(5R)-hydroxymethyl-3-methylenetetrahydrofuran-2-yl)-2-oxo-1,2-dihydropyrimidin-4-ylcarbamoyl]butyric acid

(a) A mixture of Teoc-L-Trp-OH (25 g), (2R)-aminopentanedioic acid 5-benzyl ester-1-(2-trimethylsilanylethyl)ester hydrochloride (23 g) prepared according to a literature procedure (Pacofsky, Gregory J ; J. Med. Chem, 41, 11, 1998, 1894-1908), WSCI (14 g) and diispropylethylamine (25 ml) in dichloromethane (250 ml) was stirred at room temperature under Ar gas atmosphere for 22 hour. The reaction mixture was quenched with water and the organic layer was separated. The aqueous layer was extracted with dichloromethane. The combined organic layer was washed with brine and dried over anhydrous sodium sulfate then concentrated in vacuo.

The crude residue was purified by silica gel column chromatography eluted by n-hexane-ethyl acetate(2:1) to give (2R)-[3-(1H-indol-3-yl)-2S-(2-trimethylsilanylethoxy-carbonylamino)propionylamino]pentanedioic acid 5-benzyl ester 1-(2-trimethylsilanyl-ethyl)ester as a colorless oil (39 g, 93.2%).

¹H-NMR:[270 MHz: CDCl₃]: δ0.023-0.005 (18H, m), 0.88-0.98 (4H, m), 0.6-2.0 (4H, m), 3.07-3.15 (1H,m), 3.25-3.30 (1H, m), 4.06-4.17 (4H, m), 4.4-4.6 (2H, m), 5.08 (2H,), 5.2-5.3 (1H, brs), 6.18 (1H, d, J=7.6 Hz), 6.96 (1H, s), 7.0-7.25 (3H, m ), 7.3-7.4 (5H, m ), 7.66 (1H, d, J=7.3 Hz ), 7.81 (1H, brs); FAB-MS: m/z 668 [[M+H]⁺.

(b) A mixture of (2R )-[3-(1H-indol-3-yl)-2S -(2-trimethylsilanylethoxy-carbonylamino)propionylamino]pentanedioic acid 5-benzyl ester 1-(2-trimethylsilanylethyl)ester (36 g) and 10% Pd-C (3.6 g) in ethyl acetate (350 ml) was stirred in the presence of H₂ gas at room temperature for 22 hour.

The reaction mixture was filtered and the filtrate was evaporated in vacuo to give (2R)-[3-(1H-indol-3-yl)-2S -(2-trimethylsilanylethoxycarbonylamino)propionylamino]-pentanedioic acid 1-(2-trimethylsilanylethyl)ester as a colorless oil (32 g,).

¹H-NMR:[270 MHz: CDCl₃]: δ0.018-0.01 (18H, m), 0.85-1.0 (4H, m), 0.6-2.1 (4H, m), 3.1-3.4 (2H, m), 4.0-4.2 (4H, m), 4.4-4.6 (2H, m), 5.3-5.4 (1H, brs), 6.5-6.6 (1H, brs), 7.0-7.2 (3H, m), 7.33 (1H, d, J=7.6 Hz), 7.61 (1H, d, J=8.2 Hz), 8.33(1H, brs); LC-MS: m/z 578 [M+H]⁺.

(c) A mixture of (2R)-[3-(1H-indol-3-yl)-2S -(2-trimethylsilanylethoxy-carbonylamino) propionylamino]pentanedioic acid 1-(2-trimethylsilanylethyl)ester (29 g), 3′,5′-bis-O-(tert-butyldimethylsilyl)-2′-deoxy-2′-methylidenecytidine (24 g), BOP reagent (27 g) and diispropylethylamine (12 ml) in dichloromethane (500 ml) was stirred at room temperature under Ar gas atmosphere for 19 hour. The reaction mixture was quenched with water and the organic layer was separated. The aqueous layer was extracted with dichloromethane. The combined organic layer was washed with brine and dried over anhydrous sodium sulfate then concentrated in vacuo.

The crude residue was purified by silica gel column chromatography eluted by n-hexane-acetone (3:1) to give 4-[1-(4S-tert-butyldimethylsilanyloxy-5R-tert-butyldimethylsilanyloxymethyl-3-methylenetetrahydrofuran-2-yl)-2-oxo-1,2-dihydropyrimidin-4-ylcarbamoyl]-2R-[3-(1H-indol-3-yl)-2S-(2-trimethylsilanylethoxycarbonylamino)propionylamino]butyric acid 1-(2-trimethylsilanylethyl)ester as a colorless amorphous solid (45 g, 86.4%).

¹H-NMR:[270 MHz: CDCl₃]: δ0.01-0.13 (30H, m), 0.8-1.0 (22H, m), 1.6-2.1 (4H, m), 3.1-3.3(2H, m), 3.78-3.85 (2H, m), 4.0-4.2 (5H, m), 4.35-4.45 (1H, m) 4.45-4.65 (1H, m), 4.77-4.79 (1H, m), 5.33-5.34 (1H, m), 5.44 (1H, d, J=7.6 Hz), 5.6-5.7 (1H, m), 6.51 (1H, d, J=7.9 Hz), 6.78 (1H, d, J=1.3 Hz), 7.07-7.23 (4H, m), 7.35-7.38 (1H, m), 7.64 (1H, d, J=7.3 Hz), 8.17 (1H, d, J=7.6 Hz ), 8.63 (1H, brs), 8.86 (1H, brs); FAB-MS: m/z 1027 [M+H]⁺.

(d) A mixture of 4-[1-((4S)-tert-butyldimethylsilanyloxy-(5R)-tert-butyldimethylsilanyloxymethyl-3-methylenetetrahydrofuran-2-yl)-2-oxo-1,2-dihydropyrimidin-4-ylcarbamoyl]-(2R)-[3-(1H-indol-3-yl)-(2S)-(2-trimethylsilanylethoxycarbonylamino)propionylamino]butyric acid 1-(2-trimethylsilanylethyl)ester (2 g) and TBAF [1 mol/l in THF] (39 ml) in tetrahydrofuran (20 ml) was stirred at room temperature under Ar gas atmosphere for 23 hour. The reaction mixture was evaporated in vacuo. The crude residue was purified by ion-exchange chromatography [Amberlite® CG-50] eluted by methanol and then preparative reverse phase HPLC eluted by H₂O-acetonitrile (85:15) to give (2R)-[(2S)-Amino-3-(1H-indol-3-yl)propionylamino]-4-[1β-((4S)-hydroxy-(5R)-hydroxymethyl-3-methylenetetrahydrofuran-2-yl)-2-oxo-1,2-dihydropyrimidin-4-ylcarbamoyl]butyric acid as a white solid (449 mg, 41.6%).

1H-NMR:[270 MHz: CD3OD]: δ1.6-2.0 (4H, m), 3.17 (1H, dd, J=7.3, 14.2 Hz), 3.2-3.4 (1H, m), 3.76-3.83 (2H, m), 3.93 (1H, dd, J=3.3, 13.2 Hz), 4.06-4.17 (2H, m), 4.66-4.69 (1H, m), 5.44-5.47 (2H, m), 6.67 (1H, d, J=1.7 Hz), 6.98-7.08 (2H, m), 7.17 (1H, s), 7.29-7.32 (2H, m), 7.58-7.61 (1H, m), 8.16 (1H, d, J=7.6 Hz); FAB-MS: m/z 555 [M+H]⁺.

The following compounds in examples 15-18 were prepared from DMDC using a different dipeptide (glutamic acid) derivative of formula (VII) by the method similar to Example 14.

### Example 15

(2R)-((2S)-Amino-3-cyclohexylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid was prepared from (2R)-[3-cyclohexyl-(2S)-(2-trimethylsilanylethoxycarbonylamino) propionylamino]pentanedioic acid 1-(2-trimethylsilanylethyl)ester

¹H-NMR:[270 MHz: DMSO-d6]: δ0.7-1.0 (2H, m), 1.0-1.8 (11H, m), 1.8-2.0 (2H, m), 2.3-2.5 (2H, m), 3.5-3.8 (4H, m), 4.0 (1H, m), 4.51-4.53 (1H, m), 5.31 (1H, s), 5.34 (1H, s), 6.55 (1H, s), 7.20 (1H, d, J=7.6 Hz), 8.10 (1H, d, J=7.3 Hz ; FAB-MS: m/z 522 [M+H]⁺.

### Example 16

(2R)-((2S)-Amino-3-biphenyl-4-ylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid was prepared from (2R)-[3-biphenyl-4-yl-(2S)-(2-trimethylsilanylethoxycarbonylamino) propionylamino]pentanedioic acid 1-(2-trimethylsilanylethyl)ester

¹H-NMR:[500 MHz: DMSO-d6]: δ1.7-1.8 (1H, m), 1.9-2.0 (1H, m), 2.2-2.4 (2H, m), 2.76-2.80 (1H, m), 3.0-3.04 (1H, m), 3.5-4.0 (4H, m), 4.08 (1H, m), 4.52-4.54 (1H, m), 5.12 (1H, brs), 5.32 (1H, s), 5.35 (1H, s), 5.74 (1H, brs), 6.55 (1H, s), 7.17 (1H, d, J=8.0 Hz), 7.28-7.40 (5H, m), 7.55-7.60 (4H, m), 8.09 (1H, d, J=7.5 Hz), 8.11 (1H, brs), 11.0 (1H, brs); FAB-MS: m/z 592 [M+H]⁺.

### Example 17

(2R)-((2S)-Amino-3-naphthalen-2-ylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid was prepared from (2R)-[3-naphthalen-2-yl-(2S)-(2-trimethylsilanylethoxycarbonylamino)propionylamino]pentanedioic acid 1-(2-trimethylsilanylethyl)ester

¹H-NMR:[270 MHz: DMSO-d6]: δ1.7-2.0 (2H, m), 2.3-2.35 (2H, m), 2.89 (1H, dd, J=8.6, 13.5 Hz), 3.19 (1H, dd, J=5.3, 13.5 Hz), 3.5-4.0 (4H, m), 4.0-4.1 (1H, m), 4.51-4.54 (1H, m), 5.31 (1H, s), 5.34 (1H, s), 6.55 (1H, s), 7.19 (1H, d, J=7.6 Hz), 7.39-7.47 (3H, m), 7.73 (1H, s), 7.80-7.84 (3H, m), 8.11 (1H, d, J=7.6 Hz), 8.20 (1H, brs); FAB-MS: m/z 566 [M+H]⁺.

### Example 18

(2R)-((2S)-Amino-3-cyclohexyl-propionylamino)-(3S)-[1-(3,3-difluoro-(4R)-hydroxy-(5R)-hydroxymethyl-tetrahydro-furan-2-yl)-2-oxo-1,2-dihydro-pyrimidin-4-ylcarbamoyloxy]-butyric acid was prepared from DFDC and (2R)-[3-Cyclobexyl-(2S)-(2-trimethylsilany]-etboxycarbonylamino)-propionylamino]-(3S)-hydroxy-butyric acid 2-trimethylsilanyl-ethyl ester by the Method Similar to Example 6

¹H-NMR:(270 MHz, CD₃OD): δ0.89-1.05 (2H, m), 1.16-1.25 (2H, m), 1.40-1.82 (9H, m), 1.32 (3H, d, J=6.3), 3.80 (1H, dd, J=2.9, 12.5), 3.93-4.05 (3H, m), 4.30, (1H, dq, J=4.3, 8.3), 4.44, (1H, d, J=3.9), 5.42 (1H, dt, J=2.0, 4.3), 6.24 (1H 7.31 (1H, d, J=7.6), 8.33 (1H, d, J=7.6 Hz); LC-MS: m/z 561.9 [M+H]⁺.

### Example 19

(S)-[2(S)-A-mino-3-cyclohexyl-propionylamino)-3-[1-(3,3-difluoro-4(R)-hydroxy-5(R)-hydroxymethyl-tetrahydro-furan-2(R)-yl)-2-oxo-1,2-dihydro-pyrimidin-4-ylcarbamoyloxy]-2(S)-methyl-propionic acid

To a stirred solution of 255.1 mg (0.514 mmol) of 2(5)-[2(S)-benzyloxycarbonylamino-3-cyclohexyl-propionylamino]-3-hydroxy-2(S)-methyl-propionic acid benzyl ester in 10.0 mL of CH₂Cl₂ (dehydrated) was added 207 mg (1.028 mmol) of 4-nitrophenyl chloroformate and 83 ml of pyridine at room temperature.

After stirring for 1.5 hrs, the reaction was quenched by addition of water, and organic layer was separated. The aqueous layer was extracted with EtOAc. The combined organic layer was washed with water and brine. The extract was dried over anhydrous Na₂SO₄ and filtered.

The solvent was removed under reduced pressure.

The crude product was purified by flash chromatography on SiO₂ (eluent: 20% EtOAc/hexane) to give 2(S)-[2(S)-benzyloxycarbonylamino-3-cyclohexyl-propionylamino]-2(S)-methyl-3-(4-nitro-phenoxycarbonyloxy)-propionic acid benzyl ester as a pale yellow solid (342.3 mg, quantity; including some p-nitrophenol).

¹H-NMR: (270 MHz, CDCl₃) δ0.97(2H, m), 1.13-1.50 (5H, m), 1.61(3H, s), 1.62-1.77 (6H, m), 4.20(1H, m), 4.66(1H, d, J=10.9 Hz, AB), 4.92(1H, d, J=10.9 Hz, AB), 4.98(1H, br.d), 5.08(2H, m), 5.22(2H, m), 6.92(1H, br.s), 7.30(2H, d, J=9.6 Hz), 7.34(10H, s), 8.22(2H, d, J=9.6 Hz); MS: (LCMS) m/z 662 [M+H]⁺.

To a stirred solution of 337 mg (0.509 mmol) of 2(S)-[2(S)-benzyloxy-carbonylamino-3-cyclohexyl-propionylamino]-2(S)-methyl-3-(4-nitro-phenoxy-carbonyloxy)-propionic acid benzyl ester in 5 mL of THF(dehydrated) was added 310 mg(0.611 mmol) of 3′,5′-di-O-tert-butyldimethylsilyl-DFDC at room temperature.

The reaction mixture was warmed up to 60° C. in an oil bath. After stirring for 18 hrs, the mixture was cooled to room temperature and the mixture was concentrated under reduced pressure. The oily residue was dissolved in EtOAc and washed with sat. NaHCO₃, water and brine. The organic layer was dried over anhydrous Na₂SO₄ and filtered. The solvent was removed under reduced pressure. The crude product was purified by flash chromatography on SiO₂ (eluent: 20% to 40% EtOAc/hexane) to give a coupled product as a colorless solid (437.7 mg, 85%). Successively, this product (106 mg; 0.105 mmol) was dissolved in 10 ml of THF (dehydrated) and then this was added 200 mL of n-tetrabutylammonium fluoride (1 mol/L in THF) at room temperature.

After stirring for 2 hr, the solvent was removed under reduced pressure, the yellowish oily residue was purified by flash chromatography on SiO₂ (eluent: 70% EtOAc to 100% EtOAc) to afford 2(S)-[2(S)-benzyloxycarbonylamino-3-cyclohexyl-propionylamino)-3-[1-(3,3-difluoro-4(R)-hydroxy-5(R)-hydroxymethyl-tetrahydro-furan-2(R)-yl)-2-oxo-1,2-dihydro-pyrimidin-4-ylcarbamoyloxy]-2(S)-methyl-propionic acid benzyl ester as a colorless solid (67.9 mg, 82%).

¹H-NMR: (270 MHz, CD₃OD) δ0.75(2H, m), 1.03-1.37(4H, m), 1.43(3H, s), 1.52-1.62(7H, m), 3.79(2H, m), 3.85 (1H, m), 4.13(1H, m), 4.19(2H,m), 4.80(1H, br.s) 4.90(1H, d, J=12.5 Hz, AB), 4.99(1H, d, J=12.5 Hz, AB), 5.02(2H, s), 6.16(1H,dd, J=7.9, 6.9 Hz), 7.19(1H, d, J=7.6 Hz), 7.20(5H, s), 7.21(5H, s), 8.21(1H, d, J=7.6 Hz); MS: (LCMS) m/z 786 [M+H]⁺.

To a solution of 62.1 mg (0.079 mmol) of 2(S)-[2(S)-benzyloxycarbonylamino-3-cyclohexyl-propionylamino)-3-[1-(3,3-difluoro-4(R)-hydroxy-5(R)-hydroxymethyl-tetrahydro-furan-2(R)-yl)-2-oxo-1,2-dihydro-pyrimidin-4-ylcarbamoyloxy]-2(S)-methyl-propionic acid benzyl ester in 5 mL of MeOH was added 10% Pd/C.

The reaction mixture was stirred vigorously under H₂ atmosphere. After stirring for 15 min., the mixture was filtered through a short pad Celite column. The filtrate was concentrated under reduced pressure and crude product was purified by preparative HPLC (ODS) to give 2(S)-[2(S)-amino-3-cyclohexyl-propionylamino)-3-[1-(3,3-difluoro-4(R)-hydroxy-5(R)-hydroxymethyl-tetrahydro-furan-2(R)-yl)-2-oxo-1,2-dihydro-pyrimidin-4-ylcarbamoyloxy]-2(S)-methyl-propionic acid as a colorless solid (35.8 mg, 81%).

HPLC condition: column; 5×30 cm (TSK-gel 80-TS ODS), eluent; 5% MeCN/H₂O to 100% MeCN (40 min. liner gradient), flow rate: 50 mL/min., detection: photodiode array.

¹H-NMR: (270 MHz, CD₃OD) δ0.90(2H, m), 1.01-1.34(4H, m), 1.50(3H, s), 1.54-1.75(7H, m), 3.76(2H, m), 3.95 (2H, m), 4.27(1H, dd, J=12.2, 8.2 Hz), 4.48(1H, d, J=10.9 Hz, AB), 4.92(1H, d, J=10.9 Hz, AB), 6.25(1H,dd, J=7.6, 6.9 Hz), 7.28(1H, d, J=7.6 Hz), 8.30(1H, d, J=7.6 Hz); MS: (LCMS) m/z 562 [M+H]⁺.

The following compounds in examples 20-22 were prepared from DFDC using a different dipeptide derivative of formula (VIII) by the method similar to Example 19.

### Example 20

2(R)-[2(S)-Amino-3-cyclohexyl-propionylamino]-3-{1-[3,3-difluoro-4(R)-hydroxy-5(R)-hydroxymethyl-tetrahydro-furan-2(R)-yl]-2-oxo-1,2-dihydro-pyrimidin-4-ylcarbamoyloxy}-2(R)-methyl-propionic acid was prepared from DFDC and 2(R)-[2(S)-Benzyloxycarbonylamino-3-cyclohexyl-propionylamino]-3-hydroxy-2(R)-methyl-propionic acid benzyl ester

¹H-NMR: (270 MHz, CD₃OD) δ0.90(2H, m), 1.02-1.45(4H, m), 1.55(3H, s), 1.62-1.75(7H, m), 3.80(1H, m), 3.93 (3H, m), 4.21(1H, m), 4.27(1H, d, J=10.5 Hz,5.00(1H, d, J=10.5 Hz, AB), 6.24(1H,dd, J=7.6, 7.3 Hz), 7.27(1H, d, J=7.6 Hz), 8.28 (1H, d, J=7.6 Hz); MS: (LCMS) m/z 562 [M+H]⁺.

### Example 21

(2S, 3S)-2-(2-Amino-3-cyclohexyl-propionylamino)-3-[1-{(4R, 5R)-3,3-difluoro-4-hydroxy-5-hydroxylmethyl-tetrahydro-furan-2-yl}-2-oxo-1,2-dihydro-pyridine-4-ylcarbamoyloxy]-2-methyl-butyric acid was prepared from DFDC and (2S, 3S)-2-(2-Benzyloxycarbonylamino-3-cyclohexyl-propionylamino)-3-hydroxy-2-methyl-butyric acid benzyl ester

¹H-NMR: (270 MHz, CD₃OD) δ0.91-1.70 (13H, m), 1.34 (3H, d, J=6.3 Hz), (3H, s), 3.73-3.94 (4H, m), 4.25 (1H, td, J=12.2, 8.6 Hz), 5.50 (1H, q, J=6.6 Hz), 6.20(1H,t,J=7.3 Hz), 7.19 (1H, d, J=7.6 Hz), 8.25 (1H, d, J=7.6Hz); MS: (LC-MS) m/z 576 [M+H]⁺.

### Example 22

(2R, 3R)-2-(2-Amino-3-cyclohexyl-propionylamino)-3-[1-{(4R, 5R)-3,3-difluoro-4-hydroxy-5-hydroxylmethyl-tetrahydro-furan-2-yl-2-oxo-1,2-dihydro-pyridine-4-ylcarbamoyloxy]-2-methyl-butyric acid was prepared from DFDC and (2R, 3R)-2-(2-Benzyloxycarbonylamino-3-cyclohexyl-propionylamino)-3-hydroxy-2-methyl-butyric acid benzyl ester

¹H-NMR: (270 MHz, CD₃OD) δ0.77-1.75 (13H, m), 1.42 (3H, d, J=6.6 Hz), (3H, s), 3.77-3.99 (4H, m), 4.27 (1H, td, J=12.2, 8.3 Hz), 5.54 (1H, q, J=6.6 Hz), 6.26 (1H, dd, J=7.6, 7.3 Hz), 7.31 (1H, d, J=7.6 Hz), 8.29 (1H, d, J=7.6 Hz); MS: (LC-MS) m/z 576 [M+H]³⁰ .

### Example 23

2R-(2S-amino-3-cyclohexyl-propionylamino)-3S-[1-(4S-hydroxy-5R-hydroxymethyl-3-methylene-tetrahydro-furan-2R-yl)-2-oxo-1,2-dihydro-pyrimidine-4-ylcarbamoyloxyl-butyric acid isopropyl ester

a) To a stirred solution of 5.5 g (17.8 mmol) BOC-D-Thr(Bzl)-OH, 280 mg (2.3 mmol) DMAP and 2.7 ml (35.6 mmol) 2-propanol in 50 ml dichloromethane (dehydrated) was added 4.41 g (23.1 mmol) WSC HCl at 0° C. The mixture was stirred for 5 hrs under Ar at ambient temperature. The reaction was quenched with 300 ml water, and the organic layer was separated. The aqueous layer was extracted with EtOAc (300 ml×2). The combined organic layer was washed with water (300 ml) and brine (300 ml), dried over anhydrous Na₂SO₄ and filtered. The solvent was removed under reduced pressure. The crude product was purified by a column of silica gel (100 g, eluent: 20% EtOAc/n-hexane) to give 3S-benzyloxy-2R-tert-butoxycarbonylamino-butyric acid isopropyl ester as colorless syrup (6.372 g, quant.).

¹H-NMR: (270 MHz, CDCl₃) δ1.10-1.30 (9H, m), 1.43(9H, s), 4.03-4.26(2H, m), 4.34 (1H, d, J=11.6), 4.51 (1H, d, J=11.6), 4.99 (1H, heptet, J=6.6), 5.24 (1H, br.d, J=8.9), 7.11-7.35 (5H, m); MS: (LCMS) m/z 373.9 (M+Na).

b) To a solution of 6.372 g (18.1 mmol) 3S-benzyloxy-2R-tert-butoxycarbonylamino-butyric acid isopropyl ester in 200 ml EtOAc was suspended 10% Pd/C and stirred vigorously for 3 hrs under H₂ atmosphere. The catalyst was filtered off and thoroughly washed with EtOAc. The filtrate was concentrated under reduced pressure to give 2R-tert-Butoxycarbonylamino-3S-hydroxy-butyric acid isopropyl ester as colorless syrup (4.74 g, quant.). The product was used in the next reaction step without further purification.

¹H-NMR: (270 MHz, CDCl₃) δ1.16 (6H, d, J=6.3), 1.23 (3H, d, J=6.3), 1.43 (9H, s), 2.05 (1H, br.s), 4.14-4.25 (2H, m), 5.06 (1H, heptet, J=6.3), 5.27 (1H, d, J=4.3); MS: (LCMS) m/z 262.1 [M+H]⁺.

c) To a solution of 4.74 g (18.1 mmol) 2R-tert-butoxycarbonylamino-3S-hydroxy-butyric acid isopropyl ester in 50 mL EtOAc was added 18 ml 4N HCl in EtOAc at room temperature. The mixture was stirred for 14 hrs and concentrated under reduced pressure to afford 2R-amino-3S-hydroxy-butyric acid isopropyl ester hydrochloride as colorless syrup (3.60 g, quant). The product was used next reaction step without further purification.

¹H-NMR: (270 MHz, DMSO-d₆) δ1.21 (6H, d, J=6.6), 1.25 (3H, d, J=6.3), 3.83 (1H, d, J=4.0), 4.05-4.15 (1H, m), 5.00 (1H, heptet, J=6.3), 5.65 (1H, d, J=5.3), 8.40 (3H, br.s); MS: (LCMS) m/z 162.0 [M+H]⁺.

d) A solution of 3.7g (17.8 mmol) 3-cyclohexyl-2S-amino-propionic acid hydrochloride, 6.3 g (18.7 mmol) FmocOSu and 2.47 ml (21.5 mmol) triethylamine in 30 ml dioxane and 15 ml water was stirred for 8 hrs at ambient temperature. The reaction mixture was concentrated under reduced pressure and the residual material was partitioned between EtOAc (200 ml) and 0.1 N aqueous citrate. The aqueous layer was extracted with EtOAc (200 ml). The combined organic layer was washed with water (100 ml), dried over anhydrous Na₂SO₄ and filtered through a glass filter. The filtrate was concentrated under reduced pressure and the residual solid was triturated from 20% EtOAc/n-hexane (100 ml) to give 3-cyclohexyl-2S-(9H-fluoren-9-ylmethoxycarbonylamino)-propionic acid as colorless crystals (6.8 g, 97%).

¹H-NMR: (270 MHz, DMSO-d₆) δ0.76-0.96 (2H, m), 1.10-1.20 (4H, m), 1.25-1.35 (1H, m), 1.50-1.70 (6H, m), 4.00 (1H, dd, J=8.9, 5.6), 4.21-4.30 (2H, m), 7.32 (2H, t, J=7.6), 7.41 (2H, t, J=7.6), 7.64 (1H, d, J=8.3), 7.90 (2H, d, J=7.3), 12.5 (1H, s); MS: (LCMS) m/z 393.9 [M+H]⁺.

e) To a stirred suspension of 6.8 g (17.3 mmol) 3-cyclohexyl-2S-(9H-fluoren-9-ylmethoxycarbonylamino)-propionic acid and 2.0 g (17.3 mmol) N-hydroxysuccinimide in 60 ml 50% dioxane/EtOAc was added 3.92 g dicyclohexylcarbodiimide in one portion at 0° C. The reaction mixture was stirred for 6 hrs at room temperature. The precipitates were filtered off on a grass filter and washed thoroughly with EtOAc. The filtrate was concentrated under reduced pressure to give the crude N-hydroxysuccinimide ester. The residue was dissolved in 100 ml dichloromethane and 3.52 g (17.8 mmol) 2-R-amino-3S-hydroxy-butyric acid isopropyl ester hydrochloride and 5.18 ml (37.4 mmol) were added and stirred for 9 hrs at ambient temperature. The reaction was quenched with 0.1 N aqueous citrate (100 ml)and the organic layer was separated. The aqueous layer was extracted with EtOAc (200 ml×2) and the combined organic layer was washed with brine (100 ml), dried over anhydrous Na₂SO₄, and concentrated under reduced pressure. The residual material was recrystalized from 20% EtOAc/n-hexane to give 2R-[3-cyclohexyl-2S-(9H-fluoren-9-ylmethoxycarbonylamino)-propionylamino]-3S-hydroxy-butyric acid isopropyl ester as colorless crystals (8.432 g, 91%).

¹H-NMR: (270 MHz, DMSO-d₆) δ0.86-0.96 (2H, m), 1.03 (3H, d, J=6.3), 1.00-1.40 (11H, m), 1.50-1.75 (6H, m), 4.08-4.30 (5H, m), 4.90 (1H, heptet, J=5.6), 4.97 (1H, d, J=5.6), 7.28-7.53 (4H, m), 7.60-7.78 (3H, m), 7.90 (2H, d, J=7.3); MS: (LCMS) m/z 537.0 [M+H]⁺.

f) To a stirred solution of 8.40 g (15.7 mmol) 2R-[3-cyclohexyl-2S-(9H-fluoren-9-ylmethoxycarbonylamino)-propionylamino]-3S-hydroxy-butyric acid isopropyl ester in 200 mL of dichloromethane (dehydrated) were added 8.2 g (4.1 mmol) 4-nitrophenyl chloroformate and 3.29 mL pyridine at room temperature.

After stirring for 2 hrs, the reaction was quenched by addition of water, and organic layer was separated. The aqueous layer was extracted with EtOAc (200 ml). The combined organic layer was washed with water (200 ml×2) and brine (200 ml), dried over anhydrous Na₂SO₄ and concentrated under reduced pressure. The crude product was recrystalized from EtOAc and n-hexane to give 2R-[3-cyclohexyl-2S-(9H-fluoren-9-ylmethoxycarbonylamino)-propionylamino]-3S-(4-nitro-phenoxycarbonyloxy-butyric acid isopropyl ester as colorless crystals (10.6 g, 96%).

¹H-NMR: (270 MHz, DMSO-d₆) δ0.86-0.96 (2H, m), 1.29 (3H, d, J=6.3), 1.00-1.40 (11H, m), 1.50-1.75 (6H, m), 4.21-4.35 (5H, m), 4.68 (1H, dd, J=4.3, 8.6), 4.93 (1H, heptet, J=6.3), 5.26 (1H, m), 7.29-7.55 (6H, m), 7.60-7.78 (2H, m), 7.90 (2H, d, J=7.3), 8.31 (2H, dd, J=2.3, 6.9), 8.54 (1H, d, J=8.6); MS: (LCMS) m/z 702.1 [M+H]⁺.

g) A solution of 5.0 g (7.0 mmol) 2R-[3-cyclohexyl-2s-(9H-fluoren-9-ylmethoxycarbonylamino)-propionylamino]-3S-(4-nitro-phenoxycarbonyloxy-butyric acid isopropyl ester and 3.8 g (8.12 mmol) 3′,5′-di-tert-butyldimethylsilyl-DMDC in 40 mL THF(dehydrated) was stirred for 2 days at 70° C. The mixture was concentrated under reduced pressure. The oily residue was partitioned between EtOAc (150 ml×2) and sat. NaHCO₃, solution. The combined organic layer was washed with water (100 ml) and brine (100 ml×2), dried over anhydrous Na₂SO₄ and concentrated under reduced pressure. The crude product was purified by a column of silica gel (eluent: 25% EtOAc/n-hexane) to give 2R-[3-cyclohexyl-2S-(9H-fluoren-9-ylmethoxycarbonylamino)-propionylamino]-3S-{1-[4S-(tert-butyldimethylsilanyloxy)-5R-(tert-butyldimethylsilanyloxymethyl)-3-methylene-tetrahydro-furan-2-R-yl]-2-oxo-1,2-dihydro-pyrimidine-4-ylcarbamoyloxy}-butyric acid isopropyl ester as colorless amorphous (6.6 g, 90%).

¹H-NMR: (270 MHz, DMSO-d₆) δ0.06 (3H, s), 0.06 (3H, s), 0.07 (6H, s), 0.83 (9H, s), 0.88 (9H, s), 0.83-1.81 (22H, m), 3.70-3.80 (2H, m), 3.82 (1H, d, J=6.8), 4.15-4.25 (4H, m), 4.54 (1H, dd, J=4.3, 8.6), 4.74 (1H, d, J=5.3), 4.86 (1H, heptet, J=6.3), 5.23 (1H, m), 5.29 (1H, s), 5.37 (1H, s), 6.57 (1H, s), 6.94 (1H, d, J=5.0), 7.29 (2H, t, J=7.3), 7.39 (2H, t, J=7.3), 7.61 (1H, d, J=8.3), 7.73 (2H, dd, J=3.3, 7.6), 7.87 (2H, t, J=7.3), 7.98 (2H, m); MS: (LCMS) m/z 1030.3 [M+H]⁺.

h) To a solution of 200 mg (0.194 mmol) of 2R-[3-cyclohexyl-2S-(9H-fluoren-9-ylmethoxycarbonylamino)-propionylamino]-3S-{1-[4S-(tert-butyldimethylsilanyloxy)-5R-(tert-butyldimethylsilanyloxymethyl)-3-metylene-tetrahydro-furan-2R-yl]-2-oxo-1,2-dihydro-pyrimidine-4-ylcarbamoyloxy}-butyric acid isopropyl ester in 3 mL THF(dehydrated) was added 323 μL (1.941 mmol) HF triethylamine (98%) at room temperature. After stirring for 14 brs, the reaction mixture was concentrated under reduced pressure and the residue was purified by a column of silica gel (eluent: 6.25% methanol/dichloromethane) to give 2R-[3-cyclohexyl-2S-(9H-fluoren-9-ylmethoxycarbonylamino)-propionylamino]-3S-[1-(4S-hydroxy-5R-hydroxymethyl-3-methylene-tetrahydro-furan-2R-yl)-2-oxo-1,2-dihydro-pyrimid 4-ylcarbamoyloxy]-butyric acid isopropyl ester as colorless amorphous(145.7 mg, 94%).

¹H-NMR: (270 MHz, DMSO-d₆) δ0.80-0.99 (2H, m), 1.10-1.81 (20H, m), 3.55-3.80 (3H, m), 4.15-4.30 (4H, m), 4.50 (1H, m), 4.58 (1H, dd, J=3.3, 8.9), 4.86 (1H, heptet, J=6.3), 5.01 (1H, m), 5.20 (1H, m), 5.30 (1H, s), 5.34 (1H, s), 5.66 (1H, br.d), 6.53 (1H, s), 6.90 (1H, d, J=7.6), 7.30 (2H, t, J=7.3), 7.39 (2H, t, J=7.2), 7.65 (1H, d, J=8.2), 7.72 (2H, dd, J=3.3, 7.6), 7.88 (2H, d, J=7.3), 7.94 (1H, d, J=8.2), 8.10 (1H, d, J=7.6);

MS: (LCMS) m/z 802.0 [M+H]⁺.

i) To a solution of 136 mg (0.17 mmol) 2R-[3-cyclohexyl-2S-(9H-fluoren-9-ylmethoxycarbonylamino)-propionylamino]-3S-[1-(4S-hydroxy-5R-hydroxymethyl-3-methylene-tetrahydro-furan-2R-yl)-2-oxo-1,2-dihydro-pyrimidine-4-ylcarbamoyloxy]-butyric acid isopropyl ester in 1 ml DMF (dehydrated) was added 100 μL piperidine at room temperature.

After stirring for 3 hrs, the solvent was removed under reduced pressure. The yellowish residue was purified by a column of silica gel (eluent: 10% methanol/dichloromethane) to give 2R-(2S-amino-3-cyclohexyl-propionylamino)-3S-[1-(4S-hydroxy-5R-hydroxymethyl-3-methylene-tetrahydro-furan-2R-yl)-2-oxo-1,2-dihydro-pyrimidine-4-ylcarbamoyloxy]-butyric acid isopropyl ester as a colorless solid (28.6 mg, 29%).

¹H-NMR: (270 MHz, DMSO-d₆) δ0.75-0.95 (2H, m), 1.12-1.80 (20H, m), 3.55-3.80 (3H, m), 4.50 (1H, m), 4.60 (1H, m), 4.88 (1H, heptet, J=6.3), 5.03 (1H, m), 5.30-5.35 (4H, m), 5.70 (1H, br.d), 6.53 (1H, s), 6.93 (1H, d, J=7.6), 8.06 (1H, br.s), 8.10 (1H, d, J=7.6);

MS: (LCMS) m/z 579.9 [M+H]⁺.

### Reference Example 2.1

Preparation of (20S)-9-nitrocamptothecin-N-oxide 20-acetate

To a solution of 9-nitrocamptothecin 20-acetate (8.62 g, 19.8 mmol) in trifluoroacetic acid (65 ml) was added urea-hydrogen peroxide (3.11 g, 33.1 mmol) at room temperature. After stirring for 4 hr. at room temperature, the mixture was concentrated under reduce pressure to approximately a half volume and poured into an ice-water mixture. The generated precipitate was collected by filtration, washed with distilled water, and dried in vacuo to obtain the titled compound (8.35 g, 93% yield).

¹H NMR (270 MHz) δ (CDCl₃) 0.98 (t, J=7.6 Hz, 3H), 2.08-2.33 (m, 2H), 2.23 (s, 3H), 5.38 (s, 2H), 5.40 (d. J=17.7 Hz, 1H), 5.67 (d, J=17.7 Hz, 1H), 7.96 (s, 1H), 7.96 (dd, J=7.6 and 7.8 Hz, 1H), 8.67 (s, 1H), 9.16 (d, J=7.6 Hz, 1H); MS m/z (ES) 452 (M⁺+1).

### Reference Example 3.1

Preparation of (20S)-7-chloro-9-nitrocamptothecin 20-acetate

To a solution of (20S)-9-nitrocarnptothecin-N-oxide 20-acetate (10.88 g, 24.1 mmol) of Reference Example 2.1 in N,N-dimethylformamide (196 ml) was added oxalyl chloride (4.2 ml, 48.2 mmol) at 0° C., and the mixture was stirred at 15° C. for 3 hr. The mixture was poured into ice-water (500 ml), and extracted with ethyl acetate (500 ml×1; 250 ml×2). The organic layer was dried over anhydrous sodium sulfate, and concentrated under reduced pressure. The residue was purified by silica gel column chromatography (ethyl acetate/hexane=1/1) to give the titled compound (5.54 g, 49%) as a yellow solid.

¹H NMR (270 MHz) δ (CDCl₃) 0.99 (t, J=7.6 Hz, 3H), 2.07-2.33 (m, 2H), 2.23 (s, 3H), 5.33 (s, 2H), 5.41 (d, J=17.8 Hz, 1H), 5.69 (d, J=17.8 Hz, 1H), 7.20 (s, 1H), 7.87-7.95 (m, 2H), 8.44 (dd, J=2.3 and 7.6 Hz, 1H); MS m/z (ES) 470 (M⁺+1).

### Reference Example 4.1

Preparation of (20S)-9-nitro-7-(pentylamino)camptothecin 20-acetate

To a suspension of (20S)-7-chloro-9-nitrocamptothecin 20-acetate (2.58 g, 5.49 mmol) of Reference Example 3.1 in 1,4-dioxane (29 ml) was added n-amylamine (2.55 ml, 21.96 mmol) and the mixture was stirred at 80° C. for 2 hr, followed by concentration under reduced pressure. The resulting residue was purified by silica gel column chromatography (dichloromethane/acetone=30/1−20/1) to give the titled compound (1.80 g, 63%) as a brown oil.

¹H NMR (270 MHz) δ (CDCl₃) 0.86-1.01 (m, 6H), 1.22-1.59 (m, 4H), 1.60-1.78 (m, 2H), 2.03-2.37 (m, 5H), 3.57-3.68 (m, 2H), 5.02 (br, 1H), 5.40 (d, J=17.2 Hz, 1H), 5.47 (s, 2H), 5.67 (d, J=17.2 Hz, 1H), 7.13 (s, 1H), 7.66 (dd, J=2.0, 7.9 Hz, 1H), 7.71 (t, J=7.9 Hz, 1H), 8.23 (dd, J=2.0, 7.9 Hz, 1H); MS (ES) m/z 521 (M⁺+1).

### Reference Example 4.15:

Preparation of (20S)-7-butylamino-9-nitrocamptothecin 20-acetate

This compound was prepared from (20S)-7-chloro-9-nitrocamptothecin 20-acetate of Reference Example 3.1 and butylamine according to a manner analogous to those of Reference Example 4.1.

¹H NMR (270 MHz) δ (CDCl₃) 0.97 (t, J=7.6 Hz, 3H), 1.00 (t, J=7.3 Hz, 3H), 1.43-1.52 (m, 2H), 1.63-1.71 (m, 2H), 2.13-2.32 (m, 2H), 2.22 (s, 3H), 3.62-3.69 (m, 2H), 5.02 (brt, 1H), 5.40 (d, J=17.2 Hz, 1H), 5.47 (s, 2H), 5.66 (d, J=17.2 Hz, 1H), 7.14 (s,1H), 7.65-7.74 (m, 2H), 8.23 (dd, J=1.6 and 7.9 Hz, 1H); MS m/z (ES) 507 (M⁺+1).

### Reference Example 5.1

Preparation of (20S)-9-amino-7-(butylamino)camptothecin 20-acetate

(20S)-7-butylamino-9-nitrocamptothecin 20-acetate (156 mg, 0.31 mmol) of Reference Example 4.15 was dissolved into MeOH (10 ml) and 1N HCl aqueous solution (2 ml). 5% Pd—C (15 mg) was added and the hydrogenation was carried out under H₂ atmosphere using a balloon at room temperature for 1 hr. After removing Pd—C by filtration, the filtrate was concentrated under reduced pressure to obtain the product (137 mg, 87% yield).

¹H NMR (270 MHz) δ (CDCl₃) 0.95 (t, J=7.6 Hz, 3H), 1.01 (t, J=7.3 Hz, 3H), 1.48-1.60 (m, 2H), 1.68-1.78 (m, 2H), 2.10-2.31 (m, 2H), 2.20 (s, 3H), 3.60-3.67 (m, 2H), 3.90 (brs, 2H), 5.39 (d, J=17.0 Hz, 1H), 5.41 (s, 2H), 5.66 (d, J=17.0 Hz, 1H), 6.85 (d, J=7.3 Hz, 1H), 7.11 (s, 1H), 7.45 (dd, J=7.3 and 8.3 Hz, 1H), 7.64 (d, J=8.3 Hz, 1H), 8.77 (brs, 1H); MS (ES) m/z 477 (M⁺+1).

### Reference Example 5.14

Preparation of (20S)-9-amino-7-(pentylamino)camptothecin 20-acetate hydrochloride

This compound was prepared from (20S)-9-nitro-7-(pentylamino)camptothecin 20-acetate of Reference Example 4.1 according to a manner analogous to those of Reference Example 5.1.

MS (ES) m/z 491 (M⁺+1).

### Example 1.1

Preparation of (9S)-1-butyl-9-ethyl-9-hydroxy-1H,12H-pyrano[3″,4″:6′,7′]indolizino-[1 ′,2′:6,5]pyrido[4,3,2-de]quinazoline-2,10,13(3H,9H, 15H)-trione

The preparation method comprises of the following two steps via compound (a).

(a) (9S)-9-acetoxy-1-butyl-9-ethyl-1H,12H-pyrano[3″,4″:6′,7′]indolizino[1′,2′:6,5]-pyrido [4,3,2-de]quinazoline-2,10,13(3H,9H, 15H)-trione

(20S)-9-amino-7-(butylamino)camptothecin 20-acetate hydrochloride (123 mg, 0.24 mmol) of Reference Example 5.1 was dissolved into dry CH₂Cl₂ (5 ml) and cooled in an ice-bath. DIEA (390 μl, 2.3 mmol) and triphosgene (67 mg, 0.23 mmol) were added successively and the mixture was stirred for 1 hr. in the ice-bath. The reaction mixture was quenched with aqueous 1N HCl solution at 0° C., and extracted with CH₂Cl₂ (20 ml). The CH₂Cl₂ layer was washed with brine, dried over MgSO₄, and evaporated under reduced pressure. The residue obtained was purified by column chromatography (dichloromethane/acetone=15/1-7/1) to give a pure product (70 mg, 56%).

¹H NMR (270 MHz) δ (CDCl₃) 0.98 (t, J=7.7 Hz, 3H), 1.00 (t, J=7.3 Hz, 3H), 1.43-1.59 (m, 2H), 1.66-1.77 (m, 2H), 2.07-2.35 (m, 2H), 2.23 (s, 3H), 4.12-4.18 (m, 2H), 5.38 (s, 2H), 5.40 (d, J=17.4 Hz, 1H), 5.68 (d, J=17.4 Hz, 1H), 6.76 (dd, J=1.5 and 6.7 Hz, 1H), 7.16 (s, 1H), 7.56-7.67 (m, 2H), 9.24 (s, 1H); MS (ES) m/z 503(M⁺+1).

(b) (9S)-1-butyl-9-ethyl-9-hydroxy-1H,12H-pyrano[3″,4″:6′,7′]indolizino[1′,2′:6,5]-pyrido[4,3,2-de]quinazoline-2,10,13(3H,9H,15H)-trione

To a solution of (9S)-9-acetoxy-1-butyl-9-ethyl-1H,12H-pyrano[3″,4″:6′,7′]indolizino[1′,2′:6,5]pyrido[4,3,2-de]quinazoline-2,20,13(3H,9H,15H)-trione (11.5 mg, 0.023 mmol) in MeOH (3 ml) cooled in an ice-bath was added anhydrous hydrazine (100 μl). The mixture was warmed to room temperature and stirred for 1 hr. Aqueous 1 N HCl solution was added dropwise to acidify the reaction mixture and the mixture was stirred for 1 hr. at room temperature. After concentrated under reduced pressure, the resulting residue was extracted with CH₂Cl₂ (20 ml×3). The combined CH₂Cl₂ solution was washed with brine, dried over MgSO₄ and evaporated. The residue was purified by column chromatography (dichloromethane/methanol=30/1) to give pure product (6.1 mg, 58%).

¹H NMR (400 MHz) δ (DMSO) 0.87 (t, J=7.2 Hz, 3H), 0.96 (t, J=7.6 Hz, 3H), 1.39-1.47 (m, 2H), 1.64-1.70 (m, 2H), 1.81-1.91 (m, 2H), 4.03-4.07 (m, 2H), 5.42 (s, 2H), 5.43 (s, 2H), 6.51 (s, 1H), 6.77 (d, J=7.2 Hz, 1H), 7.24 (s, 1H), 7.41 (d, J=7.6 Hz, 1H), 7.61 (dd, J=7.2 and 7.6 Hz, 1H), 11.15 (brs, 1H); MS (ES) m/z 461(M⁺+1).

### Example 1.14

Preparation of (9S)-9-ethyl-9-hydroxy-1-pentyl-1H,12H-pyrano[3″,4″:6′,7′]indolizino-[1′,2′:6,5]pyrido[4,3,2-de]quinazoline-2,10,13(3H,9H,15H)-trione

This compound was prepared from (20S)-9-amino-7-pentylamino)camptothecin 20-acetate of Reference Example 5.14 according to a manner analogous to those of Example 1.1 in two steps via compound (a).

(a) (9S)-9-acetoxy-9-ethyl-1-pentyl-1H,12H-pyrano[3″,4″:6′,7′]indolizino-[1′,2′:6,5]pyrido[4,3,2-de]quinazoline-2,10,13(3H,9H,15H)-trione

¹H NMR (270 MHz) δ (CDCl₃) 0.92 (t, J=6.9 Hz, 3H), 0.98 (t, J=7.6 Hz, 3H), 1,29-1.53 (m, 4H), 1.65-1.76 (m, 2H), 2.12-2.30 (m, 5H), 3.75-4.17 (m, 2H), 5.36 (m, 2H), 5.40 (d, J=17.5 Hz, 1H), 5.68 (d, J=17.5 Hz, 1H), 6.75 (dd, J=1.7, 6.9 Hz, 1H), 7.15 (s, 1H), 7.58 (dd, J=1.7, 6.9 Hz, 1H), 7.64 (dd, J=6.9, 8.6 Hz, 1H), 8.88 (br, 1H); MS (ES) m/z 517 (M⁺+1).

(b) (9S)-9-ethyl-9-hydroxy-1-pentyl-1H,12H-pyrano[3″,4″:6′,7′]indolizino[1′,2′:6,5]-pyrido[4,3,2-de]quinazoline-2,10,13(3H,9H,15H)-trione

¹H NMR (270 MHz) δ (DMSO-d₆) 0.85-0.93 (m, 6H), 1.36-1.38 (m, 4H) 1.69-1.88 (m, 4H), 4.05 (m, 2H), 5.43 (s, 4H), 6.49 (s, 1H), 6.78 (d, J=8.0 Hz, 1H), 7.25 (s, 1H), 7.42 (d, J=8.0 Hz, 1H), 7.61 (t, J=8.0 Hz, 1H), 11.13 (br, 1H); MS (ES) m/z 475 (M⁺+1).

### Examples 2.1

Preparation of (9S)-1-butyl-9-ethyl-9-hydroxy-1H,12H-pyrano[3″,4″:6′,7′]indolizino-[1′,2′:6,5]pyrido[4,3,2-de]quinazoline-10,13(9H,15H)-dione

The preparation method comprises of the following two steps via compound (a).

(a) (9S)-9-acetoxy-1-butyl-9-ethyl-1H,12H-pyrano[3″,4″:6′,7′]indolizino[1′,2′:6,5]-pyrido[4,3,2-de]quinazoline-10,13(9H,15H)-dione

To a solution of (20S)-9-amino-7-(butylamino)camptothecin 20-acetate hydrochloride (14.9 mg, 0.029 mmol) of Reference Example 5.1 in dry CH₂Cl₂ (5 ml) were added trimethyl orthoformate (100 μl) and p-toluenesulfonic acid monobydrate (5 mg). The mixture was heated to reflux for 1 hr. in an oil bath. After cooling to room temperature, the mixture was washed with aqueous 1% NaHCO₃ solution and brine successively, dried over MgSO₄ and concentrated under reduced pressure. The obtaining residue was purified by column chromatography (eluent: dichloromethane/methanol=20/1) to give pure product (12.6 mg, 89%).

¹H NMR (400 MHz) δ (CDCl₃) 0.96 (t, J=7.6 Hz, 3H), 1.01 (t, J=7.4 Hz, 3H), 1.49-1.58 (m, 2H), 1.74-1.82 (m, 2H), 2.09-2.17 (m, 2H), 2.21 (s, 3H), 2.24-2.31 (m, 1H), 3.84 (t,J=7.4 Hz, 2H), 5.22 (d, J=17.8 Hz, 1H), 5.25 (d, J=17.8 Hz, 1H), 5.39 (d, J=17.2 Hz, 1H), 5.65 (d, J=17.2 Hz, 1H), 7.10 (s, 1H), 7.16 (d, J=7.2 Hz, 1H), 7.40 (s, 1H), 7.62 (d, J=8.4 Hz, 1H), 7.68 (dd, J=7.2 and 8.4 Hz, 1H); MS (ES) m/z 487(M⁺+1).

(a) (9S)-1-butyl-9-ethyl-9-hydroxy-1H,12H-pyrano[3″,4″:6′,7′]indolizino[1′,2′:6,5]-pyrido[4,3,2-de]quinazoline-10,13(9H,15H)-dione

To a solution of (9S)-9-acetoxy-1-butyl-9-etbyl-1H,12H-pyrano[3″,4″:6′,7′]-indolizino[1′,2′:6,5]pyrido[4,3,2-de]quinazoline-10,13(9H,15H)-dione (6.1 mg, 0.013 mmol) in MeOH (2 ml) cooled in an ice-bath was added anhydrous hydrazine (100 μl) and the mixture was stirred for 1 hr. at room temperature. Aqueous 1 N HCl solution was added dropwise to acidify the reaction mixture, and the mixture was stirred for 1 hr. at room temperature. After concentrated under reduced pressure, the residue was extracted with CH₂Cl₂ (30 ml) and the CH₂Cl₂ solution was washed with brine, dried over MgSO₄ and evaporated. The residue was purified by column chromatography (dichloromethane/methanol=20/1) to give pure product (3.9 mg, 70%).

¹H NMR (400 MHz) δ (DMDO-d₆) 1.02 (t, J=7.2 Hz, 6H), 1.50-1.59 (m, 2H), 1.76-1.93 (m, 4H), 3.82 (t, J=7.2 Hz, 2H), 3.88 (brs, 1H), 5.21 (s, 2H), 5.27 (d, J=16.2 Hz, 1H), 5.70 (d, J=16.2 Hz, 1H), 7.11 (dd, J=1.6 and 7.4 Hz, 1H), 7.37 (s, 1H), 7.51 (s,1H), 7.59-7.67 (m, 2H); MS (ES) m/z 445(M⁺+1).

### Example 2.15

Preparation of (9S)-9-ethyl-9-hydroxy-1-pentyl-1H,12H-pyrano[3″,4″:6′,7′]indolizino-[1′,2′:6,5]pyrido[4,3,2-de]quinazoline-10,13(9H,15H)-dione

This compound was prepared from (20S)-9-amino-7-(pentylamino)camptothecin 20-acetate of Reference Example 5.14 according to a manner analogous to those of Example 2.1 in two steps via compound (a).

(a) (9S)-9-acetoxy-9-ethyl-1-pentyl-1H,12H-pyrano[3″,4″:6′,7′]indolizino-[1′,2′:6,5]pyrido[4,3,2-de]quinazoline-10,13(9H,15H)-dione

¹H NMR (270 MHz) δ (CDCl₃) 0.91-0.99 (m, 6H), 1.26-1.58 (m, 4H), 1.74-1.82 (m, 2H), 2.09-2.31 (m, 5H), 3.83 (t, J=7.3 Hz, 2H), 5.23 (s, 2H), 5.39 (d, J=17.2 Hz, 1H), 5.65 (d, J=17.2 Hz, 1H), 7.09 (s, 1H), 7.17 (dd, J=1.5, 6.9 Hz, 1H), 7.40 (s, 1H), 7.62 (dd, J=1.5, 8.6 Hz, 1H), 7.68 (dd, J=6.9, 8.6 Hz, 1H);

(b) (9S)-9-ethyl-9-hydroxy-1-pentyl-1H,12H-pyrano[3″,4″:6′,7′]indolizino-[1′,2′:6,5]pyrido[4,3,2-de]quinazoline-10,13(9H,15H)-dione

¹H NMR (270 MHz) δ (DMSO-d₆) 0.85-0.92 (m, 6H), 1.35-1.38 (m, 4H), 1.75-1.93 (m, 4H), 3.89-3.94 (m, 2H), 5.29 (s, 2H), 5.40 (s, 2H), 6.46 (s, 1H), 6.99 (dd, J=1.0, 7.4 Hz, 1H), 7.18 (s, 1H), 7.47 (dd, J=1.0, 8.6 Hz, 1H), 7.62 (dd, J=7.4, 8.6 Hz, 1H), 7.86 (s, 1H); MS (ES) m/z 459 (M⁺+1).

### Example 2.28

Preparation of (9S)-9-ethyl-9-hydroxy-2-methyl-1-pentyl-1H,12H-pyrano[3″,4″:6′,7′]indolizino[1′,2′:6,5]pyrido[4,3,2-de]quinazoline-10,13(9H,15H)-dione

This compound was prepared from (20S)-9-amino-7-(pentylamino)camptothecin 20-acetate of Reference Example 5.14 and trimethyl orthoacetate according to a manner analogous to those of Example 2.1 in two steps via compound (a).

(a) (9S)-9-acetoxy-9-ethyl-2-methyl-1-pentyl-1H,12H-pyrano[3″,4″:6′,7′]indolizino-[1′,2′:6,5]pyrido[4,3,2-de]quinazoline-10,13(9H,15H)-dione

¹H NMR (270 MHz) δ (CDCl₃) 0.94 (t, J=6.9 Hz, 3H), 0.97 (t, J=7.3 Hz, 3H), 1.30-1.56 (m, 4H), 1.65-1.89 (m, 2H), 2.05-2.35 (m, 2H), 2.21 (s, 3H), 2.49 (s, 3H), 3.79-4.01 (m, 2H), 5.24 (brs, 2H), 5.39 and 5.66 (q, J=17.2 Hz, 1H×2), 7.04-7.12 (m, 1H), 7.08 (s, 1H), 7.52-7.71 (m, 2H); MS (ES) m/z 515 (M⁺+1).

(b) (9S)-9-ethyl-9-hydroxy-2-methyl-1-pentyl-1H,12H-pyrano[3″,4″:6′,7′]-indolizino[1′,2′:6,5]pyrido[4,3,2-de]quinazoline-10,13(9H,15H)-dione

¹H NMR (270 MHz) δ (DMSO-d₆) 0.87 (t, J=7.3 Hz, 3H), 0.91 (t, J=7.3 Hz, 3H), 1.30-1.60 (m, 4H), 1.66-1.94 (m, 4H), 2.45 (d, J=2.6 Hz, 3H), 3.93 (br, 2H), 5.23-5.44 (m, 2H), 5.41 (brs, 2H), 6.50 (brs, 1H), 6.89-7.00 (m, 1H), 7.19 (d, J=2.3 Hz, 1H), 7.38-7.49 (m, 1H), 7.62 (dt, J=3.6 and 7.9 Hz, 1H); MS (FAB) m/z 473 (M⁺+1)

### Example 3.1

Preparation of (9S)-9-ethyl-9-hydroxy-2-hydroxymethyl-1-pentyl-1H,12H-pyrano[3″,4″:6′7′]indolizino[1′2′:6,5]pyrido[4,3,2-de]quinazoline-10,13(9H,15H)-dione

The preparation method comprises the following two steps via compound (a).

(a) (9S)-9-acetoxy-2-acetoxymethyl-9-ethyl-1-pentyl-1H,12H-pyrano[3″,4″:6′,7′]-indolizino[1′,2′:6,5]pyrido[4,3,2-de]quinazoline-10,13(9H, 15H)-dione

To a solution of (20S)-9-amino-7-(pentylamino)camptothecin 20-acetate hydrochloride (1.61 g mg, 3.07 mmol) of Reference Example 5.14 in dry dichloromethane (120 ml) cooled in an ice-bath were added acetoxyacetyl chloride (4.3 ml) and diisopropylethylamine (1.07 ml) successively. After the addition, the mixture was warmed to room temperature and stirred for overnight. Water (50 ml) was added and the mixture was extracted with dichloromethane (100 ml). The dichloromethane layer was washed with brine, dried over MgSO₄ and concentrated under reduced pressure. The obtaining residue was purified by column chromatography (eluent: ethyl acetate/hexane=8/1) to give pure product (1.72, 98%).

¹H NMR (400 MHz) δ (CDCl₃) 0.91 (t, J=7.3 Hz, 3H), 0.97 (t, J=7.5 Hz, 3H), 1.31-1.48 (m, 4H), 1.70-1.82 (m, 2H), 2.08-2.30 (m, 2H), 2.22 (s, 3H), 2.25 (s, 3H), 3.86 (t, J=7.9 Hz, 2H), 5.04 (s, 2H), 5.26 (s, 2H), 5.39 (d, J=17.1 Hz, 1H), 5.66 (d, J=17.1 Hz, 1H), 7.13 (s, 1H), 7.19 (dd, J=2.0 and 6.6 Hz, 1H), 7.63-7.73 (m, 2H); MS (ES) m/z 573(M⁺+1).

(b) (9S)-9-ethyl-9-hydroxy-2-hydroxymethyl-1-pentyl-1H,12H-pyrano[3″,4″:6′,7′]-indolizino[1′,2′:6,5]pyrido[4,3,2-de]quinazoline-10,13(9H,15H)-dione

To a solution of (9S)-9-acetoxy-2-acetoxymethyl-9-ethyl-1-pentyl-1H,12H-pyrano[3″,4″:6′,7′]indolizino[1′,2′:6,5]pyrido[4,3,2-de]quinazoline-10,13(9H,15H)-dione (34 mg, 0.059 mmol) in methanol (3 ml) cooled in an ice-bath was added anhydrous hydrazine (100 μl) and the mixture was stirred for 2 hr. at room temperature. Aqueous 1 N hydrochloric acid solution (5 ml) was added dropwise to acidify the reaction mixture, and the mixture was stirred for 1 hr. at room temperature. The mixture was extracted with dichloromethane (50 ml) and the dichloromethane layer was washed with brine, dried over MgSO₄ and evaporated. The residue was purified by column chromatography (dichloromethane/methanol=25/1) to give pure product (19 mg, 65%).

¹H NMR (400 MHz) δ (DMSO-d₆) 0.87 (t, J=7.6 Hz, 3H), 0.90 (t, J=6.9 Hz, 3H), 1.32-1.45 (m, 4H), 1.74-1.90 (m, 4H), 4.04 (m, 2H), 4.43 (d, J=5.6 Hz, 2H), 5.36 (s, 2H), 5.41 (s, 2H), 5.79 (t, J=5.6 Hz, 1H), 6.50 (s, 1H), 7.03 (dd, J=1.0 and 7.3 Hz, 1H), 7.20 (s, 1H), 7.50 (dd, J=1.0 and 8.6 Hz, 1H), 7.66 (dd, J=7.3 and 8.6 Hz, 1H); MS (ES) m/z 489(M⁺+1).

### Example 24

Preparation of 20-O—[(S)-tryptophyl-γ-(S)-glutamyl]-20-(S)-camptothecin hydrochloride

a) To a stirred solution of 2.5 g (7.58 mmol) of L-glutamic acid α-t-butyl-γ-benzyl diester hydrochloride in 75 mL of dichloromethane was added 3.65 g (9.10 mmol) of N-α-Boc-L-tryptophan hydroxy succinimide and 1.59 mL (9.10 mmol) of N,N-diisopropylethylamine. The mixture was stirred over night under nitrogen atmosphere at room temperature. The reaction was quenched by addition of saturated ammonium chloride solution, and organic layer was separated. The aqueous layer was extracted with dichloromethane. The combined organic layer was washed with water and brine. The extract was dried over anhydrous magnesium sulfate and filtered. The solvent was removed under reduced pressure. The crude product was purified by medium pressure liquid chromatography with Lobar LiChroprep Si-60 Grobe C (eluent:ethyl acetate/dichloromethane=1/1) to give 2(S)-[2(S)-tert-butoxycarbonylamino-3-(1H-indol-3-yl)-propionylamino]-pentanedioic acid 5-benzyl ester 1-tert-butyl ester as a white amorphous (4.38 g, quant).

¹H-NMR: (270 MHz, CDCL₃) δ1.30-1.49(15H, m), 1.62 (3H, s), 1.73-1.95 (1H, m), 2.02-2.25 (3H, m), 3.13(1H, dd,J=14.5, 6.3 Hz), 3.27-3.43 (1H, m), 4.33-4.65 (2H, m), 4.90-5.15 (3H, m), 5.08 (2H, s), 6.52 (1H, d, J=7.3 Hz), 7.00 (1H, d, J=2.3 Hz), 7.03-7.28 (3H, m), 7.30-7.47 (5H, m), 7.59 (1H, dd, J=5.6, 1.7 Hz), 7.90 (1H, brs); MS: (LCMS) m/z 580 [M+H]⁺.

b) To a stirred solution of 4.33 g (7.47 mmol) of 2(S)-[2(S)-tert-butoxycarbonylamino-3-(1H-indol-3-yl)-propionylamino]-pentanedioic acid 5-benzyl ester 1-tert-butyl ester in 80 mL of ethyl acetate was added catalytic amount of palladium carbon. The mixture was stirred over night under hydrogen atmosphere at room temperature. The reaction was filtered to remove catalyst and solvent was removed under reduced pressure to give 2(S)-[2(S)-tert-butoxycarbonylamino-3-(1H-indol-3-yl)-propionylamino]-pentanedioic acid 1-tert-butyl ester as a white amorphous (3.70 g, quant). This product was used in the next reaction step without further purification. MS: (LCMS) m/z 490 [M+H]⁺.

c) To a stirred solution of 3.70 g (7.56 mmol) of 2(S)-[2(S)-tert-butoxycarbonylamino-3-(1H-indol-3-yl)-propionylamino]-pentanedioic acid 1-tert-butyl ester in 200 mL of dichloromethane was added 1.83 g(14.9 mmol) of 4-(dimethylamino)pyridine, 5.73 g (29.9 mmol) of 1-[3-(dimethylamino)propyl]-3-ethylcarbodiimide hydrochloride and 1.73 g (4.98 mmol) of camptotbecin. The mixture was stirred for 2 hours under nitrogen at room temperature. The reaction was quenched by addition of water, and organic layer was separated. The aqueous layer was extracted with dichloromethane. The combined organic layer was washed with 0.5 N hydrochloride solution, saturated sodium hydrogen carbonate solution, water and brine. The extract was dried over anhydrous magnesium sulfate and filtered. The solvent was removed under reduced pressure. The crude product was purified by medium pressure liquid chromatography with Lobar LiChroprep Si-60 Grobe C (eluent:ethyl acetate/dichloromethane=20/1) to give 2(S)-[2(S)-tert-Butoxycarbonylamino-3-(1H-indol-3-yl)-propionylamino]-pentanedioic acid 1-tert-butyl ester 5-(4(S)-ethyl-3,13-dioxo-3,4,12,13-tetrahydro-1H-2-oxa-6,12a-diaza-dibenzo[b,h]fluoren-4-yl)ester as a yellow amorphous (3.95 g, 97%). MS: (LCMS) m/z 820 [M+H]⁺.

d) To a stirred solution of 3.95 g (4.82 mmol) of 2(S)-[2(S)-tert-butoxycarbonylamino-3-(1H-indol-3-yl)-propionylamino]-pentanedioic acid 1-tert-butyl ester-α-5-(4(S)-ethyl-3,13-dioxo-3,4,12,13-tetrahydro-1H-2-oxa-6,12a-diaza-dibenzo[b,h]fluoren-4-yl)ester in 20 mL of ethyl acetate was added 40 mL of 1N hydrochloride acetic acid and 20 mL of trifluoroacetic acid. The mixture was stirred over night under nitrogen at room temperature. The reaction was added 800 mL of ethyl acetate, and then the precipitate was filtered to give 20-O—[(S)-tryptophyl-γ-(S)-glutamyl]-20-(S)-camptothecin hydrochloride as reddish solid (3.2 g, 95%).

¹H-NMR: (270 MHz, CD₃OD) δ1.03(3H, t, J=7.5 Hz), 2.02-2.39 (4H, m), 2.68-2.83(2H,m), 3.06-3.23(2H,m), 3.27-3.35(m), 3.38-3.50(2H,m), 4.17-4.33(1H, m) 4.42-4.57 (1H, m), 4.79-4.97(m), 5.20-5.38(2H, m), 5.55(2H, dd, J=38.6, 16.8 Hz), 6.97(1H, t, J=7.6 Hz), 7.11(1H, t, J=7.9 Hz), 7.19(1H, s), 7.36(2H, d, J=8.3 Hz), 7.58(1H, s), 7.65 (1H, d,J=7.9 Hz), 7.76(1H, t,J=6.9 Hz), 7.83-7.93(1H, m), 8.11(1H, d,J=8.6 Hz), 8.25(1H, d, J=8.3 Hz), 8.77(1H,s); MS: (LCMS) m/z 664 [M+H]⁺.

The following compounds in examples 25-49 were prepared from camptothecin or SN38 using a different dipeptide derivative of formula (VII) by the method similar to Example 24.

### Example 25

20-O—[(S)-valyl-γ-(S)-glutamyl]-20(S)-camptothecin hydrochloride was prepared from 2(S)-[(2(S)-tert-Butoxycarbonylamino-3-methyl-butyrylamino]-pentanedioic acid 1-tert-butyl ester

¹H-NMR: (270 MHz, CD₃OD) δ0.97-1.14(9H, m), 1.93-2.38 (6H, m), 2.63-2.92(2H,m), 3.27-3.34(m), 3.81(1H, d, J=5.3 Hz), 4.48-4.59 (1H, m), 4.80-4.97 (m), 5.38(2H, brs), 5.55(2H, dd, J=35.6, 17.2 Hz), 7.69(1H, s), 7.80(1H, t, J=8.3 Hz), 8.00(1H, td, J=8.6, 1.3 Hz), 8.18(1H, d, J=8.3 Hz), 8.33 (1H, d, J=8.6 Hz), 8.88(1H,s); MS: (LCMS) m/z 577 [M+H]⁺.

### Example 26

20-O—[(S)-phenylalanyl-γ-(S)-glutamyl]-20(S)-camptothecin hydrochloride was prepared from 2(S)-[(2(S)-tert-Butoxycarbonylamino-3-phenyl-propionylamino]-pentanedioic acid 1-tert-butyl ester.

¹H-NMR: (270 MHz, CD₃OD) δ0.99-1.10 (3H, m), 1.33-1.42 (1H, m), 1.97-2.42 (4H, m), 2.73 (1H, t, J=9.6 Hz), 2.93-3.12 (1H, m), 3.19-3.40 (m), 3.52-3.82 (2H, m), 4.02-4.26 (1H,m), 4.40-4.60 (1H, m), 4.71-4.91 (m), 5.28-5.39 (2H, m), 5.56(2H, dd, J=38.3, 17.2 Hz), 7.14-7.23 (1H, m), 7.25-7.43 (6H, m), 7.73 (1H, t, J=6.9 Hz), 7.88 (1H, t, J=8.6 Hz), 8.09 (1H, d, J=8.3 Hz), 8.13-8.22 (1H, m), 8.64(1H,d, J=8.3 Hz); MS: (LCMS) m/z 625[M+H]⁺.

### Example 27

20-O—[(S)-leucyl-γ-(S)-glutamyl]-20(S)-camptothecin hydrochloride Was Prepared From 2(S)-[2(S)-tert-Butoxycarbonylamino-4-methyl-pentanoylamino]-pentanedioic acid 1-tert-butyl ester.

¹H-NMR: (270 MHz, CD₃OD) δ0.89-1.12 (11H, m), 1.58-2.41 (8H, m), 2.60-2.99 (2H,m), 3.23-3.36(m), 3.51-3.79 (7H, m), 3.87 (1H, brs), 4.00-4.11 (1H, m), 4.47-4.63 (1H, m), 4.74-5.00 (m), 5.39 (2H, brs), 5.54(2H, dd, J=36.0, 17.5 Hz), 7.78-7.94 (2H, m), 7.98-8.11 (1H, m), 8.24 (1H, d, J=8.3 Hz), 8.42 (1H, d, J=8.6 Hz), 8.99(1H,s); MS: (LCMS) m/z 591[M+H]⁺.

### Example 28

20-O—[(R)-leucyl-γ-(S)-glutamyl]-20(S)-camptothecin hydrochloride Was Prepared From 2(S)-[2(R)-tert-Butoxycarbonylamino-4-methyl-pentanoylamino]-pentanedioic acid 1-tert-butyl ester

¹H-NMR: (270 MHz, CD₃OD) d 0.83-1.12 (10H, m), 1.60-1.82 (3H, m), 1.90-2.40(3H,m), 2.54-2.80 (2H, m), 3.17-3.40 (m), 3.51-3.80 (3H, m), 3.81-3.97 (1H, m), 4.43-4.60 (1H, m), 4.62-5.00 (m), 5.32(2H, brs), 5.54(2H, dd, J=37.3, 17.0 Hz), 7.38(1H, s), 7.71(1H, t, J=7.3 Hz), 7.87 (1H, t, J=7.8 Hz), 8.06 (1H, d, J=7.6 Hz), 8.16 (1H, d, J=8.4 Hz), 8.63(1H,s); MS: (LCMS) m/z 591[M+H]⁺.

### Example 29

20-O—[(R)-phenylalanyl-γ-(S)-glutamyl]-20(S)-camptothecin trifluoroacetic acid Was Prepared From 2(S)-[(2(R)-tert-Butoxycarbonylamino-3-phenyl-propionylamino]-pentanedioic acid 1-tert-butyl ester

¹H-NMR: (270 MHz, CD₃OD) γ1.05 (3H, t, J=7.6 Hz), 1.70-1.99 (1H, m), 2.02-2.47 (5H, m), 2.95-3.22 (2H, m), 3.23-3.41 (m), 4.09 (1H, t, J=7.9 Hz), 4.43 (1H, dd, J=9.2, 4.6 Hz), 4.70-4.90 (m), 5.29 (2H, d, J=3.3 Hz), 5.54(2H, dd, J=38.3, 17.2 Hz), 7.19-7.49 (6H, m), 7.72 (1H, td, J=6.9, 1.3 Hz), 7.88 (1H, td, J=6.9, 1.3 Hz), 8.08 (1H, d, J=7.3 Hz), 8.17 (1H, d, J=8.6 Hz), 8.62(1H,s); MS: (LCMS) m/z 625[M+H]⁺.

### Example 30

20-O—[(S)-tryptophyl-γ-(R)-glutamyl]-20(S)-camptothecin hydrochloride Was Prepared From 2(R)-[2(S)-tert-Butoxycarbonylamino-3-(1H-indol-3-yl)-propionylamino]-pentanedioic acid 1-tert-butyl ester

¹H-NMR: (270 MHz, CD₃OD) δ1.03 (3H, t, J=7.6 Hz), 1.69-1.98 (1H, m), 2.05-2.36 (5H, m), 3.14 (2H, d, J=36.6 Hz), 3.22-3.39 (m), 4.09 (1H, t, J=6.9 Hz), 4.33-4.46 (1H, m), 4.72-5.02 (m), 5.03 (2H, dd, J=42.2, 18.8 Hz), 5.53(2H, dd, J=45.5, 16.8 Hz), 6.80-6.98 (2H, m), 7.10 (1H, t, J=7.3 Hz), 7.29 (1H, d, J=7.9 Hz), 7.32-7.41(2H, m), 7.69 (1H, td, J=13.9, 6.9 Hz), 7.78-7.89 (1H, m), 8.00 (1H, d, J=7.9 Hz), 8.10 (1H, d, J=8.6 Hz), 8.44(1H,s); MS: (LCMS) m/z 664 [M+H]⁺.

### Example 31

20-O—[(R)-tryptophyl-γ-(R)-glutamyl]-20(S)-camptothecin hydrochloride Was Prepared From 2(R)-[2(R)-tert-Butoxycarbonylamino-3-(1H-indol-3-yl)-propionylamino]-pentanedioic acid 1-tert-butyl ester

¹H-NMR: (270 MHz, CD₃OD) δ1.03 (3H, t, J=7.3 Hz), 1.94-2.40 (6H, m), 2.73 (2H, t, J=7.3 Hz), 3.00-3.17 (1H, m), 3.19-3.48 (m), 3.53-4.78 (2H, m), 4.08-4.23 (1H, m), 4.45-4.58 (1H, m), 4.72-5.00 (m), 5.21 (2H, dd, J=40.9, 19.8 Hz), 5.52(2H, dd, J=40.9, 16.8 Hz), 6.94-7.19 (3H, m), 7.32-7.40 (2H, m), 7.60 (1H, d, J=7.9 Hz), 7.70(1H, td, J=8.3, 1.3 Hz), 7.87 (1H, td, J=8.6, 1.3 Hz), 8.08 (1H, d, J=7.3 Hz), 8.17 (1H, d,J=8.6 Hz), 8.59(1H, s);

MS: (LCMS) m/z 664 [M+H]⁺.

### Example 32

20-O—[(S)-phenylalanyl-γ-(R)-glutamyl]-20(S)-camptothecin hydrochloride Was Prepared From 2(R)-[(2(5)-tert-Butoxycarbonylamino-3-phenyl-propionylamino]-pentanedioic acid 1-tert-butyl ester

¹H-NMR: (270 MHz, CD₃OD) δ0.97 (3H, t, J=7.3 Hz), 1.60-2.38 (7H, m), 2.98(2H, d, J=7.9 Hz), 3.14-3.29 (m), 4.00 (1H, t, J=7.3 Hz), 4.28-4.39 (1H, m), 4.70-4.89 (m), 5.29 (2H, d, J=4.6 Hz), 5.54(2H, dd, J=40.3, 16.8 Hz), 7.02-7.32 (7H, m), 7.62 (1H, td J=7.3, 1.0 Hz), 7.77 (1H, td, J=8.6, 1.7 Hz), 7.99(1H, d, J=8.2 Hz), 8.05 (1H, d, J=8.6 Hz), 8.52(1H,s); MS: (LCMS) m/z 625[M+H]⁺.

### Example 33

20-O—[(S)-leucyl-γ-(R)-glutamyl]-20(S)-camptothecin hydrochloride Was Prepared From 2(R)-[2(S)-tert-Butoxycarbonylamino-4-methyl-pentanoylamino]-pentanedioic acid 1-tert-butyl ester

¹H-NMR: (270 MHz, CD₃OD) δ0.91-1.09(10H, m), 1.60-1.81 (2H, m), 1.94-2.41(3H,m), 2.70(2H, d, J=8.6 Hz), 3.23-3.38(m), 3.82-3.98(1H, m), 4.43-4.45 (1H,m) 4.79-4.97(m), 5.32(2H, brs), 5.54(2H, dd, J=38.9, 16.8 Hz), 7.40(1H, s), 7.71(1H, td, J=7.3, 1.3 Hz), 7.82-7.93(1H, m), 8.08(1H, d, J=7.3 Hz), 8.18 (1H, d, J=8.6 Hz), 8.64(1H,s); MS:(LCMS) m/z 591[M+H]⁺.

### Example 34

20-O—[(R)-tryptophyl-γ-(S)-glutamyl]-20(S)-camptothecin hydrochloride Was Prepared From 2(S)-[2(R)-tert-Butoxycarbonylamino-3-(1H-indol-3-yl)-propionylamino]-pentanedioic acid 1-tert-butyl ester

¹H-NMR: (270 MHz, CD₃OD) δ0.97-1.10 (3H, m), 1.68-2.48 (9H, m), 2.62-2.78(1H,m), 2.94-3.47(m), 3.93-4.36(3H, m), 4.46-4.58(1H, m), 4.80-4.98(m), 5.02-5.29(2H, m), 5.52(2H, dd, J=40.3, 17.2 Hz), 6.85(1H, s), 6.96-7.24(5H, m), 7.34(2H, s), 7.39(1H,s), 7.44(2H, t, J=7.6 Hz), 7.62(1H, d, J=7.9 Hz), 7.71 (2H, t, J=6.9 Hz), 7.78-7.90(2H, m), 8.00-8.18(4H, m), 8.49(1H, s), 8.58(1H,s); MS: (LCMS) m/z 664 [M+H]⁺.

### Example 35

20-O—[(R)-phenylalanyl-γ-(R)-glutamyl]-20(S)-camptothecin hydrochloride Was Prepared From 2(R)-[(2(R)-tert-Butoxycarbonylamino-3-phenyl-propionylamino]-pentanedioic acid 1-tert-butyl ester

¹H-NMR: (270 MHz, CD₃OD) δ1.03(3H, t, J=7.6 Hz), 1.90-2.38 (4H, m), 2.65-2.78(2H,m), 2.94-3.08(1H, m), 3.22-3.34(m), 4.10-4.22(1H, m), 4.48-4.57 (1H, m), 4.73-4.98(m), 5.30(2H, brs), 5.54(2H, dd, J=40.3, 16.8 Hz), 7.21-7.43(6H, m), 7.70(1H, t, J=7.9 Hz), 7.81-7.92(1H, m), 8.08(1H, d, J=8.2 Hz), 8.17 (1H, d, J=8.6 Hz), 8.62(1H,s);

MS: (LCMS) m/z 625[M+H]⁺.

### Example 36

20-O—[(R)-leucyl-γ-(R)-glutamyl]-20(S)-camptothecin hydrochloride Was Prepared From 2(R)-[2(R)-tert-Butoxycarbonylamino-4-methyl-pentanoylamino]-pentanedioic acid 1-tert-butyl ester

¹H-NMR: (270 MHz, CD₃OD) δ0.91-1.09(9H, m), 1.58-2.39 (8H, m), 2.68-2.80(2H,m), 3.25-3.34(m), 3.86-3.98(1H, m), 4.49-4.60 (1H, m), 4.79-4.97(m), 5.31(2H, brs), 5.54(2H, dd, J=38.9, 16.8 Hz), 7.40(1H, s), 7.68-7.75(1H, m), 7.83-7.93(1H, m), 8.08(1H, d, J=8.2 Hz), 8.18 (1H, d, J=8.3 Hz), 8.64(1H,s); MS: (LCMS) m/z 591[M+H]⁺.

### Example 37

7-ethyl-10-hydroxy-20-O—[(R)-tryptophyl-γ-(R)-homoglutamyl]-20(S)-camptothecin hydrochloride Was Prepared From 2(R)-[2(R)-tert-Butoxycarbonylamino-3-(1H-indol-3-yl)-propionylamino]-hexanedioic acid 1-tert-butyl ester

¹H-NMR: (270 MHz, CD₃OD) δ1.04(3H, t, J=7.6 Hz), 1.39(3H, t, J=7.6 Hz), 1.60-2.34 (6H, m), 2.53-2.70(2H, m), 3.02-3.49(m), 4.09-4.19(1H, m), 4.38-4.52(1H, m), 5.16-5.33(2H, m), 5.52(2H, dd, J=41.2, 17.3 Hz), 6.92-7.48(8H, m), 7.65(1H, d, J=8.1 Hz), 7.98-8.08(1H,m); MS: (LCMS) m/z [M+H]⁺.

### Example 38

7-ethyl-10-hydroxy-20-O—[(R)-tryptophyl-γ-(R)-glutamyl]-20(S)-camptothecin hydrochloride Was Prepared From 2(R)-[2(R)-tert-Butoxycarbonylamino-3-(1H-indol-3-yl)-propionylamino]-pentanedioic acid 1-tert-butyl ester

¹H-NMR: (270 MHz, CD₃OD) δ1.03(3H, t, J=7.3 Hz), 1.36(3H, t, J=7.6 Hz), 2.01-2.36 (4H, m), 2.73(2H, t, J=7.3 Hz), 3.02-3.18(3H, m), 3.26-3.44(m), 4.15(1H, dd, J=8.9, 5.4 Hz), 4.52(1H, dd, J=9.2, 4.6 Hz), 4.82-4.98(m), 5.09(2H, dd, J=44.6, 18.6 Hz), 5.52(2H, dd, J=44.0, 16.7 Hz), 6.99-7.15(4H, m), 7.29(1H,s), 7.34-7.43(3H, m), 7.60(1H, d, J=8.1 Hz), 8.00(1H,d, J=9.7 Hz); MS: (LCMS) m/z 708 [M+H]⁺.

### Example 39

7-ethyl-10-hydroxy-20-O—[(S)-phenylalanyl-γ-(R)-glutamyl]-20(S)-camptothecin hydrochloride Was Prepared From 2(R)-[2(S)-tert-butoxycarbonylamino-3-phenyl-propionylamino)]-pentanedioic acid 1-tert-butyl ester

¹H-NMR: (270 MHz, CD₃OD) δ1.05(3H, t, J=7.4 Hz), 1.38(3H, t, J=7.6 Hz), 1.86(1H, m), 2.05-2.40 (5H, m), 3.04(2H, br.d), 3.14(2H, br.q), 4.08(1H, t,J=7.4 Hz), 4.42(1H, m), 5.16(1H, d,J=18.8 Hz), 5.26(1H, d,J=18.8 Hz), 5.46(1H, d,J=16.8 Hz), 5.62(1H, d, J=16.8 Hz), 7.13(2H, m), 7.28(4H, m), 7.42(2H, m), 8.00(1H, d,J=7.3 Hz ); MS: (FABMS) m/z 669 [M+H]⁺.

### Example 40

7-ethyl-10-hydroxy-20-O—[(S)-phenylalanyl-β-(S)-aspartyl]-20(S)-camptothecin hydrochloride Was Prepared From 2(S)-[(2(S)-tert-butoxycarbonylamino-3-phenyl-propionylamino)-succinic acid 1-tert-butyl ester

¹H-NMR: (270 MHz, CD₃OD) δ1.04(3H, t, J=7.4 Hz), 1.43(3H, t, J=7.6 Hz), 2.22(2H, m), 2.97-3.35(6H, m), 4.15(1H, m), 4.83(1H, m), 5.38(2H, s), 5.50(1H, d, J=17.2 Hz), 5.63(1H, d, J=17.2 Hz), 7.30(5H, s), 7.57-7.68(3H, m), 8.23(1H, m); MS: (FABMS) m/z 655 [M+H]⁺.

### Example 41

7-etbyl-10-hydroxy-20-O—[(S)-leucyl-β-(S)-aspartyl]-20(S)-camptothecin hydrochloride Was Prepared From 2(S)-[(2(S)-tert-butoxycarbonylamino-4-methyl-pentanoylamino)-succinic acid 1-tert-butyl ester

¹H-NMR: (270 MHz, CD₃OD) δ0.94-1.08(9H, m), 1.42(3H, t, J=7.6 Hz), 1.61-1.83(3H, m), 2.12-2.31(2H, m), 3.18-3.35(4H, m), 3.88(1H, m), 4.85(1H, m), 5.38(2H, s), 5.50(1H, d, J=17.2 Hz), 5.63(1H, d, J=17.2 Hz), 7.50-7.61(3H, m), 8.16(1H, d, J=9.2 Hz );

MS: (FABMS) m/z 621 [M+H]⁺.

### Example 42

20-O—[(S)-tryptophyl-β-(R)-aspartyl]-20(S)-camptothecin hydrochloride Was Prepared From 2(R)-[(2(S)-tert-butoxycarbonylamino-3-(1H-indol-3-yl)-propionylamino]-succinic acid 1-tert-butyl ester

¹H-NMR: (270 MHz, DMSO-d6) δ0.87(3H, t, J=7.6 Hz), 2.18(2H, m), 2.70-3.03 (4H, m), 3.84(1H, m), 4.34(1H,d, J=19.2 Hz), 4.70(1H, m), 4.98(1H,d, J=19.2 Hz), 5.49(2H, s), 6.56(1H,t, J=7.5 Hz), 6.77(1H, s), 6.93(1H,t, J=7.2 Hz), 7.07(1H,d, J=7.9 Hz), 7.14(1H, s), 7.25(1H,d, J=7.9 Hz), 7.70(1H,t, J=7.0 Hz), 7.84(4H, m), 8.01(1H,d, J=7.6 Hz), 8.11(1H, d, J=8.6 Hz), 8.25(1H, s), 9.05(1H,d, J=8.6 Hz), 10.8(1H, s); MS: (FABMS) m/z 650 [M+H]⁺.

### Example 43

20-O—[(S)-phenylalanyl-β-(R)-aspartyl]-20(S)-camptothecin hydrochloride Was Prepared From 2(R)-[(2(S)-tert-butoxycarbonylamino-3-phenyl-propionylamino)-succinic acid 1-tert-butyl ester

¹H-NMR: (270 MHz, DMSO-d6) δ0.90(3H, t, J=7.3 Hz), 2.18(2H, m), 2.55-3.05(4H, m), 3.95(1H, m), 4.68(1H, m), 4.74(1H, d, J=19.3 Hz), 5.13(1H, d, J=19.3 Hz), 5.50(2H, s), 6.76(2H, d, J=7.3 Hz), 6.99(2H, t, J=7.5 Hz), 7.13(1H, t, J=7.6 Hz), 7.14(1H, s), 7.73(1H, t, J=7.6 Hz), 7.88(1H, t, J=7.3 Hz), 7.94(3H, m), 8.14(2H, br.t), 8.53(1H, s), 9.03(1H, d, J=8.6 Hz); MS: (FABMS) m/z 611 [M+H]⁺.

### Example 44

20-O—[(R)-phenylalanyl-β-(R)-aspartyl]-20(S)-camptothecin hydrochloride Was Prepared From 2(R)-[(2(R)-tert-butoxycarbonylamino-3-phenyl-propionylamino)-succinic acid 1-tert-butyl ester

¹H-NMR: (270 MHz, DMSO-d6) δ0.91(3H, t,J=7.5 Hz), 2.18(2H, m), 2.87-3.09(4H, m), 3.99(1H, m), 4.67(1H, m), 5.26(2H, br.s), 5.50(2H, br.s), 7.16-7.27(6H, m), 7.74(1H, br.t), 7.88(1H, br.t), 8.03(3H, m), 8.16(2H, br.t), 8.69(1H, s), 9.12(1H, d, J=7.9 Hz); MS: (FABMS) m/z 611 [M+H]⁺.

### Example 45

20-O—[(S)-phenylalanyl-β-(S)-aspartyl]-20(S)-camptothecin hydrochloride Was Prepared From 2(S)-[(2(S)-tert-butoxycarbonylamino-3-phenyl-propionylamino)-succinic acid 1-tert-butyl ester

¹H-NMR: (270 MHz, CD₃OD) δ1.04(3H, t, J=7.4 Hz), 2.13-2.32(2H, m), 2.96-3.37(4H, m), 4.13(1H, m), 4.85(1H, m), 5.36(2H, s), 5.49(1H, d, J=17.2 Hz), 5.62(1H, d, J=17.2 Hz), 7.30(5H, br.s), 7.50(1H, s), 7.76(1H, br.t), 7.93(1H, br.t), 8.14(1H, d, J=7.6 Hz ), 8.22(1H, d, J=8.6 Hz ), 8.76(1H, s ); MS: (FABMS) m/z 611 [M+H]⁺.

### Example 46

20-O—[(S)-leucyl-β-(R)-aspartyl]-20(S)-camptothecin hydrochloride Was Prepared From 2(R)-[(2(S)-tert-butoxycarbonylamino-4-methyl-pentanoylamino)-succinicacid 1-tert-butyl ester

¹H-NMR: (270 MHz, DMSO-d6) δ0.67(3H, d, J=7.4 Hz), 0.68(3H, d, J=7.4 Hz), 0.89(3H, t, J=7.6 Hz), 1.43-1.62(3H, m), 2.15(2H, m), 3.03(2H, m), 3.78(1H, m), 4.64(1H, m), 5.32(2H, br.s), 5.48(2H, br.s), 7.17(1H, s), 7.74(1H, br.t), 7.89(1H, br.t), 8.16(5H, m), 8.72(1H, s ), 8.95(1H, d, J=7.3 Hz); MS: (FABMS) m/z 577 [M+H]⁺.

### Example 47

20-O—(S)-valyl-β-(R)-aspartyl]-20(S)-camptothecin hydrochloride Was Prepared From 2(R)-[(2(S)-tert-butoxycarbonylamino-3-methyl-butyrylamino]-succinicacid 1-tert-butyl ester

¹H-NMR: (270 MHz, CD₃OD) δ0.66(3H, d, J=6.9 Hz), 0.70(3H, d, J=6.9 Hz), 1.03(3H, t, J=7.5 Hz), 1.98(1H, m), 2.22(2H, m), 3.22(2H, m), 3.64(1H, d, J=5.3 Hz), 4.85(1H, m), 5.38(2H, br.s), 5.48(1H, d, J=16.8 Hz), 5.61(1H, d, J=16.8 Hz), 7.47(1H,s), 7.77(1H, br.t), 7.94(1H, br.t), 8.13(1H, br.d), 8.22(1H, br.d), 8.75(1H, s); MS: (FABMS) m/z 563 [M+H]⁺.

### Example 48

7-ethyl-10-hydroxy-20-O—[(S)-cyclohexylalanyl-γ-(R)-glutamyl]-20(S)-camptothecin hydrochloride Was Prepared From 2(R)-[(2(S)-tert-butoxycarbonylamino-3-cyclohexyl-propionylamino)-pentanedioic acid 1-tert-butyl ester

¹H-NMR: (270 MHz, DMSO-d6) δ0.81-2.66(22H, m), 0.92(3H, t, J=7.3 Hz), 1.29(3H, t, J=7.6 Hz), 3.02-3.17 (2H, m), 3.76-3.88(1H, m), 4.27-4.39(1H, m), 5.30(1H, s), 5.49(1H, s), 7.00(1H, d, J=8.2 Hz), 7.43-7.47(2H, m), 8.03(1H, dd, J=3.0, 8.2 Hz), 8.22(2H, bs), 8.94(1H, d, J=4.9 Hz); MS: (FABMS) m/z 675 [M+H]⁺.

### Example 49

7-ethyl-10-hydroxy-20-O—[(S)-cyclohexylalanyl-γ-(S)-glutamyl]-20(S)-camptothecin hydrochloride Was Prepared From 2(S)-[(2(S)-tert-butoxycarbonylamino-3-cyclohexyl-propionylamino)-pentanedioic acid 1-tert-butyl ester

¹H-NMR: (270 MHz, DMSO-d6) δ0.77-2.85(22H, m), 0.92(3H, t, J=7.3 Hz), 1.29(3H, t, J=7.6 Hz), 3.01-3.17 (2H, m), 3.76-3.90(1H, m), 4.30-4.45(1H, m), 5.31(1H, s), 5.50(1H, s), 7.02(1H, d, J=4.6 Hz), 7.40-7.50(2H, m), 8.04(1H, d, J=9.6 Hz), 8.22(2H, bs), 8.78(1H, d, J=7.6 Hz); MS: (FABMS) m/z 675 [M+H]⁺.

The compounds in Example 49-1-Example 49-25 were prepared from (9S)-9-ethyl-9-hydroxy-1-pentyl-1H,12H-pyrano[3″,4″:6′,7′]indolizino[1′,2′:6,5]pyrido[4,3,2-de]quinazoline-10,13(9H,15H)-dione using a different dipeptide derivative of formula (VII) by the method similar to Example 24. The dipeptides are listed in the table below

| Example | Dipeptide Moiety |
|---|---|
| 49-1 | HCL.(L)Trp-(L)-γ-Glu- |
| 49-2 | HCL.(L)Cyclohexylalanyl-(D)-γ-Glu- |
| 49-3 | HCL.(L)Phe-(D)-γ-Glu- |
| 49-4 | HCL.(L)Leu-(D)-γ-Glu- |
| 49-5 | 2HCL.(L)Lys-(L)-γ-Glu- |
| 49-6 | HCL.(L)Val-(D)-γ-Glu- |
| 49-7 | 2HCL.(L)Orn-(L)-γ-Glu- |
| 49-8 | MsOH.(L)Leu-(D)-γ-Glu- |
| 49-9 | 2HCL.(D)Lys-(L)-γ-Glu- |
| 49-10 | HCL.(L)Phe-(L)-β-Asp- |
| 49-11 | HCL.(L)Cyclohexylalanyl-(D)-β-Asp- |
| 49-12 | HCL.(L)Cyclohexylalanyl-(L)-β-Asp- |
| 49-13 | HCL.(L)Trp-(L)-β-Asp- |
| 49-14 | 2HCL.(L)Orn-(D)-γ-Glu- |
| 49-15 | HCL.(L)Leu-(D)-β-Asp- |
| 49-16 | HCL.(L)Val-(D)-β-Asp- |
| 49-17 | HCL.(L)Lcu-(L)-β-Asp- |
| 49-18 | HCL.(L)Cyclohexylglycyl-(L)-γ-Glu- |
| 49-19 | HCL.(D)Cyclohexylalanyl-(L)-γ-Glu- |
| 49-20 | 2HCL.(L)Lys-(D)-γ-Glu- |
| 49-21 | HCL.(L)Trp-(D)-γ-Glu- |
| 49-22 | HCL.(L)Leu-(L)-γ-Glu- |
| 49-23 | HCL.Gly-(D)-γ-Glu- |
| 49-24 | HCL.(L)Ala-(D)-γ-Glu- |
| 49-25 | HCL.(L)Phe-(D)-γ-Glu- |

### Example 49-1

(9S)-9-ethyl-9-[(L)-tryptophyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

MS (FAB) m/z 774 (MH⁺); ¹H NMR (270 MHz, CD₃OD) δ0.98 (t, J=7.0 Hz, 3H), 1.03 (t, J=7.6 Hz, 3H), 1.39-2.32 (m, 10H), 2.71-3.47 (m, 4H), 4.10-4.23 (m, 2H), 4.35 (m, 2H), 5.28 (d, J=17.2 Hz, 1H), 5.38 (d, J=17.2 Hz, 1H), 5.48 (d, J=17.2 Hz, 1H), 5.63 (d, J=17.2 Hz, 1H), 6.83 (t, J=7.3 Hz, 1H), 7.05 (br.t, 1H), 7.11 (s, 1H), 7.34(d, J=7.9 Hz, 1H), 7.48 (d, J=7.3 Hz, 1H), 7.62 (d, J=7.9 Hz, 1H), 7.92 (t, J=7.6 Hz, 1H), 7.97(br.d, 1H), 8.00 (s, 1H), 8.27 (s, 1H).

### Example 49-2

(9S)-9-ethyl-9-[(L)-cyclohexylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

MS (FAB) m/z 741 (MH⁺); ¹H NMR (270 MHz, CD₃OD) δ0.99 (t, J=7.3 Hz, 3H), 1.04 (t, J=7.3 Hz, 3H), 1.00-2.36 (m, 23H), 2.70-2.85 (m, 2H), 2.95 (br.t, J=7.8 Hz, 2H), 4.01 (m, 1H), 4.24 (br.t, 2H), 4.42 (m, 1H), 5.48 (d, J=17.5 Hz, 1H), 5.51 (s, 2H), 5.63 (d, J=17.5 Hz, 1H), 7.54 (d, J=7.9 Hz, 1H), 7.83 (s, 1H), 7.95 (d, J=7.6 Hz, 1H), 8.06 (br.t, 1H), 8.36 (s, 1H).

### Example 49-3

(9S)-9-ethyl-9-[(L)-phenylalanyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

MS (FAB) m/z 735 (MH⁺); ¹H NMR (270 MHz, CD₃OD) δ0.99 (t, J=7.3 Hz, 3H), 1.06 (t, J=7.3 Hz, 3H), 1.39-1.60 (m, 4H), 1.86-2.31 (m, 6H), 2.34-2.74 (m, 2H), 3.05-3.25 (m, 2H), 4.15-4.25 (m, 3H), 4.42 (m, 1H), 5.46 (s, 2H), 5.48 (d, J=17.2 Hz, 1H), 5.62 (d, J=17.2 Hz, 1H), 7.20-7.31 (m, 5H), 7.50 (d, J=7.6 Hz, 1H), 7.82 (s, 1H), 7.90 (d, J=8.3 Hz, 1H), 8.00 (br.t, 1H), 8.32 (s, 1H).

### Example 49-4

(9S)-9-ethyl-9-[(L)-leucyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

MS (FAB) m/z 701 (MH⁺); ¹H NMR (270 MHz, CD₃OD) δ0.94-1.08 (m, 12H), 1.39-2.35 (m, 13H), 2.65-2.85 (m, 2H), 3.98 (m, 1H), 4.23 (br.t, 2H), 4.42 (m, 1H), 5.49 (d, J=17.5 Hz, 1H), 5.50 (s, 2H), 5.63 (d, J=17.5 Hz, 1H), 7.53 (d, J=7.9 Hz, 1H), 7.79 (s,1H), 7.92 (d, J=8.3 Hz, 1H), 8.06 (br.t, 1H), 8.35 (s, 1H).

### Example 49-5

(9S)-9-ethyl-9-[(L)-lysyl-(L)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione dihydrochloride

MS (FAB) m/z 716 (MH⁺); ¹H NMR (400 MHz, CD₃OD) δ0.99 (t, J=7.2 Hz, 3H), 1.04 (t, J=7.2 Hz, 3H), 1.46-2.30 (m, 16H), 2.76-2.90 (m, 2H), 2.95 (br.t, J=7.8 Hz, 2H), 4.07 (t, J=6.6 Hz, 1H), 4.22 (br.t, 2H), 4.55 (dd, J=10.0, 4.4 Hz, 1H), 5.49 (d, J=16.8 Hz, 1H), 5.50 (s, 2H), 5.63 (d, J=16.8 Hz, 1H), 7.51 (d, J=8.0 Hz, 1H), 7.81 (s,1H), 7.93 (d, J=8.0 Hz, 1H), 8.06 (t, J=8.0 Hz, 1H), 8.30 (s, 1H).

### Example 49-6

(9S)-9-ethyl-9-[(L)-valyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

MS (FAB) m/z 687 (MH⁺); ¹H NMR (270 MHz, CD₃OD) δ0.97-1.11 (m, 12H), 1.40-1.59 (m, 4H), 1.88-2.33 (m, 7H), 2.66-2.89 (m, 2H), 3.80 (d, J=5.6 Hz, 1H), 4.23 (br.t, 2H), 4.43 (dd, J=9.2, 4.6 Hz, 1H), 5.49 (d, J=17.5 Hz, 1H), 5.51 (s, 2H), 5.63 (d, J=17.5 Hz, 1H), 7.54 (d, J=7.6 Hz, 1H), 7.80 (s, IH), 7.93 (d, J=7.6 Hz, 1H), 8.08 (br.t, 1H), 8.35 (s, 1H).

### Example 49-7

(9S)-9-ethyl-9-[(L)-ornithyl-(L)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione dihydrochloride

MS (FAB) m/z 702 (MH⁺); ¹H NMR (270 MHz, CD₃OD) δ0.99 (t, J=7.3 Hz, 3H), 1.04 (t, J=7.3 Hz, 3H), 1.40-2.38 (m, 14H), 2.68-3.00 (m, 2H), 3.01 (br.t, 2H), 4.11 (m, 1H), 4.24 (br.t, 2H), 4.57 (m, 1H), 5.48 (d, J=17.2 Hz, 1H), 5.51 (s, 2H), 5.64 (d, J=17.2 Hz, 1H), 7.57 (d, J=7.3 Hz, 1H), 7.93 (s, 1H), 8.01 (d, J=8.3 Hz, 1H), 8.11 (br.t, 1H), 8.33 (s, 1H).

### Example 49-8

(9S)-9-ethyl-9-[(L)-leucyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15 H)-dione methanesulfonic acid salt

S (FAB) m/z 701 (MH⁺); ¹H NMR (270 MHz, CD₃OD) δ0.95-1.08 (m, 12H), 1.39-2.35 (m, 13H), 2.66-2.75 (m, 2H), 2.72 (s, 9H), 3.96 (m, 1H), 4.25 (br.t, 2H), 4.45 (m, 1H), 5.49 (d, J=17.5 Hz, 1H), 5.51 (s, 2H), 5.63 (d, J=17.5 Hz, 1H), 7.52 (s, 1H), 7.54 (d, J=7.9 Hz, 1H), 7.88 (d, J=8.6 Hz, 1H), 8.09 (br.t, 1H), 8.38 (s, 1H).

### Example 49-9

(9S)-9-ethyl-9-[(D)-lysyl-(L)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione dihydrochloride

MS (FAB) m/z 716 (MH⁺); ¹H NMR (400 MHz, CD₃OD) δ0.95-1.07 (m, 6H), 1.42-2.30 (m, 16H), 2.64-2.95 (m, 2H), 2.99 (br.t, 2H), 4.02 (br.t, 1H), 4.26 (m, 2H), 4.39 (m, 1H), 5.51 (d, J=17.2 Hz, 1H), 5.52 (s, 2H), 5.63 (d, J=17.2 Hz, 1H), 7.55 (d, J=7.2 Hz, 1H), 7.96 (s, 1H), 7.99 (br.d, 1H), 8.09 (br.t, 1H), 8.33 (s, 1H).

### Example 49-10

(9S)-9-ethyl-9-[(L)-phenylalanyl-(L)-β-aspartyloxy]1-pentyl-1H,12H-pyrano[3″,4″:6′,7′]indolizino[1′,2′:6,5]pyrido[4,3,2-de]quinazoline-10,13(9H,15H)-dione hydrochloride

MS (FAB) m/z 721 (MH⁺); ¹H NMR (270 MHz, CD₃OD) δ0.99 (t, J=7.3 Hz, 3H), 1.04 (t, J=7.3 Hz, 3H), 1.42-2.30 (m, 8H), 2.98-3.37 (m, 4H), 4.17 (m, 1H), 4.24 (br.t, 2H), 4.82 (m, 1H), 5.50 (s, 2H), 5.51 (d, J=17.2 Hz, 1H), 5.62 (d, J=17.2 Hz, 1H), 7.31 (br.s, 5H), 7.53 (d, J=7.3 Hz, 1H), 7.68 (s, 1H), 7.83 (d, J=7.6 Hz, 1H), 8.08 (br.t, 1H), 8.36 (s, 1H).

### Example 49-11

(9S)-9-ethyl-9-[(L)-cyclohexylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

MS (FAB) m/z 727 (MH⁺); ¹H NMR (270 MHz, CD₃OD) δ0.98 (t, J=7.3 Hz, 3H), 1.04 (t, J=7.3 Hz, 3H), 0.80-2.27 (m, 21H), 3.23 (br.d, J=6.0 Hz, 2H), 3.98 (m, 1H), 4.24 (br.t, 2H), 4.85 (m, 1H), 5.50 (d, J=17.2 Hz, 1H), 5.52 (s, 2H), 5.63 (d, J=17.2 Hz, 1H), 7.54 (d, J=7.6 Hz, 1H), 7.72 (s, 1H), 7.87 (d, J=7.6 Hz, 1H), 8.09 (t, J=7.6 Hz, 1H), 8.36 (s, 1H).

### Example 49-12

(9S)-9-ethyl-9-[(L)-cyclohexylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

MS (FAB) m/z 727 (MH⁺); ¹H NMR (270 MHz, CD₃OD) δ0.98 (t, J=7.3 Hz, 3H), 1.04 (t, J=7.3 Hz, 3H), 0.90-2.30 (m, 21H), 3.12-3.34 (m, 2H), 3.93 (m, 1H), 4.22 (br.t, 2H), 4.83 (m, 1H), 5.50 (s, 2H), 5.51 (d, J=17.5 Hz, 1H), 5.63 (d, J=17.5 Hz, 1H), 7.47 (br.d, 1H), 7.65 (br.s, 1H), 7.82 (br.d, 1H), 8.03 (br.t, 1H), 8.30 (s, 1H).

### Example 49-13

(9S)-9-ethyl-9-[(L)-tryptophyl-(L)-β-aspartyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

MS (FAB) m/z 760 (MH⁺); ¹H NMR (270 MHz, CD₃OD) δ0.98 (t, J=6.9 Hz, 3H), 1.00 (t, J=7.3 Hz, 3H), 1.39-1.60 (m, 4H), 1.83-1.97 (m, 2H), 2.13-2.30 (m, 2H), 3.05-3.40 (m, 4H), 4.12 (m, 2H), 4.29 (m, 1H), 4.79 (m, 1H), 5.27 (d, J=17.8 Hz, 1H), 5.36 (d, J=17.8 Hz, 1H), 5.51 (d, J=17.5 Hz, 1H), 5.63 (d, J=17.5 Hz, 1H), 6.86 (br.t, 1H), 7.06 (br.t, 1H), 7.10 (s, 1H), 7.29 (d, J=8.3 Hz, 1H), 7.43 (d, J=7.9 Hz, 1H), 7.53 (d, J=7.3 Hz, 1H), 7.84 (s, 1H), 7.85 (d, J=8.3 Hz, 1H), 8.06 (br.t, 1H), 8.30 (s, 1H).

### Example 49-14

(9S)-9-ethyl-9-[(L)-ornithyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione dihydrochloride

MS (LCMS) m/z 702 (MH⁺); ¹H NMR (400 MHz, CD₃OD) δ0.98 (t, J=7.0 Hz, 3H), 1.04 (t, J=7.4 Hz, 3H), 1.46-2.21 (m, 14H), 2.77-2.81 (m, 2H), 3.01 (br.t, J=7.4 Hz, 2H), 4.03 (t, J=6.6 Hz, 1H), 4.24 (m, 2H), 4.44 (m, 1H), 5.50 (d, J=16.8 Hz, 1H), 5.51 (s, 2H), 5.63 (d, J=16.8 Hz, 1H), 7.54 (d, J=8.0 Hz, 1H), 7.91 (s, 1H), 7.95 (d, J=8.0 Hz, 1H), 8.09 (t, J=8.0 Hz, 1H), 8.34 (s, 1H).

### Example 49-15

(9S)-9-ethyl-9-[(L)-leucyl-(D)-β-aspartyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

¹H NMR (270 MHz) δ (CD₃OD) 0.80-0.91 (m, 6H), 0.91-1.06 (m, 6H), 1.37-1.80 (m, 7H), 2.83-2.01 (m, 2H), 2.07-2.30 (m, 2H), 3.15-3.40 (m, 2H), 3.88-4.00 (m, 1H), 4.25 (br.t, 2H), 4.74-4.93 (m, 1H), 5.50 (d, J=17.2 Hz, 1H), 5.52 (s, 2H), 5.62 (d, J=17.2 Hz, 1H), 7.55 (d, J=7.9 Hz, 1H), 7.75 (s, 1H), 7.88 (d, J=8.6 Hz, 1H), 8.10 (br.t, 1H), 8.37 (s, 1H); MS (FAB) m/z 687 (MH⁺).

### Example 49-16

(9S)-9-ethyl-9-[(L)-valyl-(D)-β-aspartyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

¹H NMR (270 MHz) δ (CD₃OD) 0.86 (d, J=6.9 Hz, 3H), 0.88 (d, J=6.9 Hz, 3H), 0.94-1.08 (m, 6H), 1.40-1.61 (m, 4H), 1.89-2.05 (m, 2H), 2.05-2.28 (m, 3H), 3.13-3.38 (m, 2H), 3.77 (d, J=5.3 Hz, 1H), 4.25 (br.t, 2H), 4.77-4.91 (m, 1H), 5.49 (d, J=17.2 Hz, 1H), 5.53 (s, 2H), 5.62 (d, J=17.2 Hz, 1H), 7.55 (d, J=7.3 Hz, 1H), 7.79 (s, 1H), 7.91 (d, J=7.6 Hz, 1H), 8.10 (br.t, 1H), 8.37 (s, 1H); MS (FAB) m/z 673 (MH⁺).

### Example 49-17

(9S)-9-ethyl-9-[(L)-leucyl-(L)-β-aspartyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

¹H NMR (270 MHz) δ (CD₃OD) 0.79-1.00 (m, 12H), 1.30-1.51 (m, 4H), 1.30-1.75 (m, 3H), 1.75-1.92 (m, 2H), 2.00-2.21 (m, 2H), 3.00-3.30 (m, 2H), 3.76-3.85 (m, 2H), 3.76-3.85 (m, 1H), 4.09-4.20 (m, 2H), 4.68-4.85 (m, 1H), 5.41 (d, J=17.3 Hz, 1H) 5.43 (s, 2H), 5.57 (d, J=17.3 Hz, 1H), 7.43 (d, J=7.6 Hz, 1H), 7.65 (s, 1H), 7.76 (d, J=8.2 Hz, 1H), 7.99 (br.t, 1H), 8.27 (s, 1H); MS (FAB) m/z 687 (MH⁺).

### Example 49-18

(9S)-9-ethyl-9-[(L)-cyclohexylglycyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

¹H NMR (270 MHz) δ (CD₃OD) 0.92-1.07 (m, 6H), 1.7.-2.30 (m, 21H), 2.62-3.09 (m, 2H), 3.87 (d, J=5.6 Hz, 1H), 4.24 (br.t, 2H), 4.40-4.51 (m, 1H), 5.48 (d, J=17.2 Hz, 1H), 5.51 (s, 2H), 5.63 (d, J=17.2 Hz, 1H), 7.53 (d, J=7.6 Hz, 1H), 7.88 (s, 1H), 7.97 (d, J=8.0 Hz, 1H), 8.06 (br.t, 1H), 8.32 (s, 1H); MS (FAB) m/z 727 (MH⁺).

### Example 49-19

(9S)-9-ethyl-9-[(D)-cyclohexylalanyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

¹H NMR (270 MHz) δ (CD₃OD) 0.93-1.08 (m, 6H), 1.15-2.36 (m, 23H), 2.60-2.94 (m, 2H), 3.99 (br.t, 1H), 4.22 (br.t, 2H), 4.37-4.45 (m, 1H), 5.49 (d, J=17.3 Hz, 1H), 5.50 (s, 2H), 5.62 (d, J=17.3 Hz, 1H), 7.51 (d, J=7.9 Hz, 1H), 7.80 (s, 1H), 7.91 (d, J=8.2 Hz, 1H), 8.05 (br.t, 1H), 8.32 (s, 1H); MS (FAB) m/z 741 (MH⁺).

### Example 49-20

(9S)-9-ethyl-9-[(L)-lysyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione dihydrochloride

¹H NMR (270 MHz) δ (CD₃OD) 0.92-1.10 (m, 6H), 1.40-1.62 (m, 6H), 1.62-1.80 (m, 2H), 1.83-2.41 (m, 8H), 2.70-2.82 (m, 2H), 2.89-3.00 (m, 2H), 3.93-4.03 (m, 1H), 4.16-4.31 (m, 2H), 4.40-4.50 (m, 1H), 5.30 (d, J=17.2 Hz, 1H), 5.53 (s, 2H), 5.63 (d, J=17.2 Hz, 1H), 7.54 (d, J=7.6 Hz, 1H), 7.87 (s, 1H), 7.94 (d, J=8.2 Hz, 1H), 8.10 (brt.t, 1H), 8.35 (s, 1H); MS (FAB) m/z 716 (MH⁺).

### Example 49-21

(9S)-9-ethyl-9-[(L)-tryptophyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

¹H NMR (270 MHz, CD₃OD) δ0.99 (t, J=7.3 Hz, 3H), 1.04 (t, J=7.3 Hz, 3H), 1.40-2.39 (m, 9H), 2.19 (t, J=7.3 Hz, 1H), 2.58-2.74 (m, 1H), 2.91-3.05 (m, 1H), 3.10 (dd, J=15.2, 5.6 Hz, 1H), 3.40 (dd, J=15.2, 5.6 Hz, 1H), 3.95-4.15 (m, 2H), 4.21 (br.t, J=5.6 Hz, 1H), 4.58-4.69 (m, 1H), 5.00 (d, J=8.3 Hz, 1H), 5.20 (d, J=8.3 Hz, 1H), 5.48 (d, J=7.3 Hz, 1H), 5.67 (d, J=7.3 Hz, 1H), 6.59 (t, J=7.3 Hz, 1H), 6.66 (s, 1H), 6.94-7.03 (m, 2H), 7.25 (d, J=7.9 Hz, 1H), 7.45 (d, J=7.6 Hz, 1H), 7.87 (d, J=7.6 Hz, 1H), 7.92 (s, 1H), 7.97 (t, J=7.6 Hz, 1H), 8.18 (s, 1H); MS (FAB) m/z 774 (MH⁺).

### Example 49-22

(9S)-9-ethyl-9-[(L)-leucyl-(L)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

¹H NMR (270 MHz) δ (CD₃OD) 1.01-1.06 (m, 12H), 1.39-2.32 (m, 13H), 2.67-2.79 (m, 1H), 2.89-2.98 (m, 1H), 4.08 (br.t, J=5.6 Hz, 1H), 4.23 (br.t, 2H), 4.50 (dd, J=9.6, 4.6 Hz, 1H), 5.49 (d, J=17.2 Hz, 1H), 5.51 (s, 2H), 5.63 (d, J=17.2 Hz, 1H), 7.52 (dd, J=7.6, 1.0 Hz, 1H), 7.88 (s, 1H), 7.96 (dd, J=8.2, 1.0 Hz, 1H), 8.05 (br.t, 1H), 8.30 (s, 1H);

MS (ES) m/z 701(MH⁺).

### Example 49-23

(9S)-9-ethyl-9-[glycyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

¹H NMR (270 MHz) δ (CD₃OD) 0.95-1.05 (m, 6H), 1.39-2.30 (m, 10H), 2.62-2.89 (m, 2H), 3.48 (d, J=16.0 Hz, 1H), 3.86 (d, J=16.0 Hz, 1H), 4.23 (br.t, 7.7 Hz, 2H), 4.51 (dd, J=9.4, 4.5 Hz, 1H), 5.48 (d, J=17.2 Hz, 1H), 5.50 (s, 2H), 5.62 (d, J=17.2 Hz, 1H), 7.51 (d, J=7.9 Hz, 1H), 7.77 (s, 1H), 7.91 (d, J=7.6 Hz, 1H), 8.06 (br.t, 1H), 8.31 (s, 1H); MS (ES) m/z 645 (MH⁺).

### Example 49-24

(9S)-9-ethyl-9-[(L)-alanyl-(D)-γ-glutamyloxy]-1-pentyl-1H,12H-pyrano[3″,4″:6′,7′]-indolizino[1′,2′:6,5]pyrido[4,3,2-de]quinazoline-10,13(9H,15H)-dione hydrochloride

¹H NMR (270 MHz) δ (CD₃OD) 0.98 (t, J=7.3 Hz, 3H), 1.03 (t, J=7.3 Hz, 3H), 1.56 (d, J=6.9 Hz, 3H), 1.39-2.34 (m, 10H), 2.64-2.85 (m, 2H), 4.03 (q, J=6.9 Hz, 1H), 4.23 (br.t, J=7.6 Hz, 2H), 4.44 (dd, J=9.4, 4.8 Hz, 1H), 5.48 (d, J=17.2 Hz, 1H), 5.50 (s, 2H), 5.62 (d, J=17.2 Hz, 1H), 7.51 (d, J=7.9 Hz, 1H), 7.76 (s, 1H), 7.89 (d, J=8.2 Hz, 1H), 8.06 (br.t, 1H), 8.32 (s, 1H); MS (ES) m/z 659 (MH⁺).

### Example 49-25

(9S)-9-ethyl-9-[(L)-phenylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride

¹H NMR (270 MHz) δ (CD₃OD) 1.01 (t, J=7.3 Hz, 3H), 1.04 (t, J=7.3 Hz, 3H), 1.40-2.25 (m, 7H), 2.62 (dd, J=14.5, 9.9 Hz, 1H), 3.02 (dd, J=14.5, 4.3 Hz, 1H), 3.33-3.47 (m, 2H), 4.10-4.20 (m, 3H), 4.93-5.01 (m, 2H), 5.12 (d, J=18.5 Hz, 1H), 5.32 (d, J=18.5 Hz, 1H), 5.48 (d, J=17.2 Hz, 1H), 5.64 (d, J=17.2 Hz, 1H), 6.81-6.88 (m, 4H), 7.01 (m, 1H), 7.53 (d, J=7.6 Hz, 1H), 7.91 (s, 1H), 7.93 (br.d, 1H), 8.09 (br.t, 1H), 8.24 (s, 1H);

MS (FAB) m/z 721 (MH⁺).

### Example 50

Tumor-Specific Activation and Action of TTCs

50.1 Generation of Cells that Constitutively Express a High Level of Microsomal Dipeptidase

Among the enzymes that were selected according to the results of the oligonucleotide arrays, the full length cDNAs for microsomal dipeptidase (MDP) (GenBank Accession No. J05257. Adachi, H., et al. Primary structure of human microsomal dipeptidase deduced from molecular cloning. J. Biol. Chem, 265, 3992-3995 (1990)) were cloned at the HindIII site of pRC/CMV vector (Invitrogen, San Diego, USA, Catalog No. V750-20) and transfected into a human tumor cell line HCT116 (ATCC Number, CCL-247) that expressed only a low level of the microsomal dipeptidase mRNA. pRC/CMV vector without any cDNA was also transfected to the same cell line to generate a control cell line. Transfection of the DNA was carried out by using TransIT-LT2 (PanVera, Madison, USA, Calatog No. MIR2320) according to the manufacturer's instruction. The resulting transfectants were cultivated in MaCoy5A medium (Sigma, St Louis, USA, Catalog No. M8403) supplemented with 10% (v/v) fetal bovine serum and 1 mg/ml of geneticin disulfate (Wako, Osaka, Japan, Catalog No. 535-24624). The cells that grew in the presence of 1 mg/ml G418 were collected and cultivated in MaCoy5A medium.

To determine the microsomal dipeptidase activities in the cells, subconfluent monolayer cultures of HCT116 carring pRC/CMV, HCT116 bearing pRC/CMV-MDP (hereafter referred to as HCTS5) were washed with phosphate-buffered saline (PBS), harvested with cell scraper, suspended in PBS, and harvested by low speed centrifugation at 1000×g for 5 min. The cell pellets were suspended in PBS and lyseed by sonication with Polytron (5 sec. at maximum speed). By using the same method, the cell extracts of the granulocyte progenitors were also prepared from the CD34-positive mononuclear cells originated from the human umbilical cord blood. The floating granulocyte progenitors cultured on a confluent monolalyer of MS5 in the presence of 50 ng/ml Flt3 ligand, 100 ng/ml SCF, and 50 ng/ml TPO for 5 days were collected, washed with PBS, suspended in PBS, and lysed by homogenization with polytron. After the cell debris was removed by the centrifugation at 15,000×g for 15 min, the supernatants were used for the experiments.

After protein concentrations of the cell extracts were determined with DC protein assay kit (Bio-Rad, Hercules, USA, Catalog No.500-0116) according to the manufacture's instruction, the microsomal dipeptidase activities were also determined according to the method of Watanabe et. al. (Watanabe, T. et. al., Biochim. Biophys. Acta. 1298, 109-118 ((1996)). The cell extracts were incubated at 37° C. for 30 min in a 100 μl of reaction mixture containing 25 mM Tris-HCl (pH8.0), 10 μM ZnCl₂, 10 mM glycine-(D)-alanine, 20 μM FAD, 3.75 unit/ml D-amino-acid oxidase (Roche Diagnostics, Mannheim, Germany, Catalog No. 102 784). The reaction was terminated by adding 40 μl of 25% (w/v) trichloroacetic acid. After a centrifugation at 10,000×g for 5 min, a 100 μl of the supernatant was mixed with 20 μl of 0.1% (w/v) 2,4-dinitrophenylhydrazine that was dissolved in 2M HCl, and incubated at 37° C. for 15 min. Thereafter, the solution was mixed with 3.75M of NaOH and further incubated at room temperature for 10 min. The amounts of (D)-alanine produced from glycine-(D)-alanine by the cell extracts were calculated from the absorbance at 445 nm and also from the standard (D)-alanine.

The enzyme activities of one of the clones HCTS5 that carried the pRC/CMV-MDP exhibited a high level of microsomal dipeptidase activities (430 nmole (D)-alanine produced per minute per mg protein) as compared to those of the vector-transfected HCT116 (less than 1 nmole (D)-alanine produced per minute per mg protein) and granulocyte progenitors (less than 1 nmole (D)-alanine produced per minute per mg protein).

50.2 Growth Inhibitory Activities of TTCs, which is Dependent on Microsomal Didpetidase

By using 96-multi well plates, approximately 2×10³ cells per well of the HCT116 bearing pRC/CMV and HCT116S5 carrying pRC/CMV-MDP were cultured in 200 μl of McCoy5A medium supplemented with 10% FBS in the presence or absence of the indicated concentrations of the drugs at 37° C. under 5% CO₂ in humidified air. The cells were exposed to drugs for 24 hr (taxol, camptothecins and their prodrugs) or 96 hr (DMDC and its compounds). When the drug exposure time to the cells was 24 hr, culture medium containing drugs were removed, and the cells were washed and suspended in the fresh medium without drugs and further cultured at 37° C. under 5% CO₂ in humidified air for the indicated days. Then, 10 μl of WST-8 (Cell Counting Kit-8, Wako, Osaka, Japan, Catalog No.343-07623) were added to the cultures, and the cells were further incubated at 37° C. for 1 or 2 hr. Cell growth inhibition was calculated as IC₅₀ values according to optical densities at 450 nm and at 655 nm. To examine the effects of drugs on granulocyte progenitors, the granulocyte progenitors, which were expanded on a monolayer of MS-5 for 7 days, were collected and washed with RPMI1640 medium. Approximately 5000 cells were suspended in 200 μl of RPMI medium supplemented with 10% FBS and 50 ng/ml of G-CSF in the presence or absence of drugs and cultured in the presence of drugs for 24 hr (taxol, camptothecins and their prodrugs) or 7 days (DMDC and its prodrug) at 37° C. under 5% CO₂ in humidified air. When the exposure time of drugs to the cells was 24 hr, the drugs were removed at 24 hr after addition of drugs by washing the cells with the above medium, and the cells were further cultured for 6 days in the same medium without drugs. Therafter, 20 μl of WST-1 (Roche Diagnostics, Mannheim, Germany, Catalog No. 1644807) were added to the cultures, and the cells were further incubated at 37° C. for 6 hr. Cell growth inhibition was calculated as IC₅₀ values according to optical densities at 450 nm and at 655 nm. Growth inhibition by paclitaxcel, canptotbecin or DMDC was not significantly different among HCT116, HCT116/S5 and granulocyte progenitor cells. However, their compounds showed stronger anti-proliferative activity on HCT116/S5 that expressed higher microsomal dipeptidase activities than HCT116 and granulocyte progenitor cells that had very little microsomal dipaptidase activity (see the biological activities of examples).

### Example A

Tablets containing the following ingredients can be manufactured in a conventional manner:

| Ingredients | Per tablet | |
|---|---|---|
| Compound of example 4 | 10.0-300.0 | mg |
| Lactose | 125.0 | mg |
| Maize starch | 75.0 | mg |
| Talc | 4.0 | mg |
| Magnesium stearate | 1.0 | mg |

### Example B

Capsules containing the following ingredients can be manufactured in a conventional manner:

| Ingredients | Per capsule |
|---|---|
| Compound of example 4 | 100.0 mg |
| Lactose | 150.0 mg |
| Maize starch | 20.0 mg |
| Talc | 5.0 mg |

### Example C

Injection solutions can have the following composition:

| | |
|---|---|
| Compound of example 4 | 10.0 mg |
| Sodium chloride | q.s mg |
| Water for injection solutions | ad 2.0 ml |

1. A method for identifying an enzyme to design anti-cancer compounds that is selectively converted to active substances in tumors, said method comprises comparing the expression levels of genes or proteins in human tissue or cells from normal and tumor origin, and selecting an enzyme of which mRNA or protein levels in tumor tissue are higher by more than two-fold as compared to normal cells or tissue.

2. The method according to claim 1, wherein the enzyme is identified by analyses of DNA microarray, polymerase chain reaction, northern blotting and in situ hybridization, differential displays, RNase protection assay, protein arrays, western blotting, two dimensional gel electrophoresis or enzyme-linked immumosorbent assay.

3. The method according to claim 2, wherein the enzyme is identified by analysis of DNA microarray or polymerase chain reaction.

4. The method according to claim 1, wherein the normal cells or tissue are from hematopoietic progenitors derived from bone marrow or umbilical cord blood, intestine, or skin.

5. The method according to claim 1, wherein the human tissue or cells from tumor origin is from brain, lung, esophagus, breast, stomach, pancreas, liver, colon, rectum, kidney, ovary, uterus, bladder, prostate, skin, and blood.

6. A method of identifying anti-cancer compounds that are convertible to active substances selectively in tumors, said method comprises
(a) generating an enzyme by cell expression in which the protein levels in tumor tissue are higher by more than two-fold as compared to normal cells or tissue; and
(b) determining growth inhibitory activities of said anti-cancer compounds.

7. The method of claim 6, wherein said enzyme is microsomal dipeptidase, arylsulfatase A, pyrroline 5′-carboxyreductase, dehydrodiol dehydrogenase, carbonylreductase, lysyl hydroxylase, prolidase, dihydropyrimidinase, glutamine:fructose-6-phosphate amidotransferase, UDP-galactose ceramide galactosyl transferase, lysyl oxidase, enolase, glucose-6-phosphate dehydrogenase, stearoyl-coenzyme A desaturase, epoxide hydrolase or aldolase C.

8. An anti-cancer compound of formula (I),
X—Y-Q (I)
wherein X is a moiety that generates an active anti-cancer substance (Q-Y—H) selective to tumors by an enzyme; Q-Y— is a radical derived from the active anti-cancer substance (Q-Y—H) in which Y is —O—, —S— or —N—, or
a pharmaceutically acceptable salt thereof.

9. The compound of claim 8 wherein said enzymes is microsomal dipeptidase, arylsulfatase A, pyrroline 5′-carboxyreductase, dehydrodiol dehydrogenase, carbonylreductase, lysyl hydroxylase, prolidase, dihydropyrimidinase, glutamine:fructose-6-phosphate amidotransferase, UDP-galactose ceramide galactosyl transferase, lysyl oxidase, enolase, glucose-6-phosphate dehydrogenase, stearoyl-coenzyme A desaturase, epoxide hydrolase or aldolase C.

10. The compound of claim 8, wherein the radical (Q-Y—) of an active anti-cancer substance (Q-Y—H) is taxans, camptothecins, anti-cancer nucleosides, dolastatins, anthracyclins, farnesyltransferase inhibitors or EGF receptor tyrosine kinase inhibitors.

11. The compound of claim 10, wherein the active anti-cancer substance (Q-Y—H) is a taxan selected from the group consisting of
a) [2aR-[2aα,4β,4aβ,6β,9α(αR*, βS*),11α,12α,12aα,12bα]]-β-(benzoylamino)-α-hydroxybenzenepropanoic acid 6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca[3,4]benz[1,2-b]oxet-9-yl ester,
b) [2aR-[2aα,4β,4aα, 6β,9α(αR*,βS*,11α, 12α, 12aα, 12bα)]-β[[(1,1-dimethylethoxy)carbonyl]amino]-α-hydroxybenzenepropanoic acid 12b-(acetyloxy)-12-(benzoyloxy)-2a,3,4,4a,5,6,9,10,11,12,12a,12b-dodecahydro-4,6,11-trihydroxy-4a,8,13,13-tetramethyl-5-oxo-7,11-methano-1H-cyclodeca[3,4]benz[1,2-b]oxet-9-yl ester,
c) (2R,3S)-3-[[(1,1-dimethylethoxy)carbonyl]amino]-2-hydroxy-5-methyl-4-hexenoic acid (3aS,4R,7R,8aS,9S,10aR,12aS,12bR,13S,13aS)-7,12a-bis(acetyloxy)-13-(benzyloxy)-3a,4,7,8,8a,9,10,10a,12,12a,12b,13-dodecahydro-9-hydroxy-5,8a,14,14-tetramethyl-2,8-dioxo-6,13a-methano-13aH-oxeto[2″,3″:5′,6′]benzo[1′,2′:4,5]cyclodeca[1,2-d]-1,3-dioxol-4-yl ester,
d) (2R,3S)-β-(benzoylamino)-α-hydroxy benzenepropanoic acid (2aR,4S,4aS,6R,9S, 11S,12S,12aR,12bS)-6-(acetyloxy)-12-(benzoyloxy)-2a,3,4,4a,5,6,9,10,11,12,12a,12b-dodecahydro-4,11-dihydroxy-12b-[(methoxycarbonyl)oxy]-4a,8,13,13-tetramethyl-5-oxo-7,11-methano-1H-cyclodeca[3,4]benz[1,2-b]oxet-9-yl ester, and
e) (2R,3S)-β-(benzoylamino)-α-hydroxy benzenepropanoic acid (2aR,4S,4aS,6R,9S,11S,12S,12aR,12bS)-6,12b-bis(acetyloxy)-12-(benzoyloxy)-2a,3,4,4a,5,6,9,10,11,12,12a,12b-dodecahydro-11-hydroxy-4a,8,13,13-tetramethyl-4-[(methylthio)metboxy]-5-oxo-7,11-methano-1H-cyclodeca[3,4]benz[1,2-b]oxet-9-yl ester.

12. The compound of claim 10, wherein the active anti-cancer substance (Q-Y—H) is a camptothecin selected from the group consisting of
a) 4(S)-ethyl-4-hydroxy-1H-pyrano[3′,4′:6,7]indolizino[1,2-b]quinoline-3,14(4H,12H)-dione,
b) (4S)-10-[(dimethylamino)methyl]-4-ethyl-4,9-dihydroxy-1H-pyrano[3′,4′:6,7]indolizino[1,2-b]quinoline-3,14(4H,12H)-dione mono hydrochloride
c) (1S,9S)-1-amino-9-ethyl-5-fluoro-9-hydroxy-4-methyl-2,3,9,10,13,15-hexahydro-1H,12H-benzo[de]pyrano[3′,4′:6,7]indolizino[1,2-b]quinoline-10,13-dione,
d) 5(R)-ethyl-9,10-difluoro-1,4,5,13-tetrahydro-5-hydroxy-3H,15H-oxepino[3′,4′:6,7]-indolizino[1,2-b]quinoline-3,15-dione,
e) (S)-10-amino-4-ethyl-4-hydroxy-1H-pyrano[3′,4′:6,7]indolizino[1,2-b]quinoline-3,14(4H,12H)-dione,
f) 4(S)-ethyl-4-hydroxy-10-nitro-1H-pyrano[3′,4′,:6,7]-indolizino[1,2-b]quinoline-3,14(4H,12H)-dione,
g) (9S)-9-ethyl-9-hydroxy-1-pentyl-1H,1 2H-pyrano[3″,4″:6′,7′]indolizino-[1′,2′:6,5]pyrido[4,3,2-de]quinazoline-10,13(9H, 15H)-dione,
h) (9S)-9-ethyl-9-hydroxy-2-methyl-1-pentyl-1H, 12Hpyrano[3″,4″:6′,7′]indolizino-[1′,2′:6,5]pyrido[4,3,2-de]quinazoline-10,13(9H, 15H)-dione, and
i) (9S)-9-ethyl-9-hydroxy-2-hydroxymethyl-1-pentyl-1H,12H-pyrano[3″,4″:6′7′]-indolizino[1′2′:6,5]pyrido[4,3,2-de]quinazoline-10,13(9H,15H)-dione.

13. The compound of claim 10, wherein the active anti-cancer substance (Q-Y—H) is an anti-cancer nucleoside selected from the group consisting of
a) 2′-deoxy-2′,2′-difluorocytidine,
b) 2′-deoxy-2′-methylidenecytidine,
c) (E)-2′-deoxy-2′-(fluoromethylene)cytidine,
d) 1-(β-D-arabinofuranosyl)cytosine,
e) 4-amino-1-(2-deoxy-β-D-erythro-pentofuranosyl)-1,3,5-triazin-2(1H)-one,
f) 4-amino-1-[(2S,4S)-2-(hydroxymethyl)-1,3-dioxolan-4-yl]-2(1H)-pyrimidinone,
g) 2-fluoro-9-(5-O-phosphono-β-D-arabinofuranosyl)-9H-purin-6-amine, and
h) 2-chloro-2′-deoxyadenosine.

14. The compound of claim 10, wherein the active anti-cancer substance Q-Y—H is a dolastatin selected from the group consisting of
a) N,N-dimethyl-L-valyl-N-[(1S,2R)-2-methoxy-4-[(2S)-2-[(1R,2R)-1-methoxy-2-methyl-3-oxo-3-[[(1S)-2-phenyl-1-(2-thiazolyl)ethyl]amino]propyl]-1-pyrrolidinyl]-1-[(1S)-1-methylpropyl]-4-oxobutyl]-N-methyl-L-valinamide,
b) cyclo[N-methylalanyl-(2E,4E,10E)-15-hydroxy-7-methoxy-2-methyl-2,4,10-hexadecatrienoyl-L-valyl-N-methyl-kphenylalanyl-N-methyl-kvalyl-N-methyl-L-valyl-L-prolyl-N2-methylasparaginyl],
c) (1S)-1-[[(2S)-2,5-dihydro-3-methoxy-5-oxo-2-(phenylmethyl)-1H-pyrrol-1-yl]carbonyl]-2-methylpropyl ester N,N-dimethyl-L-valyl-L-valyl-N-methyl-L-valyl-L-prolyl-L-proline,
d) N,N-dimethyl-L-valyi-N-[(1S,2R)-2-methoxy-4-[(2S)-2-[(1R,2R)-1-methoxy-2-methyl-3-oxo-3-[(2-phenylethyl)amino]propyl]-1-pyrrolidinyl]-1-[(1S)-1-methylpropyl]-4-oxobutyl]-N-methyl-L-valinamide, and
e) N,N-dimethyl-L-valyl-L-valyl-N-methyl-L-valyl-L-prolyl-N-(phenylmethyl)-L-prolinamide.

15. The compound of claim 10, wherein the active anti-cancer substance (Q-Y—H) is an anthracycline selected from the group consisting of
a) (8S,10S)-10-[(3-amino-2,3,6-trideoxy-L-lyxo-hexopyranosyl)oxy]-7,8,9,10-tetrahydro-6,8,11-trihydroxy-8-(hydroxyacetyl)-1-methoxy-naphthacene-5,12-dione hydrochloride,
b) 8-acetyl-10-[(3-amino-2,3,6-trideoxy-L-lyxo-hexopyranosyl)oxy]-7,8,9,10-tetrahydro-6,8,11-trihydroxy-1-methoxy-naphthacene-5,12-dione, hydrochloride, and
c) (7S,9S)-9-acetyl-7-[(3-amino-2,3,6-trideoxy-L-lyxo-hexopyranosyl)oxy]-7,8,9,10-tetrahydro-6,9,11-trihydroxy-naphthacene-5,12-dione.

16. The compound of claim 10, wherein the active anti-cancer substance (Q-Y—H) is an EGF receptor tyrosine kinase inhibitor or a farnesyltransferase inhibitor.

17. The compound of claim 16, wherein the active anti-cancer substance (Q-Y—H) is an EGF receptor tyrosinkinase inhibitor selected from the group consisting of
a) N-(3-chloro-4-fluorophenyl)-7-methoxy-6-[3-(4-morpholinyl)propoxy]-4-quinazolinamine,
b) N-(3-ethynylphenyl)-6,7-bis(2-methoxyethoxy)-4-quinazolinamine,
c) N⁴-(3-bromophenyl)-N6-methylpyrido[3,4-d]pyrimidine-4,6-diamine, and
d) N-(3-chloro-4-((3-fluorobenzyl)oxy)phenyl)-6-(5-(((2-methylsulfonyl)ethyl)amino)-methyl)-2-furyl)-4-quinazolinamine.

18. The compound of claim 16, wherein the active anti-cancer substance (Q-Y—H) is 6-[1-amino-1-(4-chlorophenyl)-1-(1-methylimidazol-5-yl)methyl]-4-(3-chlorophenyl)-1-methylquinolin-2(1H)-one.

19. The compound of claim 8 represented by the formula (II), 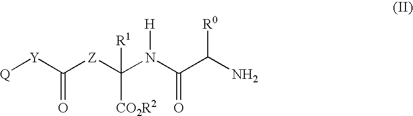
wherein
Q-Y is a radical derived from the active anti-cancer substance (Q-Y—N) in which Y is —O—, —S— or —N—,
R⁰ is a side chain of natural or non-natural amino acid,
Z is (C1-C3)alkylene or —O—CH(R³)— wherein R³ is hydrogen or straight (C1-C4)alkyl,
R¹ is hydrogen or methyl, and
R² is hydrogen, branched (C3-C10)alkyl or (C3-C8)cycloalkyl, or
a pharmaceutically acceptable salt thereof.

20. The compound of claim 19, wherein (Q-Y—H) is taxol or taxotere represented by the formula (III), 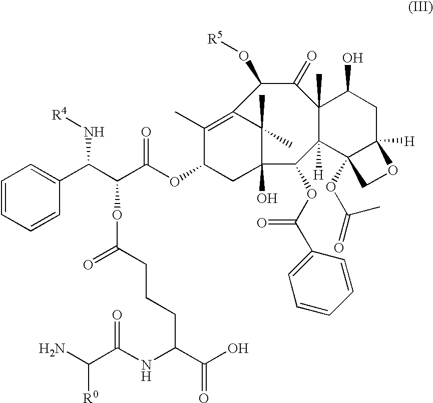
wherein
R⁰ is a side chain of natural or non-natural amino acid,
R⁴ is benzoyl or tert-butoxycarbonyl, and
R⁵ is hydrogen or acetyl,
or pharmaceutically acceptable salts thereof.

21. The compound of claim 20, wherein R⁰ is methyl, benzyl or 2-methylpropyl.

22. The compound of claim 20, selected from the group consisting of
a) 13-((2R,3S)-2-{(5S)-[5-((2S)-2-amino-4-methyl-pentanoylamino)-5-hydroxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-11-en-9-one,
b) 13α-((2R,3S)-2-{(5S)-[5-((2S)-2-amino-propinoylamino)-5-hydroxycarbonyl]pentanoyloxy}-3-benzoylamino-3-phenylpropionyloxy)-2α-benzyloxy-4α, 10β-diacetoxy-1β,7β-dihydroxy-5β,20-epoxy-tax-11-en-9-one, and
c) 13-((2R,3S)-2-{(5S)-[5-((2S)-2-amino-3-phenyl-propinoylamino)-5-hydroxycarbonyl]pentanoyloxy}-3-benzoylamino-3-phenylpropionyloxy)-2α-benzyloxy-4α,10β-diacetoxy-1β,7β-dihydroxy-5β,20-epoxy-tax-1 1-en-9-one, or
a pharmaceutically acceptable salt thereof.

23. The compound of claim 19, wherein (Q-Y—H) is an anticancer nucleoside, represented by the formula (IV), 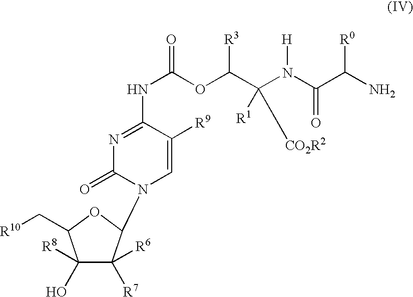
wherein
R⁰ is a side chain of natural or non-natural amino acid,
R³ is hydrogen or straight (C1-C4)alkyl,
R¹ is hydrogen or methyl, and
R² is hydrogen, branched (C3-C10)alkyl or (C3-C8)cycloalkyl, or
R⁶ is hydrogen, fluorine, hydroxyl or cyano,
R⁷ is hydrogen, fluorine or hydroxy,
or R⁶ and R⁷ taken together form methylidene or fluoromethylidene,
R⁸ is hydrogen or ethynyl,
R⁹ is hydrogen, fluorine, vinyl or ethynyl, and
R¹⁰ is hydrogen or hydroxy;
or a pharmaceutically acceptable salt thereof.

24. The compound of claim 23, wherein
R⁶ is a hydrogen, fluorine, hydroxyl,
R⁷ is a fluorine or hydroxy
or R⁶and R⁷ taken together form methylidene or fluoromethylidene.

25. The compound of claim 23, wherein
R⁰ is 2-methylpropyl, cyclohexylmethyl, 2-naphtylmethyl, 4-phenylbenzyl, (4-cyclohexylcyclohexyl)methyl, alkylthiomethyl, cyclohexylthiomethyl or 4-alkoxybenzyl, and
R³ is hydrogen or methyl.

26. The compound of claim 23 selected from the group consisting of
a) (2R)-((2S)-amino-3-cyclohexyl-propionylamino)-(3S)-[1-((4S)-hydroxy-(5R)-hydroxymethyl-3-methylene-tetrahydro-furan-(2R)-yl)-2-oxo-1,2-dihydro-pyrimidine-4-ylcarbamoyloxy]-butyric acid,
b) (2R)-((2S)-Amino-4-methyl-pentanoylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid,
c) (2R)-((2S)-Amino-3-biphenyl-4-yl-propionylamino)-(3S)-[1-((4S)-hydroxy-(5R)-hydroxymethyl-3-methylene-tetrahydro-furan-(2R)-yl)-2-oxo-1 ,2-dihydro-pyrimidin-4-ylcarbainoyloxy]-butyric acid,
d) 2(R)-[2(S)-Amino-3-biphenyl-4-yl-propionylamino)-3-{1-[4(S)-hydroxy-5(R)-hydroxymethyl-3-methylene-tetrahydro-furan-2(R)-yl]-2-oxo-1,2-dihydro-pyrimidin-4-ylcarbamoyloxy}-propionic acid,
e) (2R)-((2S)-Amino-3-naphthalen-2-yl-propionylamino)-(3S)-[1-((4S)-hydroxy-(5R)-hydroxymethyl-3-methylene-tetrahydro-furan-(2R)-yl)-2-oxo-1,2-dihydro-pyrimidin-4-ylcarbamoyloxy]-butyric acid,
f) (2R)-{(2S)-Amino-3-[4-(4-hydroxy-pbenoxy)-pbenyl]-propionylaminol-3-[1-((4S)-hydroxy-(5R)-hydroxymethyl-3-methylene-tetrahydro-furan-2-yl)-2-oxo-1,2-dihydro-pyrimidin-4-ylcarbamoyloxy]-butyric acid,
g) (2R)-[(2S)-amino-3-(4-methoxy-phenyl)-propionylamino]-(3S)-[1-[(4S)-hydroxy-(5R)-hydroxymethyl-3-methylene-tetrahydro-furan-2-yl]-2-oxo-1,2-dihydro-pyrimidin-4-ylcarbamoyloxy]-butyric acid,
h) (2R)-[(2S)-Amino-4-ethylsulfanyl-butyrylamino]-(3S)-[1-[(4S)-hydroxy-(5R)-hydroxymethyl-3-methylene-tetrahydro-furan-(2R)-yl]-2-oxo-1,2-dihydro-pyrimidin-4-ylcarbamoyl]-butyric acid,
i) (2R)-((2S)-Amino-3-cyclohexyl-propionylamino)-(3S)-[1-(3,3-difluoro-(4R)-hydroxy-(5R)-hydroxymethyl-tetrahydro-furan-2-yl)-2-oxo-1,2-dihydro-pyrimidin-4-ylcarbamoyloxy]-butyric acid,
j) 2(S)-[2(S)-amino-3-cyclohexyl-propionylamino)-3-[1-(3,3-difluoro-4(R)-hydroxy-5(R)-hydroxymethyl-tetrahydro-furan-2(R)-yl)-2-oxo-1,2-dihydro-pyrimidin-4-ylcarbamoyloxy]-2(S)-methyl-propionic acid,
k) 2(R)-[2(S)-amino-3-cyclohexyl-propionylamino]-3-{1-[3,3-difluoro-4(R)-hydroxy-5(R)-hydroxymethyl-tetrahydro-furan-2(R)-yl]-2-oxo-1,2-dihydro-pyrimidin-4-ylcarbamoyloxy}-2(R)-methyl-propionic acid,
l ) (2S,3S)-2-(2-amino-3-cyclohexyl-propionylamino)-3-[1-{(4R,5R)-3,3-difluoro-4-hydroxy-5-hydroxylmethyl-tetrahydro-furan-2-yl}-2-oxo-1,2-dihydro-pyridine-4-ylcarbamoyloxy]-2-methyl-butyric acid,
m) (2R,3R)-2-(2-amino-3-cyclohexyl-propionylamino)-3-[1-{(4R,5R)-3,3-difluoro-4-hydroxy-5-hydroxylmethyl-tetrahydro-furan-2-yl}-2-oxo-1,2-dihydro-pyridine-4-ylcarbamoyloxy]-2-methyl-butyric acid, and
n) (2R)-[(2S)-amino-3-cyclohexyl-propionylamino]-(3S)-[1-[(4S)-hydroxy-(5R)-hydroxymethyl-3-methylene-tetrahydro-furan-(2R)-yl]-2-oxo-1,2-dihydro-pyrimidine-4-ylcarbamoyloxy]-butyric acid isopropyl ester, or
a pharmaceutically acceptable salt thereof.

27. The compound of claim 19, wherein (Q-Y—H) is an anticancer nucleoside, represented by the formula (V), 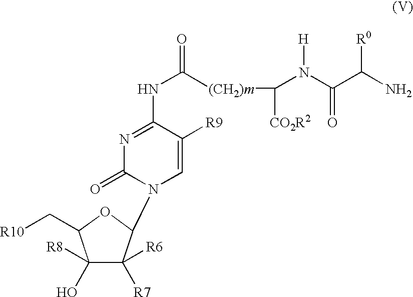
wherein
m is an integer of 2 or 3, and
R⁰, R^{2,} R⁶, R⁷, R⁸, R⁹ and R¹⁰ are as defined in claim 25, or
a pharmaceutically acceptable salt thereof.

28. The compound of claim 27, wherein
R⁶ is hydrogen, fluorine or hydroxyl,
R⁷ is fluorine or hydroxy,
or R⁶and R⁷ taken together to form methylidene or fluoromethylidene.

29. The compound of claim 27, wherein R⁰ is cyclohexylmethyl, 2-naphtylmethyl, 4-phenylbenzyl, benzyl, indol-3-ylmethyl or 4-alkoxybenzyl.

30. The compound of claim 27 selected from the group consisting of
a) (2R)-[(2S)-amino-3-(1H-indol-3-yl)propionylamino]-4-[1-((4S)-hydroxy-(5R)-hydroxymethyl-3-methylenetetrahydrofuran-2-yl)-2-oxo-1,2-dihydropyrimidin-4-ylcarbamoyl]-butyric acid,
b) (2R)-((2S)-amino-3-cyclohexylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid,
c) (2R)-((2S)-amino-3-biphenyl-4-ylpropionylamino)-4-[1-((4S)-hydroxy-(5R)-hydroxymethyl-3-methylenetetrahydrofuran-2-yl)-2-oxo-1,2-dihydropyrimidin-4-ylcarbamoyl]butyric acid, and
d) (2R)-((2S)-amino-3-naphthalen-2-ylpropionylamino)-4-[1-((4S)-hydroxy-(5R)-hydroxymethyl-3-rmiethylenetetrahydrofuran-2-yl)-2-oxo-1,2-dihydropyrimidin-4-ylcarbamoyl]butyric acid, or
a pharmaceutically acceptable salt thereof

31. The compound of claim 19, wherein (Q-Y—H) is camptothecin or its derivative, represented by the formula (VI), 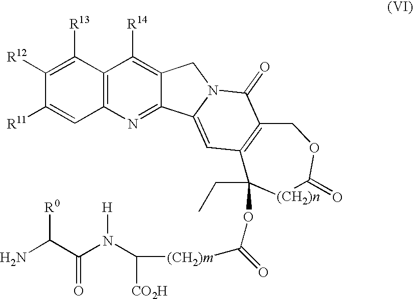
wherein
m is an integer of 1 to 3,
n is an integer of 0 to 1,
R⁰ is a side chain of natural or non-natural amino acid,
R¹¹ is hydrogen or fluorine,
R¹² is hydrogen, fluorine, methyl or hydroxy,
R¹³ is hydrogen, amino, nitro or (dimethylamino)methyl,
R¹⁴ is hydrogen, (C1-C4)alkyl, (4-methylpiperazinyl)methyl or (tert-butoxyimino)methyl or R¹³ and R¹⁴, or R¹¹ and R¹² taken together form 5 or 6 membered ring which optionally contains 1 or 2 hetero atom(s), and are optionally substituted with 1 to 3 substituant(s) selected from a group consisting of (C1-C8)alkyl, amino, (C1-C8)alkylamino and di-(C1-C4) alkylamino, or
a pharmaceutically acceptable salt thereof.

32. The compound of claim 31, wherein R¹¹ is hydrogen, R¹² is hydrogen or hydroxy, R¹³ is hydrogen or (dimethylamino)methyl and R¹⁴ is hydrogen or ethyl.

33. The compound of claim 31, wherein R⁰ is 2-methylpropyl, cyclohexylmethyl, benzyl, indol-3-ylmethyl, 4-aminobutyl, or 4-aminopropyl.

34. The compound of claim 31 selected from the group consisting of
a) 20-O—[(S)-tryptophyl-γ-(S)-glutamyl]-20-(S)-camptothecin,
b) 20-O—[(S)-valyl-γ-(S)-glutamyl]-20(S)-camptothecin,
c) 20-O—[(S)-phenylalanyl-γ-(S)-glutamyl]-20(S)-camptothecin,
d) 20-O—[(S)-leucyl-γ-(S)-glutamyl]-20(S)-camptothecin,
e) 20-O—[(R)-leucyl-γ-(S)-glutamyl]-20(S)-camptothecin,
f) 20-O—[(R)-phenylalanyl-γ-(S)-glutamyl]-20(S)-camptothecin,
g) 20-O—[(S)-tryptophyl-γ-(R)-glutamyl]-20(5)-camptothecin,
h) 20-O—[(R)-tryptophyl-γ-(R)-glutamyl]-20(S)-camptotbecin,
i) 20-O—[(S)-phenylalanyl-γ-(R)-glutamyl]-20(S)-camptothecin, and
j) 20-O—[(S)-leucyl-γ-(R)-glutamyl]-20(S)-camptothecin,
a salt free compound or a pharmaceutically acceptable salt thereof.

35. The compound of claim 31 selected from the group consisting of
a) 20-O—[(R)-tryptophyl-γ-(S)-glutamyl]-20(S)-camptothecin,
b) 20-O—[(R)-phenylalanyl-γ-(R)-glutamyl]-20(S)-camptothecin,
c) 20-O—[(R)-leucyl-γ-(R)-glutamyl]-20(S)-camptothecin,
d) 7-ethyl-10-hydroxy-20-O—[(R)-tryptophyl-(R)-homoglutamyl]-20(S)-camptothecin,
e) 7-ethyl-10-hydroxy-20-O—[(R)-tryptophyl-γ-(R)-glutamyl]-20(S)-camptothecin,
f) 7-ethyl-10-hydroxy-20-O—[(S)-phenylalanyl-γ-(R)-glutamyl]-20(S)-camptothecin,
g) 7-ethyl-10-hydroxy-20-O—[(S)-phenylalanyl-γ-(S)-aspartyl]-20(S)-camptothecin,
h) 7-ethyl-10-hydroxy-20-O—[(S)-leucyl-γ-(S)-aspartyl]-20(S)-camptothecin,
i) 20-O—[(S)-tryptophyl-β-(R)-aspartyl]-20(S)-camptothecin, and
j) 20-O—[(S)-phenylalanyl-β-(R)-aspartyl]-20(S)-camptothecin,
a salt free compound or a pharmaceutically acceptable salt thereof.

36. The compound of claim 31 selected from the group consisting of
a) 20-O—[(R)-phenylalanyl-β-(R)-aspartyl]-20(S)-camptothecin,
b) 20-O—[(S)-phenylalanyl-β-(S)-aspartyl]-20(S)-camptothecin,
c) 20-O—[(S)-leucyl-β-(R)-aspartyl]-20(S)-camptothecin,
d) 20-O—[(S)-valyl-β-(R)-aspartyl]-20(S)-camptothecin,
e) 7-ethyl-10-hydroxy-20-O—[(S)-cyclohexylalanyl-(R)-glutamyl]-20(S)-camptothecin,
f) 7-ethyl-10-hydroxy-20-O—[(S)-cyclohexylalanyl-(S)-glutamyl]-20(S)-camptothecin,
g) 20-O—[(S)-lysyl-γ-(S)-glutamyl]-20-(S)-camptothecin, and
h) 20-O—[(S)-ornithyl-γ-(S)-glutamyl]-20-(S)-camptothecin, and
i) (9S)-9-ethyl-9-[(L)-tryptophyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
a salt free compound or a pharmaceutically acceptable salt thereof.

37. The compound of claim 31 selected from the group consisting of
a) (9S)-9-ethyl-9-[(L)-cyclohexylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
b) (9S)-9-ethyl-9-[(L)-phenylalanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H, 15H)-dione hydrochloride,
c) (9S)-9-ethyl-9-[(L)-leucyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
d) (9S)-9-ethyl-9-[(L)-lysyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,
e) (9S)-9-ethyl-9-[(L)-valyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride
f) (9S)-9-ethyl-9-[(L)-ornithyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,
g) (9S)-9-ethyl-9-[(L)-leucyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione methanesulfonic acid salt,
h) (9S)-9-ethyl-9-[(D)-lysyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,
i) (9S)-9-ethyl-9-[(L)-phenylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride, and
j) (9S)-9-ethyl-9-[(L)-cyclohexylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
a salt free compound or a pharmaceutically acceptable salt thereof.

38. The compound of claim 31 selected from the group consisting of
a) (9S)-9-ethyl-9-[(L)-cyclohexylalanyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
b) (9S)-9-ethyl-9-[(L)-tryptophyl-(L)-β-aspartyloxy]-1-pentyl-1H, 12H-pyrano[3″,4″:6′,7′]indolizino[1′,2′:6,5]pyrido[4,3,2-de]quinazoline-10, 13(9H,15H)-dione hydrochloride,
c) (9S)-9-ethyl-9-[(L)-ornithyl-(D)-β-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,
d) (9S)-9-ethyl-9-[(L)-leucyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
e) (9S)-9-ethyl-9-[(L)-valyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
f) (9S)-9-ethyl-9-[(L)-leucyl-(L)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
g) (9S)-9-ethyl-9-[(L)-cyclohexylglycyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
h) (9S)-9-ethyl-9-[(D)-cyclohexylalanyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride, and
i) (9S)-9-ethyl-9-[(L)-lysyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride,
a salt free compound or a pharmaceutically acceptable salt thereof.

39. The compound of claim 31 selected from the group consisting of
a) (9S)-9-ethyl-9-[(L)-tryptophyl-(D)-γ-glutamyloxy]-1-pentyl-1H, 12H-pyrano[3″,4″:6′,7′]indolizino[1′,2′:6,5]pyrido[4,3,2-de]quinazoline-10,13(9H,15H)-dione hydrochloride,
b) (9S)-9-ethyl-9-[(L)-leucyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
c) (9S)-9-ethyl-9-[glycyl-(D)-γ-glutamyloxy]-1-pentyl-1H, 12H-pyrano[3″,4″:6′,7′]indolizino[1′,2′:6,5]pyrido[4,3,2-de]quinazoline-10,13(9H,15H)-dione hydrochloride,
d) (9S)-9-ethyl-9-[(L)-alanyl-(D)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione hydrochlorid, and
e) (9S)-9-ethyl-9-[(L)-phenylalanyl-(D)-β-aspartyloxy]-1-pentyl-1H,12H-pyrano[3″,4″:6′,7′]indolizino[1′,2′:6,5]pyrido[4,3,2-de]quinazoline-10,13(9H,15H)-dione hydrochloride,
a salt free compound or a pharmaceutically acceptable salt thereof.

40. The compound of claim 31 which is (9S)-9-ethyl-9-[(L)-lysyl-(L)-γ-glutamyloxy]-1-pentyl-1H,12H-pyrano[3″,4″:6′,7′]indolizino[1′,2′:6,5]pyrido[4,3,2-de]quinazoline-10,13(9H,15H)-dione dihydrochloride, the salt free compound or the pharmaceutically acceptable salts thereof.

41. A process for the preparation of compounds of formula (I)
X—Y-Q
wherein
X is a moiety that generates an active anti-cancer substance (Q-Y—H) selective to tumors by an enzyme; Q-Y— is a radical derived from the active anti-cancer substance (Q-Y—H) in which Y is —O—, —S— or —N—, or a pharmaceutically acceptable salt thereof, and wherein Q-Y—H is condensed with a reactive derivative of X.

42. A pharmaceutical composition containing a compound
X—Y-Q
wherein
X is a moiety that generates an active anti-cancer substance (Q-Y—H) selective to tumors by an enzyme; Q-Y— is a radical derived from the active anti-cancer substance (Q-Y—H) in which Y is —O—, —S— or —N—, or a pharmaceutically acceptable salt thereof, and wherein Q-Y—H is condensed with a reactive derivative of X.

43. The pharmaceutical composition according to claim 42 which is suitable for oral or parenteral administration.

44. A method for treating a cell proliferative disorder comprising administering to a patient in need thereof a therapeutically effective amount of an anti-cancer compound
X—Y-Q
wherein
X is a moiety that generates an active anti-cancer substance (Q-Y—H) selective to tumors by an enzyme; Q-Y— is a radical derived from the active anti-cancer substance (Q-Y—H) in which Y is —O—, —S— or —N—, or a pharmaceutically acceptable salt thereof, and wherein Q-Y—H is condensed with a reactive derivative of X.

45. The method according to claim 44 wherein the cell proliferative disorder is cancer.

46. The method according to claim 45, wherein the cancer is solid tumor.

47. The method according to claim 45, wherein the cancer is colorectal cancer, lung cancer, breast cancer, stomach cancer, cervical cancer or bladder cancer.

48. A method for creating an anti-cancer compound that is selectively activated in tumor tissues or cells, said method comprises
a) comparing the level of gene or protein expression in tumor cells or tissues and normal cells or tissues;
b) determining an enzyme capable of releasing an anti-cancer compound wherein said level of gene or protein expression in the tumor cells or tissues is at least twice the level of gene or protein expression in normal cells or tissues; and
c) selecting a moiety that is selectively cleaved in the tumor cells or tissues by said enzyme releasing the anti-cancer compound.
